(19) United States
(12) Patent Application Publication
(10) Pub. No.: US 2024/0336977 A1
(43) Pub. Date: Oct. 10, 2024

(54) MICRORNA-REGULATED BIOMARKERS AND DRUG TARGETS FOR IMPROVING DIAGNOSIS AND TREATMENT OF LUNG OR BREAST CANCER

(71) Applicant: West Virginia University Board of Governors on behalf of West Virginia University, Morgantown, WV (US)

(72) Inventor: Nancy Lan Guo, Morgantown, WV (US)

(73) Assignee: West Virginia University Board of Governors on behalf of West Virginia University, Morgantown, WV (US)

(21) Appl. No.: 18/629,544

(22) Filed: Apr. 8, 2024

Related U.S. Application Data
(60) Provisional application No. 63/494,623, filed on Apr. 6, 2023.
(60) Provisional application No. 63/509,532, filed on Jun. 22, 2023.
(60) Provisional application No. 63/515,087, filed on Jul. 21, 2023.

(51) Int. Cl. C12Q 1/6886; A61K 31/166; A61K 31/4025; A61K 31/4178; A61K 31/506; A61K 31/517; A61K 31/553; A61K 31/704

(52) U.S. Cl. CPC C12Q 1/6886; A61K 31/166; A61K 31/4025; A61K 31/4178; A61K 31/506; A61K 31/517; A61K 31/553; A61K 31/704; C12Q 2600/158; C12Q 2600/178

(57) ABSTRACT

This invention identifies sets of microRNAs (miRNAs) that classified breast cancer or lung cancer tumors from normal lung tissues with a high accuracy in patients. The findings also allowed identification of methods for improving lung or breast cancer diagnosis, optimizing treatment selection, and discovering new drug options for better patient outcomes.

### CROSS REFERENCE TO RELATED APPLICATIONS

This application claims priority to United States Provisional Application Nos. 63/494,623 filed on Apr. 6, 2023, 63/509,532, filed on Jun. 22, 2023 and 63/515,087, filed on Jul. 21, 2023, the enclosures of which are hereby incorporated in their entireties.

### STATEMENT REGARDING FEDERALLY SPONSORED RESEARCH

This invention was made with government support under GM130174, R56 LM009500, P20 RR016440, and R01 LM009500 awarded by the National Institutes of Health, and 2221895, and 2234456 awarded by the National Science Foundation. The government has certain rights in the invention.

### BACKGROUND OF THE INVENTION

### Field of the Invention

The present disclosure relates to a set of miRNAs which have been identified as differentially expressed in Non-Small Cell Lung Cancer (NSCLC) patients compared to normal controls. Processes for creating an accurate classifier for discriminating lung cancer tumors from normal lung tissues using a collected patient sample are also disclosed.

Additionally, the present disclosure relates to identifying prognostic miRNAs with The Cancer Genome Atlas lung adenocarcinoma (TCGA-LUAD) and lung squamous cell carcinoma (TCGA-LUSC) patient data, analyzing the same and identifying compounds for treating NSCLC.

The present disclosure also relates to identification of biomarkers for selecting patients with a high potential for developing invasive carcinoma in the breast with normal histology, benign legions, or premalignant lesions.

The present disclosure additionally relates to a set of miRNAs which have been identified as differentially expressed in patients with breast cancer tumors versus normal breast tissues, and compounds identified as repositioning drugs for treating the same.

This invention additionally relates to development of effective treatment strategies for metastatic breast cancer, including new and repositioned drugs to prevent and treat metastatic breast cancer.

### Description of Related Art

Lung cancer is currently the leading cause of cancer-related deaths in both men and women, accounting for approximately 27% of all deaths attributed to cancer. Additionally, lung cancer is the second most commonly occurring cancer in both sexes. Non-small cell lung cancer (NSCLC) comprises 80-85% of all lung cancer cases. Survival statistics for patients with lung cancer varies depending on the stage of cancer at diagnosis, with local and distant recurrence comprising the major causes of treatment failure. Another challenge in the treatment lies in the fact that most lung cancers are only diagnosed when the disease has al-ready reached an advanced stage, as early-stage lung cancer is often asymptomatic. Early diagnosis of lung cancer would serve to improve patient survival rates and response to therapies. Research has shown that the 5-year overall survival was markedly greater for lung cancers diagnosed in the first 2 years of screening (77%) when compared to cancers that were discovered in the third to the fifth year of screening (36%). See Bishop, J. A., et al., Accurate Classification of Non-Small Cell Lung Carcinoma Using a Novel MicroRNA-Based Approach MicroRNA-Based Approach for Lung Cancer Classification. Clinical Cancer Research, 2010. 16(2): p. 610-619.

MicroRNA (miRNA) expression profiles may provide useful information concerning tumor development and thus could serve as important diagnostic biomarkers for early disease detection. Since their discovery in 1993, miRNAs have initiated a vast amount of research not only concerning their nature but also their roles in the regulation of a multitude of biological processes to which they are attributed. The specific functions and mechanisms of miRNA have been extensively reviewed [Bartel, D. P., MicroRNAs: target recognition and regulatory functions. cell, 2009. 136(2): p. 215-233, Chua, J. H., A. Armugam, and K. Jeyaseelan, MicroRNAs: biogenesis, function and applications. Current opinion in molecular therapeutics, 2009. 11(2): p. 189-199]. Briefly, miRNAs are single-stranded, non-coding RNA molecules that average 22 nucleotides in length. They are responsible for controlling gene expression by acting as post-transcriptional regulators. In their mature form, miRNAs com-bine with proteins from the Argonaut family to form the RNA-induced silencing complex (RISC). This complex then binds to the 3′ untranslated region of various target genes and prevents their expression through either translational repression or mRNA degradation. Because each miRNA can act upon several target genes, their overall effect on the abundance of various proteins is magnified. Furthermore, miRNAs can modulate multiple points in any disease pathway. Therefore, even minor alterations in miRNA expression can result in substantial effects on protein abundance and thus, biological processes and signaling pathways [Chua, J. H., A. Armugam, and K. Jeyaseelan, MicroRNAs: biogenesis, function and applications. Current opinion in molecular therapeutics, 2009. 11(2): p. 189-199, Farazi, T. A., et al., miRNAs in human cancer. The Journal of pathology, 2011. 223(2): p. 102-115]. MiRNA expression patterns and their implications in carcinogenesis have generated extensive interest in the field of cancer biology as potential diagnostic biomarkers and therapeutic targets [Kasinski, A. L. and F. J. Slack, MicroRNAs en route to the clinic: progress in validating and targeting microRNAs for cancer therapy. Nature Reviews Cancer, 2011. 11(12): p. 849-864.].

MiRNAs have garnered interest as biomarkers in cancer diagnostics and could ultimately prove to be superior to mRNAs in this application [Ferracin, M., A. Veronese, and M. Negrini, Micromarkers: miRNAs in cancer diagnosis and prognosis. Expert review of molecular diagnostics, 2010. 10(3): p. 297-308.]. Since miRNAs are more stable than mRNA, they can be successfully isolated from formalin-fixed paraffin-embedded (FFPE) samples as well as minimally non-invasive biological samples, providing an additional asset to their diagnostic utility. The fact that miRNA expression profiles are unique to each tissue and tumor type further supports the importance of their role in cancer diagnostics [Farazi, T. A., et al., miRNAs in human cancer. The Journal of pathology, 2011. 223(2): p. 102-115.]. The presence of miRNAs in a variety of bodily fluids, such as serum, plasma, saliva, and amniotic fluid [Cortez, M. A., et al., MicroRNAs in body fluids—the mix of hormones and biomarkers. Nat Rev Clin Oncol, 2011. 8(8): p. 467-77], is a particularly fascinating but little-understood aspect of miRNA biology. Diagnostic miRNA markers were identified in tissues [Bishop, J. A., et al., Accurate Classification of Non-Small Cell Lung Carcinoma Using a Novel MicroRNA-Based Approach MicroRNA-Based Approach for Lung Cancer Classification. Clinical Cancer Research, 2010. 16(2): p. 610-619, Boeri, M., et al., MicroRNA signatures in tissues and plasma predict development and prognosis of computed tomography detected lung cancer. Proceedings of the National Academy of Sciences, 2011. 108(9): p. 3713-3718.], serum [Chen, X., et al., Identification of ten serum microRNAs from a genome-wide serum microRNA expression profile as novel noninvasive biomarkers for nonsmall cell lung cancer diagnosis. Int J Cancer, 2012. 130(7): p. 1620-8], blood [Keller, A., et al., miRNAs in lung cancer-studying complex fingerprints inpatient's blood cells by microarray experiments. BMC cancer, 2009. 9(1): p. 1-10.], and sputum samples [Yu, L., et al., Early detection of lung adenocarcinoma in sputum by a panel of microRNA markers. International journal of cancer, 2010. 127(12): p. 2870-2878] from NSCLC patients. These studies show promising results for the potential application of miRNA expression profiles as useful early diagnostic tools in a variety of non-invasive biological samples.

Intracellular and exosomal miRNAs play different roles in maintaining cellular homeostasis and intercellular communication. Intracellular miRNAs attach to particular mRNA molecules and prevent their translation within cells [Turchinovich, A. and B. Burwinkel, Distinct AGO1 and AGO2 associated miRNA profiles in human cells and blood plasma. RNA Biol, 2012. 9(8): p. 1066-75.]. Intracellular miRNAs play a role in a variety of biological functions, including development, differentiation, metabolism, and diseases including cancer, cardiovascular and neurodegenerative disorders [Bartel, D. P., Metazoan MicroRNAs. Cell, 2018. 173(1): p. 20-51.]. In contrast, exosomal miRNAs are enclosed within exosomes, which are tiny vesicles that are released by cells. MiRNAs and other proteins can be transported between cells by exosomes, which are essential for intercellular communication [Valadi, H., et al., Exosome-mediated transfer of mRNAs and microRNAs is a novel mechanism of genetic exchange between cells. Nat Cell Biol, 2007. 9(6): p. 654-9.]. Exosomal miRNAs control gene expression in target cells and participate in many biological processes, including immune control, angio-genesis, and cancer metastasis [Zhang, Y., et al., Exosomes: biogenesis, biologic function and clinical potential. Cell & Bioscience, 2019. 9(1): p. 19], and can also serve as diagnostic and prognostic biomarkers for various diseases, including cancer [Wang, M., et al., Emerging Function and Clinical Values of ExosomalMicroRNAs in Cancer. Mol Ther Nucleic Acids, 2019. 16: p. 791-804], cardiovascular disease, and infectious diseases [Mendell, J. T. and E. N. Olson, MicroRNAs in stress signaling and human disease. Cell, 2012. 148(6): p. 1172-87].

The highly conserved family of tissue-specific miRNAs keeps cells in a stable state by negatively regulating gene expression in general. Since intracellular and extracellular miRNAs have a broad range of target genes and affect almost every signaling pathway, from cell cycle checkpoints to cell proliferation to apoptosis, proper regulation of miRNA expression is necessary to maintain normal physiology. Some miRNAs function as tumor suppressors and oncogenes, and their expression is dysregulated in different cancers. Although cancer treatments are currently available to slow the growth and spread of tumors, there aren't many effective diagnostic and treatment methods for different cancers. Specific miRNA profiling can distinguish molecularly diverse tumors based on their phenotypic characteristics, which can then be used to overcome diagnostic and therapeutic obstacles [Mishra, S., T. Yadav, and V. Rani, Exploring miRNA based approaches in cancer diagnostics and therapeutics. Crit Rev Oncol Hematol, 2016. 98: p. 12-23]. The available artificial intelligence/machine learning (AI/ML) tools, bioinformatics resources, and data consor-tia accelerate the discoveries of miRNA-based theranostics.

Breast cancer is the most common female cancer worldwide and accounts for 30% of new sbcscancer cases for women each year in the United States. Despite the advances in breast cancer treatment, global breast cancer-related deaths are estimated to total 684,996 in 2020. Breast Cancer—Metastatic: Statistics. Available online: https://www.cancer.net/cancer-types/breast-cancer-metastic/statistics (accessed on Jun. 9, 2023). Most fatalities from breast cancer are caused by metastatic disease. Some key unmet clinical needs for breast cancer treatment include: (1) Early detection: Since early identification enhances the likelihood of effective treatment, improved approaches for early detection of breast cancer are required, including more precise and accessible screening tools using minimally invasive liquid biopsies. (2) Precision medicine: It is essential to develop treatment strategies that are specifically tailored to the molecular features of each patient's tumor. (3) Metastatic and triple-negative breast cancer (TNBC): TNBC is the most aggressive breast cancer subtype with limited treatment options (Paik, S.; Shak, S.; Tang, G.; Kim, C.; Baker, J.; Cronin, M.; Baehner, F. L.; Walker, M. G.; Watson, D.; Park, T.; et al. A multigene assay to predict recurrence of tamoxifen-treated, node-negative breast cancer. N. Engl. J. Med 2004, 351, 2817-2826.). The 5-year survival rate of metastatic breast cancer remains low at 28%, in contrast to 86% to 99% for women with localized or regional breast cancer (van de Vijver, M. J.; He, Y. D.; van 't Veer, L. J.; Dai, H.; Hart, A. A.; Voskuil, D. W.; Schreiber, G. J.; Peterse, J. L.; Roberts, C.; Marton, M. J.; et al. A gene-expression signature as a predictor of survival in breast cancer. N. Engl. J. Med 2002, 347, 1999-2009.). Refractory patients with failed prior therapies are generally put in hospice care. There are currently no effective diagnostic clinical tests for breast cancer using minimally invasive liquid biopsies.

Multiple breast cancer prognostic gene signatures and molecular subtypes have been applied in clinics; however, it is necessary to enhance treatment outcomes and reduce unnecessary procedures by identifying biomarkers and creating targeted therapies. miRNA based drugs need to overcome several technical difficulties, including the selection of appropriate delivery routes, management of in-vivo stability, targeting of specific tissues and cell types, and achievement of the desired intracellular effects. Due to these practical challenges, there are currently no miRNA drugs for treating breast cancer.

### SUMMARY OF THE INVENTION

This invention identifies a set of 73 microRNAs (miRNAs) that can accurately detect lung cancer tumors from normal lung tissues. Based on the consistent expression patterns associated with patient survival outcomes and in tumors vs. normal lung tissues, 10 miRNAs were considered to be putatively tumor suppressive and 4 miRNAs were deemed as oncogenic in lung cancer. From the list of genes that were targeted by the 73 diagnostic miRNAs, DGKE and WDR47 had significant associations with responses to both systemic therapies and radiotherapy in lung cancer. Based on our identified miRNA-regulated network, it was discovered three drugs BX-912, daunorubicin, and midostaurin that can be repositioned to treat lung cancer, which was not known before.

In this disclosure, relevant miRNAs were identified that are differentially expressed in NSCLC patients compared to normal controls and construct an accurate classifier for discriminating lung cancer tumors from normal lung tissues using the patient samples that were collected (n=109). By employing bioinformatics tools and statistical analyses and leveraging public data, this disclosure aims to identify a select group of miRNA biomarkers with potential clinical utility in the diagnosis of lung cancer (n=462). From the diagnostic miRNAs, it was further identified the prognostic miRNAs with The Cancer Genome Atlas lung adenocarcinoma (TCGA-LUAD) and lung squamous cell carcinoma (TCGA-LUSC) patient data (n=1,016).

The target genes of these miRNAs were then identified with TarBase. The performance of these targeted mRNAs/proteins was investigated in terms of prognosis, proliferation, and drug sensitivity. Proliferative genes were assessed using public CRISPR-Cas9 screening data in 94 human non-small cell lung cancer (NSCLC) cell lines and RNA interference (RNAi) screening data in 92 human NSCLC cell lines in the Cancer Cell Line Encyclopedia (CCLE). Drug response was analyzed using the public data in 135 human NSCLC cell lines in CCLE. Next, new compounds for treating NSCLC were determined with Connectivity Map (CMap) based on the gene expression signatures in tumorigenesis, patient survival, and proliferation. CMap provides a valuable resource for drug discovery and repositioning efforts. It allows the identification of compounds that induce similar or opposite transcriptional profiles to a query signature, thereby providing insights into potential drug mechanisms of action and novel in-dications for existing drugs. Finally, the NSCLC responders of the new compounds were identified by analyzing the CCLE profiles of drug responses.

Additionally, this disclosure identified 86 miRNAs differentially expressed between tumors and normal breast tissue samples in molecular classification. Nine miRNAs were confirmed in an external patient cohort as potential tissue-based breast cancer diagnostic biomarker. Six miRNAs has concordant differential expression between breast cancer and normal samples in both tissue and blood, with miR-30a*, miR-224*, miR-154 downregulated and miR-155, miR-1972, miR-3172 upregulated in breast cancer. Twelve miRNAs had concordant expression patterns in our collected tumors vs normal breast tissues and TCGA-BRCA patient survival hazard ratios, with five as potential oncogenes and seven as potential tumor suppressors. Using rigorous analysis of miRNA-mediated molecular machinery in patient samples, 16 protein kinase inhibitors selected by our AI pipeline are promising targeted therapies for treating breast cancer, substantiating the effectiveness of our AI/ML methods. This disclosure includes experimental agents, including MEK inhibitors PD19830 and BRD-K1224279, pilocarpine, and tremorine, as potential new drug options for improving breast cancer survival outcomes, which were previously unknown.

This disclosure identified another set of 26-gene mRNA expression profiles that were used to identify invasive ductal carcinomas from histologically normal tissue and benign tissue and to select those with a higher potential for future cancer development (ADC) in the breast associated with atypical ductal hyperplasia (AD).

### BRIEF DESCRIPTION OF THE DRAWINGS

The patent or application file contains at least one drawing executed in color. Copies of this patent or patent application publication with color drawing(s) will be provided by the office upon request and payment of the necessary fee.

Other features and advantages of the processes and compositions disclosed herein will be apparent to those skilled in the art reading the following detailed description in conjugation with the exemplary embodiments illustrated in the drawings, wherein:

FIG. 1 shows a heat map of miRNA-based clustering of patient tissue samples in the training set (MBRCC/CHTN; FDR<0.05 in SAM, fold change 0.2 or, 0.5; unpaired or paired t-tests;

FIG. 2 shows a heat map of miRNA-based clustering of patient samples in GSE15008;

FIGS. 3A and 3B show supervised prediction of lung cancer based on expression profiles of the identified miRNA markers using nearest centroid classification. FIG. 3A shows a person's correlation coefficients of correct predictions using nearest centroid algorithm. The prediction performance is shown in the table. The trend of correlation with the normal centroid was fitted with an exponentiation function, whereas the trend of correlation with the lung cancer entroid was fitted with a polynomial function. FIG. 3B shows a person's correlation coefficient of incorrect predictions in the nearest centroid classification. The trend of the correlations was fitted with logarithmic functions;

FIGS. 4A-4C show expression patterns of the identified miRNA in different histology of lung cancer. FIG. 4A shows consistently over-expressed miRNA in different histology. FIG. 4B shows consistently under expressed miRNA in different histology. FIG. 4C shows miRNA showing different expression patterns compared by histology;

FIGS. 5A-5D shows miRNA markers with a consistent expression of tumor versus normal samples in training and validation cohorts. FIG. 5A shows consistently over-expressed miRNA in lung cancer tumors in MBRCC/CHTN and GSE15008. FIG. 5B shows Consistently under-expressed miRNA in lung cancer tumors compared with GSE15008. FIG. 5C shows a heatmap of significant genes found by regular t-tests on blood samples. FIG. 5D shows expression patterns of 7 significant miRNA markers in lung cancer blood samples, tumor tissue samples in MBRCC/CHTN, and GSE15008 compared with normal samples in each cohort. “Tissue samples” refer to the samples in the MBRCC/CHTN cohort. *Statistically significant at P<0.05 in unpaired t-tests;

FIGS. 6A-6D shows the association of mRNA/protein expression of DGKE and WDR47 with the responses to selected drugs in NSCLC cell lines, where the boxes on the left indicate resistant cell lines and the boxes on the right indicate sensitive cell lines. FIG. 6A shows DGKE was expressed significantly higher in erlo-tinib-sensitive NSCLC cell lines when categorized by ln(IC50) and ln(EC50) measurements in the PRISM data. FIG. 6B shows WDR47 was expressed significantly higher in afatinib-resistant NSCLC cell lines when categorized by ln(EC50) in the PRISM data. FIG. 6C shows WDR47 was expressed significantly higher in brigatinib-resistant NSCLC cell lines when categorized by IC50 and ln(IC50) measurements in the PRISM data. FIG. 6D shows WDR47 was expressed significantly higher in osimertinib-resistant NSCLC cell lines when categorized by ln(IC50) in the GDSC2 and PRISM data;

FIG. 7 shows the mRNA expression of DGKE and WDR47 associated with radiotherapy sensitivity in TCGA-LUAD and TCGA-LUSC stage III and IV patients who had received radiotherapy, where the boxes on the left show long survival and the boxes on the right show short survival. Long survival was defined as patients surviving longer than 58 months; short survival was defined as patients surviving shorter than 20 months. DGKE and WDR47 were both expressed significantly higher in the long-survival patient group;

FIG. 8 shows CMap selected compounds: BX-912, daunorubicin, and midostaurin with a low average drug concentration in the CCLE NSCLC cell lines, where IC₅₀ is shown by the left box and EC₅₀ is shown by the right box;

FIG. 9 shows a eat map of miRNA-based clustering of patient samples (n=52). 86 miRNA markers exhibiting significant differential expression in tissue (FDR<0.05 in SAM, fold change >2 or <0.5; unpaired or paired t-tests);

FIG. 10 shows a heatmap of matched tumors and normal tissue samples from the same four breast cancer patients, showing separation of expression profiles in tumors and normal tissues (FDR<0.05 in SAM, fold change >2 or <0.5; in paired t-tests);

FIG. 11 shows a heatmap of blood samples showing clustering analysis of six miRNAs concordantly expressed between breast cancer and normal samples in tissues and blood (FDR<0.05 in SAM, P<0.05 in unpaired t-tests). Blood samples from breast cancer patients (n=3) can distinctly separate from those from normal individuals (n=6);

FIG. 12 shows a Venn diagram of selected miRNAs (isoform-specific) across different patient cohorts. The miRNA selection in the Iorio cohort and the MBRCC/CHTN breast cancer cohort was based on the statistically significant differential expression observed between breast cancer tumors and normal breast tissue samples. In TCGA, miRNA expression association with breast cancer progression was indicated with a hazard ratio (HR). HR>1: survival hazard miRNA; HR<1: survival protective miRNA;

FIG. 13 shows MiRNA-target genes and their association with pan-sensitivity/pan-resistance to 19 NCCN-recommended breast cancer drugs. The miRNAs and their experimentally validated target genes were included in Table 7;

FIGS. 14A-14C show significantly enriched cytobands and gene families of pan-sensitive and pan-resistant genes. FIG. 14A shows the −log 10(p-value) of the significantly enriched cytobands of the pan-sensitive genes. FIG. 14B shows the −log 10(p-value) of the top 10 significantly enriched cytobands of the pan-resistant genes. FIG. 14C shows the −log 10(p-value) of the top 10 significantly enriched gene families of the pan-resistant genes;

FIGS. 15A-15B show discovery of new and repositioning drugs for treating breast cancer. FIG. 15A shows the flowchart of AI pipeline for drug discovery. FIG. 15B shows the distribution of drug concentrations (IC₅₀/EC₅₀) of the 20 selected drugs in the PRISM data. All selected compounds had average IC₅₀ and EC₅₀<7 μM in the tested human breast cancer cell lines after excluding outliers;

FIG. 16 shows Hsa-miR-204 targeted genes. Among the hsa-miR-204-3p and hsa-miR-204-5p targeted genes, those pan-sensitive/pan-resistant to 19 NCCN breast cancer drugs and sensitive to BRD-K12244279 were shown;

FIG. 17A-17D shows accuracy of the 28-gene signature in prediction of breast cancer risk in two patient cohorts. FIG. 17A shows the ROC with AUC and FIG. 17B shows detailed performance in individual patients of the 28-gene signature in classifying invasive breast carcinoma from normal breast tissues in the cohort from Chen et al. [39] (n=185). FIG. 17C the ROC with AUC and FIG. 17D detailed performance in individual patients of the 28-gene signature in classifying ADH from ADHC in the cohort from Poola et al. [40] (n=8). ROC: time-dependent receiver operating characteristics curves. AUC: area under the ROC curve. ADH: atypical ductal hyperplasia. ADHC: atypical ductal hyperplasia with future cancer development. The Y axis in FIG. 17B and FIG. 17D indicated the probability in classification;

FIGS. 18A-B show RT-PCR validation of the 28-gene signature in breast cancer tumors versus normal breast tissues (n=77). FIG. 18A shows a heatmap of 25 genes separating normal breast tissues from invasive carcinoma, sarcoma, and DCIS. FIG. 18B shows a Log₂ scale fold change of each gene in each of the tumor types DCIS (n=3), invasive carcinoma (n=60), and sarcoma (n=2) vs. normal breast tissues (n=12), respectively. * p<0.05 in two-tailed unpaired t-tests. DCIS: ductal carcinoma in situ;

FIGS. 19A-B show RT-PCR validation of the 28-gene signature in blood samples (n=48). FIG. 19A shows marker genes with significant differential expression in breast cancer patient blood samples DCIS (n=3) or invasive carcinoma (n=38) vs. normal blood samples (n=7), respectively. * p<0.05 in two-tailed unpaired t-tests. FIG. 19B shows a heatmap of the mRNA expression of marker genes in breast cancer blood samples and normal blood samples. DCIS: ductal carcinoma in situ;

FIGS. 20A-F show Kaplan-Meier analysis of 25 available genes in training (FIG. 20A) and testing (FIG. 20B) sets randomly partitioned from the TCGA-BRCA data. Patients were grouped by risk scores with a cutoff value of 7.59. Among the breast cancer subtypes in TCGA-BRCA, the low-risk patient group survived significantly longer than the high-risk group for basal-like (FIG. 20C) and luminal A (FIG. 20E) patients. The plots show patient survival outcomes after surgery. The log₂ ratios of xCell scores between the high-risk and low-risk groups of basal-like (FIG. 20D) and luminal A (FIG. 20F) patients are presented. All results presented were statistically significant. Statistical analysis was performed using two-sample t-tests to assess the difference between the two groups (*: p<0.05; **: p<0.01; ***: p<0.001);

FIGS. 21A-E show the correlation between the immune infiltration level and gene expression, estimated with TIMER, in different subtypes of breast cancer patients. FIG. 21A shows the correlation in all TCGA-BRCA patients (n=1100). FIGS. 21B-E show the correlation in specific subtypes of BRCA, including basal-like (n=191), Her2-positive (n=82), luminal A (n=568), and luminal B (n=219). ‘NA’ indicates that the data are not available;

FIGS. 22A-D show protein expression validation of the marker genes. FIG. 22A shows protein expression of PBX2, RAD50, and RAD52 in cell lines MCF10A, MCF7, AU565, BT474, and MDA453 in Western blots. FIG. 22B shows immunohistochemistry (IHC) staining of RAD52 in breast cancer tumor tissue. FIG. 22C shows IHC staining of PBX2 in breast cancer tumor tissue. Both FIGS. 22B and 22C were shot with an objective of 40×. FIG. 22D shows TIC with control, RAD52, and PBX2 in normal breast tissue, viewed at 20×;

FIGS. 23A-F show Kaplan-Meier curves of RAD52 and PBX2 protein expression in immunohistochemistry (IHC) for breast cancer overall survival and recurrence-free survival. IHC scores of RAD52 are shown in FIGS. 23A and 23B. IHC scores of PBX2 are shown in FIGS. 23C and 23D and were used in patient stratification, respectively. The combined RIC scores of RAD52 and PBX2 were used to stratify patients into different prognostic groups in overall survival shown in FIG. 23E and recurrence-free survival shown in FIG. 23F analyses;

FIGS. 24A-24F show patient survival analysis and immune cell type activities in patient tumors grouped by the expression level of RAD52 and PBX2 in TCGA-BRCA. FIG. 24A shows when RAD52 mRNA was overexpressed, patients survived for a shorter length of time. Kaplan-Meier analysis was not significant. FIG. 24C shows when PBX2 mRNA was overexpressed, patients survived significantly longer. FIG. 24E shows when the patients were grouped by the expression level of RAD52 and PBX2 together, the patient group with both genes expressed lower had the longest survival time among the four groups; the patient group with higher RAD52 mRNA expression and lower PBX2 mRNA expression had the shortest length of survival time among the four groups. FIGS. 24B and 24D show the log₂ ratio of xCell scores of patient samples grouped by the mRNA expression level of RAD52 and PBX2, respectively. Cell types that had significantly different activities between the high- and low-expression groups in two-sample t-tests were included. FIG. 24F shows the F-statistics of xCell scores grouped by the mRNA expression level of RAD52 and PBX2. Cell types that were significant in the ANOVA tests were included. *: p<0.05; **: p<0.01; ***: p<0.001.

FIG. 25 shows the proliferation of selected genes in human breast cancer (BRCA) cell lines as analyzed through CRISPR-Cas9 and RNAi screening. Fore each gene, the count of BRCA cell lines with significant dependency scores (<−0.5) is shown for CRISPR-Cas9 (n=48) and RNAi (n=34), respectively. Genes that did not have a significant impact on BRCA cell lines were not included in this figure; and,

FIG. 26 shows the average drug concentration of selected drugs in the CCLE human BRCA cell lines (n=22). Dasatinib had a low average concentration of drug response across the cell lines tested. The measurements of EC₅₀=4.49×10⁷ μM of dasatinib in cell line HCC38 and EC₅₀=24.52 μM of saracatinib in cell line HCC1937 were outliers and removed from the plots.

### DESCRIPTION OF THE INVENTION

As used herein, the term “patient” refers to any member of the animal kingdom, including, for example, but not limited to human beings.

As used herein, the term “therapeutically effective amount” refers to that amount of a compound or pharmaceutical composition to bring about a desired effect, such as treating a patient.

### Non-Small Cell Lung Cancer

### Materials and Methods

Patient Samples. A total of 117 patient tissue samples were procured from the WVU Tissue Bank (Morgantown, WV) and the Cooperative Human Tissue Network (CHTN) and stored at −80° C. These samples included 109 snap-frozen lung tumor samples, as well as 22, matched normal lung tissue samples. The samples were examined by pathologists before being cataloged in the biorepositories at WVU tissue bank and CHTN. For tumor tissue samples, it was certified that the tumor content is at least 50% in each sample. For non-cancerous normal lung tissues, it was confirmed that no tumor was present in the sample. This information was provided in the pathology reports of the tissue samples that were received. Before our experiments, a pathologist's assistant further examined the tissue samples. No experimental control was carried out to confirm that there are no tumor cells in the normal tissues. Blood samples from 4 lung cancer patients and 6 normal individuals were provided by CHTN and were sent frozen, on dry ice following collection in EDTA blood tubes. These samples were also stored at −80° C. until use. Pathology reports were provided with all samples to verify cancer stage, tumor grade, and histology. All identifying notations were removed from the pathology reports prior to delivery to ensure patient privacy was maintained. This study was approved with an Institutional Review Board exemption from West Virginia University. This cohort (MBRCC/CHTN) was used as the training set to identify diagnostic miRNA markers (Table 1).

TABLE 1
Patient clinical information, including AJCC cancer stage,
age group, gender, MBRtumor grade, and tissue histology

| Clinical Information | N |
|---|---|
| Cancer Stage | 109 |
| 1 | 40 |
| 2 | 34 |
| 3 | 13 |
| Normal | 22 |
| Age | |
| <60 | 23 |
| 60-80 | 68 |
| >80 | 2 |
| Missing | 16 |
| Gender | |
| Male | 57 |
| Female | 51 |
| Missing | 1 |
| Tumor Grade | |
| 1 | 4 |
| 2 | 29 |
| 3 | 42 |
| Normal | 22 |
| Missing | 12 |
| Histology | |
| NSCLC | |
| Adenocarcinoma | 41 |
| Adenosquamous | 1 |
| carcinoma | |
| Large cell carcinoma | 2 |
| Non-small cell carcinoma | 13 |
| Sarcomatoid carcinoma | 1 |
| Squamous cell carcinoma | 1 |
| Small Cell Carcinoma | 1 |
| Carcinoid tumor | 2 |
| Normal Tissue | 22 |

A validation cohort (n=375) was retrieved from the NCBI Gene Expression Omnibus database with accession number (GSE15008). This cohort contains 187 lung cancer tumor tissue samples and 188 normal lung tissue samples, most of which were from adjacent normal match tissues.

The Cancer Genome Atlas (TCGA) is a publicly accessible database that provides a comprehensive resource for genomic data on various types of cancer. The study obtained miRNA expression, gene expression, and mutation data for TCGA lung adenocarcinoma (TCGA-LUAD; http://linkedomics.org/data_download/TCGA-LUAD/, accessed on 10 Jan. 2023) and TCGA lung squamous cell carcinoma (TCGA-LUSC; http://linkedomics.org/data_download/TCGA-LUSC/, accessed on 10 Jan. 2023) from LinkedOmics [Vasaikar, S. V., et al., LinkedOmics: analyzing multi-omics data within and across 32 cancer types. Nucleic Acids Res, 2018. 46(D1): p. D956-D963]. Also included was a total of 63 LUAD and 136 LUSC normalized miR-gene level samples from the Illumina GenomeAnalyzer platform, as well as 450 LUAD and 342 LUSC normalized miR-gene level samples from the Illumina HiSeq platform. The RNA sequencing data for LUAD (n=515) and LUSC (n=501) was measured using the Illumina HiSeq 2000 RNA Sequencing platform. The miRNA and gene expression data were log 2 transformed. Patient clinical information was obtained from LinkedOmics.

There was also conducted a prognostic evaluation in non-small cell lung cancer (NSCLC) using RNA sequencing data from Xu et al [Xu, J. Y., et al., Integrative Proteomic Characterization of Human Lung Adenocarcinoma. Cell, 2020. 182(1): p. 245-261.e17]. Log 2-transformed mRNA data of 51 LUAD tumors and 49 paired NATs used in this study was obtained from Xu's LUAD cohort.

RNA Isolation and Quality Assessment. Total RNA was extracted from frozen tumor and normal tissue samples using the mirVana miRNA Isolation kit and following the manufacturer's protocol (Ambion, Austin, TX). Total RNA was isolated from frozen blood samples using a modified PAXgene protocol that was detailed by Beekman et al [Beekman, J. M., et al., Recovery of microarray-quality RNA from frozen EDTA blood samples. Journal of pharmacological and toxicological methods, 2009. 59(1): p. 44-49]. Briefly, frozen blood samples were thawed on ice and transferred to PAXgene blood collection tubes. Extraction of total RNA was performed using the PAXgene Blood miRNA kit according to the manufacturer's instructions (Qiagen, Valencia, CA). RNA concentration was determined using the NanoDrop 1000 Spectrophotometer (NanoDrop Technologies, Wilmington, DE), and RNA quality was assessed using the 2100 Bioanalyzer (Agilent Technologies, Santa Clara, CA). RNA quality was checked with UV Spectrometry (260/280>1.8) and RNA quality determination gel (1% agarose-2% formaldehyde QC gel). In all, total RNA extracted from 109 patient tissue samples and 10 patient blood samples met the quality control criteria and were selected for further analysis.

Microarray Analysis. MiRNA profiling, including additional quality controls, was completed by Ocean Ridge Biosciences (Palm Beach Gardens, FL) using custom microarrays containing 1,087 human miRNA probes. These miRNA arrays incorporate all 1,098 human miRNAs present in the Sanger Institute mirBASE version 15. Quality control features that were included in this analysis consist of negative controls, specificity controls, and spiking probes. Furthermore, a detection threshold was calculated for each array by summing five times the standard deviation of the background signal and the 10% trim mean of the negative control probes. Using these values, it was possible to filter out probes with uniformly low signals prior to completing our statistical analysis. The miRNA microarray data were quantile normalized between arrays. The raw microarray data and patient clinical information are available in NCBI Gene Expression Omnibus with accession number GSE31275.

Hierarchical clustering analysis and heatmap. Hierarchical two-dimensional clustering analyses were performed using the expression profiles of the identified miRNA markers, with the Heatplus function in the R package. Similarity metrics were Manhattan distance, and the cluster method was Ward's linkage. Heatmaps were then generated in the R package.

Nearest Centroid Classification. In the external validation of the lung cancer diagnostic model, the nearest centroid method was used to classify lung cancer samples from normal lung tissues. Specifically, Pearson's correlation coefficients between a new patient's miRNA expression profiles and those of the lung cancer centroid and the normal centroid in the training cohort were computed, respectively. A patient sample was predicted as lung cancer if the correlation with the lung cancer centroid was greater than that with the normal centroid; otherwise, it was predicted as normal.

MiRNA Targeted Genes. TarBase [Karagkouni, D., et al., DIANA-TarBase v8: a decade-long collection of experimentally supported miRNA-gene interactions. Nucleic Acids Res, 2018. 46(D1): p. D239-D245] is a comprehensive database that provides a centralized resource for information on experimentally validated microRNA-target interactions. The bulk download modules of TarBase v7.0 (http://diana.imis.athena-innovation.gr/DianaTools/data/TarBase7data.tar.gz, accessed on 10 Jan. 2023) and v8.0 (https://dianalab. e-ce.uth.gr/html/diana/web/index.php?r=tarbasev8%2Fdownloaddataform, accessed on 10 Jan. 2023) were obtained, experimentally confirmed target genes of selected miRNAs were retrieved.

Cancer Cell Line Encyclopedia. RNA sequencing data was obtained of 135 cell lines, 108,344 mutations, and 4,223 fu-sions for NSCLC from the Cancer Cell Line Encyclopedia (CCLE) release DepMap Public 22Q2 [DepMap, DepMap 22Q2 Public 2022: In Broad: figshare.](https://depmap.org/portal/download/all/, accessed on 25 Jan. 2023). In addi-tion, from the CCLE 2019 release [Ghandi, M., et al., Next-generation characterization of the Cancer Cell Line Encyclopedia. Nature, 2019. 569(7757): p. 503-508.] (https://depmap.org/portal/download/all/, accessed on 25 Jan. 2023), miRNA data of 123 NSCLC cell lines were collected. From the study conducted by the Gygi lab [Nusinow, D. P., et al., Quantitative Proteomics of the Cancer Cell Line Encyclopedia. Cell, 2020. 180(2): p. 387-402.e16](https://gygi.hms.harvard.edu/publications/ccle.html, accessed on 26 Jan. 2023), it was acquired proteomic data for 63 NSCLC cell lines. The mRNA and proteomic data are both log 2-tranformed. Furthermore, the mean of protein expression was centered at 0. These datasets provide a comprehensive resource for molecular profiles of NSCLC cell lines, enabling the investigation of the molecular characteristics and potential therapeutic targets of NSCLC.

Drug Response. The PRISM (Profiling Relative Inhibition Simultaneously in Mixtures) framework [Corsello, S. M., et al., Discovering the anti-cancer potential of non-oncology drugs by systematic viability profiling. Nat Cancer, 2020. 1(2): p. 235-248] is a computational tool that estimates drug sensitivities of cancer cell lines using molecular profiling data, particularly gene expression data from the CCLE database. It was obtained the PRISM repurposing dataset of the secondary screen from DepMap release PRISM Repur-posing 19Q4 (https://depmap.org/portal/download/all/, accessed on 25 Jan. 2023). For this study, the PRISM drug response data was used from 94 NSCLC cell lines for 1,448 compounds to investigate the drug sensitivity of specific genes.

The Genomics of Drug Sensitivity in Cancer (GDSC) [Yang, W., et al., Genomics of Drug Sensitivity in Cancer (GDSC): a resource for therapeutic biomarker discovery in cancer cells. Nucleic Acids Res, 2013. 41(Database issue): p. D955-61] database is a publicly available resource that provides comprehensive data on drug responses in a large panel of cancer cell lines. The drug screening data for this study were obtained from the project website (https://www.cancerrxgene.org/, accessed on 25 Jan. 2023). The drug response data from 78 NSCLC CCLE datasets was used from GDSC1 and 69 NSCLC CCLE datasets from GDSC2. Details of the method used to categorize each cell line's drug sensitivity were previously published by our lab [Ye, Q., et al., A Multi-Omics Network of a Seven-Gene Prognostic Signature for Non-Small Cell Lung Cancer. Int J Mol Sci, 2021. 23(1), Ye, Q., et al., Immune-Omics Networks of CD27, PD1, and PDL1 in Non-Small Cell Lung Cancer. Cancers (Basel), 2021. 13(17)]

Proliferation Assays. Gene knockout effects in CCLE using CRISPR-Cas9 screens [Meyers, R. M., et al., Computational correction of copy number effect improves specificity of CRISPR-Cas9 essentiality screens in cancer cells. Nat Genet, 2017. 49(12): p. 1779-1784] were quantified in Project Achilles and available from DepMap Public 22Q2 release (https://depmap.org/portal/download/all/, accessed on 25 Jan. 2023). The gene-level CRISPR-Cas9 dependency scores were standardized using the CERES method. For genome-scale screening data of RNA interference (RNAi) knockdown [McFarland, J. M., et al., Improved estimation of cancer dependencies from large-scale RNAi screens using model-based normalization and data integration. Nat Commun, 2018. 9(1): p. 4610] for NSCLC cell lines, the Project Achilles dataset was accessed via the website of the DepMap portal (https://depmap.org/R2-D2/, accessed on 25 Jan. 2023). For each cell line, the average gene dependency scores were estimated with the DEMETER2 v6 algorithm.

To determine the impact of gene knockout in NSCLC cell lines, it was analyzed genome-wide CRISPR-Cas9 knockout dependency scores for 94 NSCLC cell lines and RNAi screening data for 92 NSCLC cell lines. Genes were classified as essential or non-essential based on their importance to cell growth in each line. For the normalization of the gene ef-fects in each cell line, the median knockout effect value of the essential gene was set to −1 and that of the non-essential gene was set to 0. A normalized dependence score of less than −0.5 was considered to have a significant effect on CRISPR-Cas9 knockout and RNAi knockdown.

Connectivity Map. Connectivity Map [Subramanian, A., et al., A Next Generation Connectivity Map: L1000 Platform and the First 1,000,000 Profiles. Cell, 2017. 171(6): p. 1437-1452.e17, Lamb, J., et al., The Connectivity Map: using gene-expression signatures to connect small molecules, genes, and disease. Science, 2006. 313(5795): p. 1929-35] (CMap; https://clue.io/, accessed on 25 Jan. 2023) is a public database that contains gene expression profiles of human cells treated with various bioactive small molecules. In this study, it was identified gene expression signatures and discovered their potential connected repositioning drugs by utilizing the CMap web tool. The disclosure considered connectivity scores to be significant if they exceeded 0.9 and had a p-value of 0.05 or below.

Statistical Analysis. The Significance Analysis of Microarrays (SAM) method [Tusher, V. G., R. Tibshirani, and G. Chu, Significance analysis of microarrays applied to the ionizing radiation response. Proceedings of the National Academy of Sciences, 2001. 98(9): p. 5116-5121] was used to identify miRNA markers exhibiting differential expression in lung cancer vs. normal samples. The following cut-off values were selected to evaluate those miRNA markers that demonstrated an expression fold change of >2 or <0.5 between normal and tumor tissues or between blood samples from normal or cancer patients; P<0.05; false discovery rate (FDR)<0.05, using unpaired t-tests, or paired t-tests based on the sample set. Student's t-tests were used to compare the gene expression of the two groups, and a two-sided p-value of 0.05 or below was deemed statistically significant. This study conducted survival analysis using the Kaplan-Meier method with the survival package in R. To evaluate differences in survival probabilities across groups, it was performed log-rank tests on the Kaplan-Meier curves. All the statistical analyses were performed using Rstudio on R version 4.2.1.

### Results

Identification of diagnostic miRNA markers. Using the MBRCC/CHTN cohort (Table 1) as the training set, 66 miRNAs had significant differential expression (P<0.05, unpaired t-tests; FDR<0.05, SAM) with at least a 2-fold change (either over-expression or under-expression) in 87 lung cancer samples and 22 normal lung tissues. When 22 lung cancer tumor samples and matched normal lung tissues were analyzed, 58 miRNA showed significant differential expression (P<0.05, unpaired t-tests; FDR<0.05, SAM) with at least a 2-fold change. Among these identified miRNA markers, 51 were common in both sets, leading to a set of 73 unique miRNA diagnostic markers.

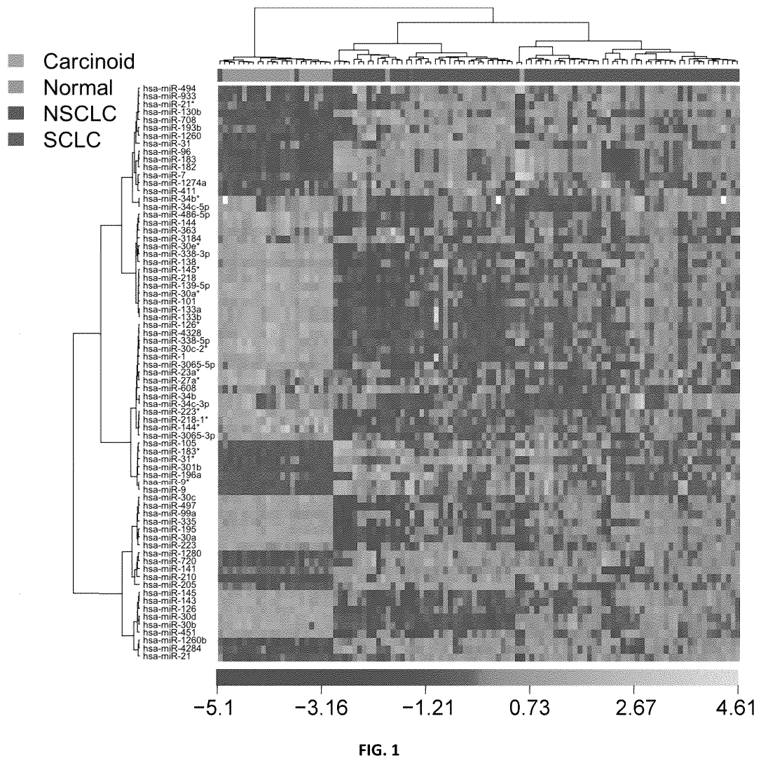

As seen in FIG. 1, using the expression profiles of these 73 miRNA markers, patient samples in the MBRCC/CHTN were separated into two groups with distinct miRNA expression patterns. Specifically, the left panel in FIG. 1 contains normal samples with 2 NSCLC and 1 carcinoid sample misclassified into this cluster; the right panel in FIG. 1 contains lung cancer samples, with 1 normal tissue misclassified into this cluster. Overall, the overall accuracy of the unsupervised hierarchical clustering was 96.3% (105/109), with a sensitivity of 96.6 (84/87) and a specificity of 95.5% (21/22).

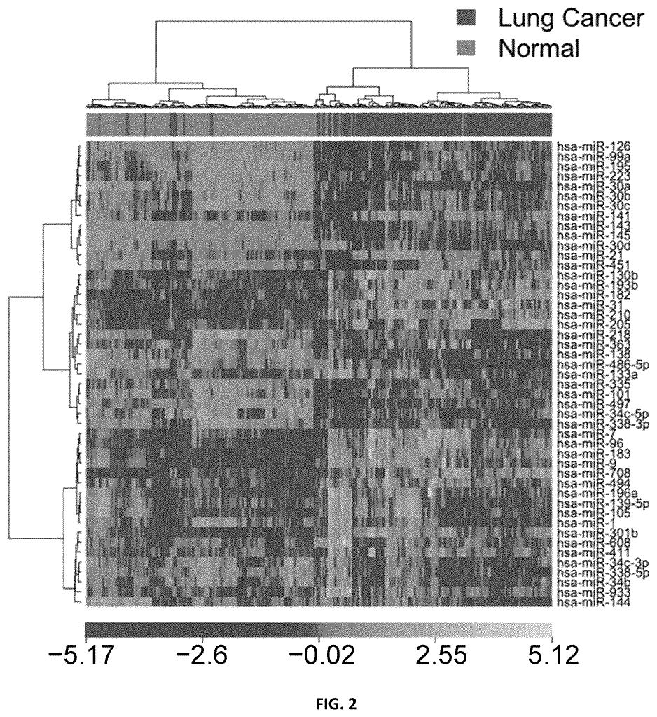

To validate the miRNA-based diagnostic model, both unsupervised and supervised methods were used to classify lung cancer samples from normal lung tissue samples in an external cohort (n=375; GSE15008). Out of 73 identified miRNA markers, 47 were found in the validation set. By using the expression profiles of these 48 miRNA markers, lung cancer samples were separated from the normal lung tissues in unsupervised hierarchical clustering with the same statistical parameters (FIG. 2). There were 174/187 (93%) correctly classified cancer samples and 170/188 (90.4%) correctly classified normal tissue samples, giving an overall accuracy of 91.7% in unsupervised classification, indicating that the identified miRNA markers could separate lung cancer and normal lung tissues with distinct expression patterns.

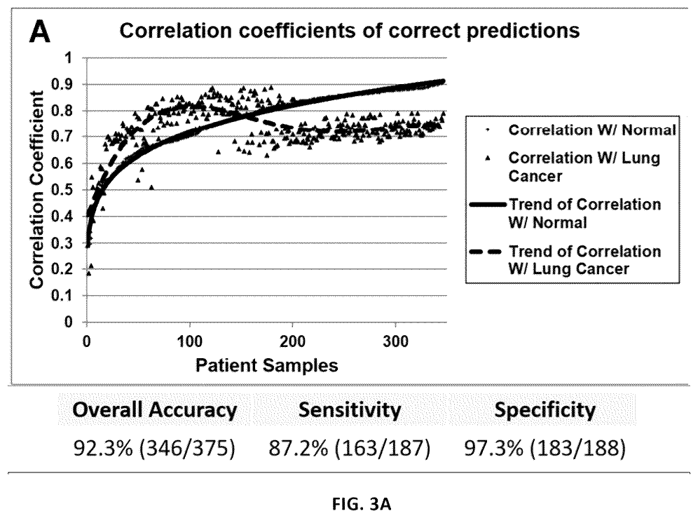

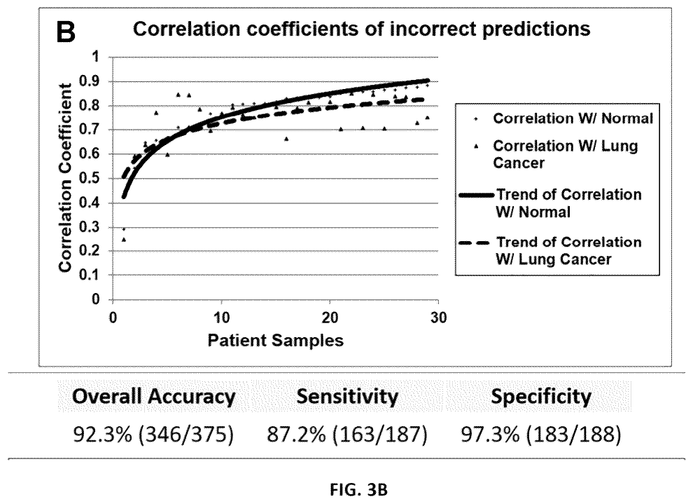

To further validate the potential clinical utility of the miRNA-based diagnostic model, the nearest centroid method was used to predict lung cancer cases in the external validation set. The “lung cancer centroid” and “normal centroid” with the 4 misclassified samples in the training cohort were used in the classification. Each sample in the GSE15008 cohort was classified based on its miRNA expression correlations with the “lung cancer centroid” and the “normal centroid” in the training set. A patient was predicted as lung cancer if the correlation with the “lung cancer centroid” was greater than that with the “normal centroid”; otherwise, a patient sample was predicted as normal. In general, when the correlation coefficient with the “normal centroid” is less than 0.8, the sample was most likely a lung cancer tumor (FIG. 3A). For most misclassified samples, the correlation coefficients with both centroids were very close and hard to distinguish (FIG. 3B). The overall accuracy of the lung cancer prediction is 92.3% (346/375), with a sensitivity of 87.2% (163/187) and a specificity of 97.3% (183/188). The miRNA profiles of the training and testing cohorts were based on different miRNA platforms. The highly accurate validation results demonstrate that the miRNA expression-based model could accurately predict lung cancer risk and could be used for diagnostic tests.

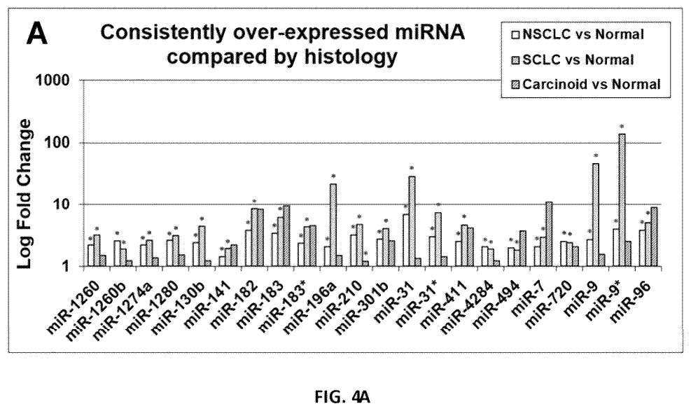

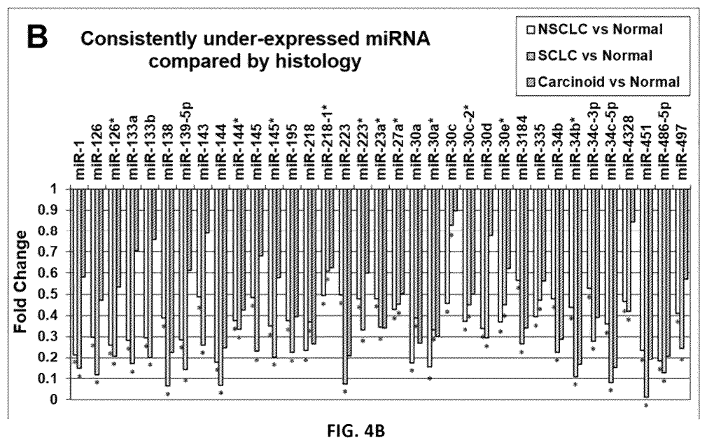

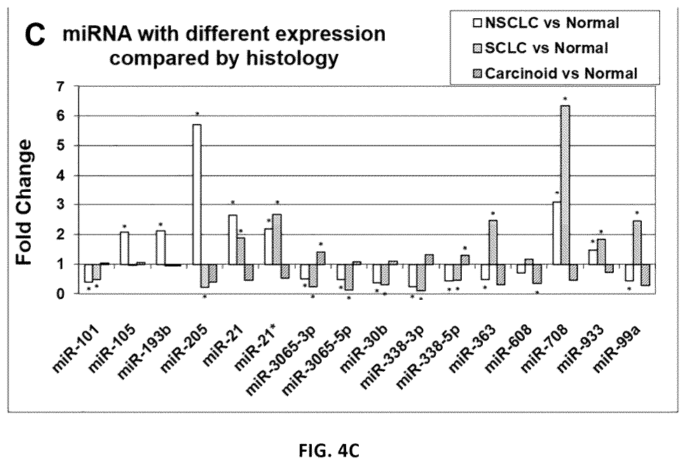

Comparison of miRNA markers in different histology. After substantiating the diagnostic capacity of the identified 73 miRNA markers for lung cancer, it was sought to investigate their expression patterns in different histology of lung cancer, including NSCLC, small cell lung cancer (SCLC), and carcinoid in the MBRCC/CHTN co-hort. A total of 22 miRNA had consistent over-expression in these three histological types (FIG. 4A). Among these 22 miRNAs, hsa-miR-210 was significantly overexpressed in SCLC, NSCLC, and carcinoid (P<0.05; unpaired t-tests). The other 21 miRNAs were significantly overexpressed (P<0.05; unpaired t-tests) in SCLC and NSCLC, but their over-expression was not significant in carcinoid. Notably, hsa-miR-9* had a more than 100-fold overexpression in SCLC (P<0.05; unpaired t-tests). A total of 35 miRNAs were under-expressed in SCLC, NSCLC, and carcinoid (FIG. 4B). All of these 35 miRNAs were significantly under-expressed in SCLC and NSCLC, but their under-expression not significant in carcinoid (P<0.05; unpaired t-tests). A total of 16 miRNAs had different expression patterns in SCLC, NSCLC, and carcinoid (FIG. 4C). Specifically, hsa-miR-3065-3p and hsa-miR-338-5p was over-expressed in carcinoid, but were under-expressed in SCLC and NSCLC (P<0.05; unpaired t-tests), indicating that these two miRNA markers could potentially be used for classification of lung cancer histological subtypes.

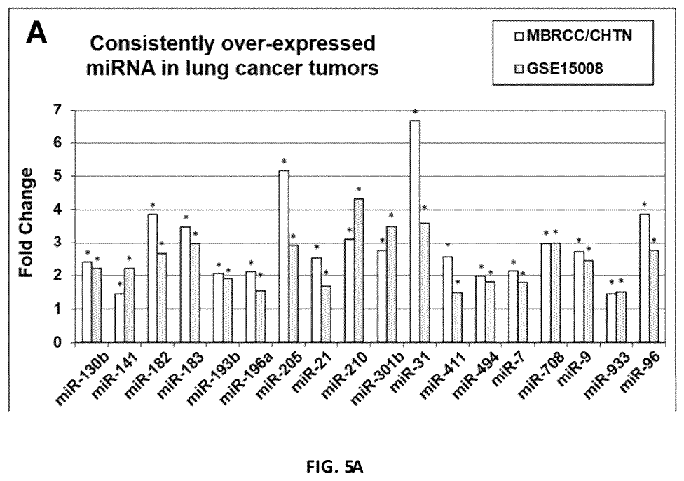

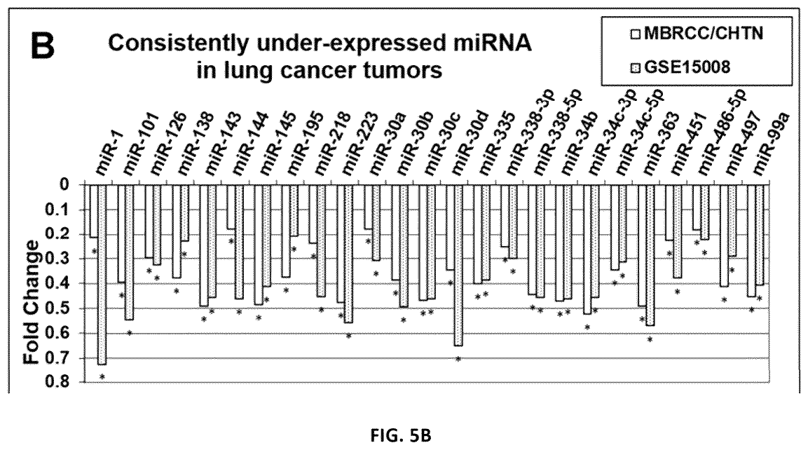

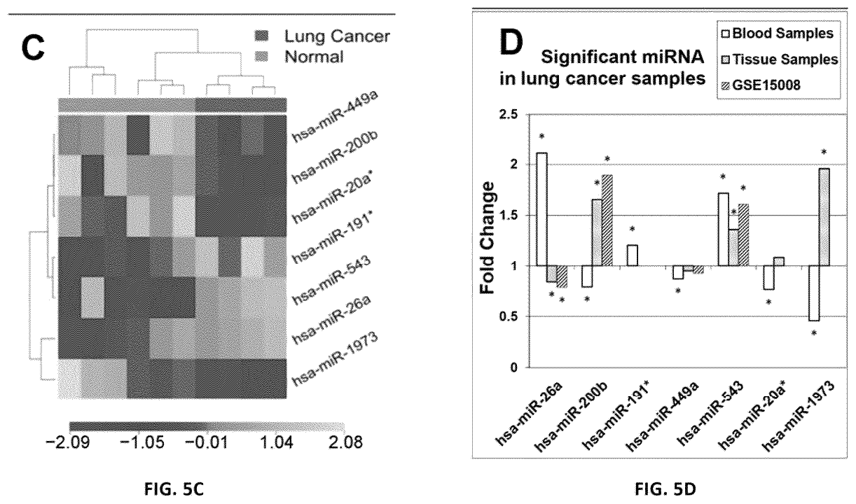

Confirmation of miRNA expression patterns in multiple cohorts. The expression patterns of the identified miRNA markers were confirmed with the external validation set. A total of 18 miRNAs had consistent over-expression (P<0.05; un-paired t-tests) in lung cancer tumor tissues (FIG. 5A), including hsa-miR-130b, hsa-miR-141, hsa-miR-182, hsa-miR-183, hsa-miR-193b, hsa-miR-196a, hsa-miR-205, hsa-miR-21, hsa-miR-210, hsa-miR-301b, hsa-miR-31, hsa-miR-411, hsa-miR-494, hsa-miR-7, hsa-miR-708, hsa-miR-9, hsa-miR-933, and hsa-miR-96.

A total of 25 miRNAs were consistently under-expressed (P<0.05; unpaired t-tests) in lung cancer tissue samples in both cohorts (FIG. 5B), including hsa-miR-1, hsa-miR-101, hsa-miR-126, hsa-miR-138, hsa-miR-143, hsa-miR-144, hsa-miR-145, hsa-miR-195, hsa-miR-218, hsa-miR-223, hsa-miR-30a, hsa-miR-30b, hsa-miR-30c, hsa-miR-30d, hsa-miR-335, hsa-miR-338-3p, hsa-miR-338-5p, hsa-miR-34b, hsa-miR-34c-3p, hsa-miR-34c-5p, hsa-miR-363, hsa-miR-451, hsa-miR-486-5p, hsa-miR-497, and hsa-miR-99a.

MicroRNA markers in blood samples. The disclosure sought to identify miRNA markers in blood samples from lung cancer patients (n=4) and normal individuals (n=6). Seven miRNAs showed significant differential expression (P<0.05; unpaired t-tests) in lung cancer versus normal samples (FIG. 5C). Specifically, hsa-miR-26a and hsa-miR543 were over-expressed in lung cancer; whereas hsa-miR-200b, hsa-miR-449a, hsa-miR-20a*, and hsa-miR-1973 were under-expressed in lung cancer (FIG. 5D). When compared with the expression patterns in the tissue samples in the two patient cohorts, hsa-miR-543 had concordant overexpression in both lung cancer tumor tissues and blood (P<0.05; unpaired t-tests). hsa-miR-26a was over-expressed in the lung cancer blood but under-expressed in the tumor tissues; whereas hsa-miR-200b was under-expressed in the blood but over-expressed in the tumor tissues in both clinical cohorts (P<0.05; un-paired t-tests).

Identification of Prognostic miRNAs To demonstrate the prognostic performance of the selected miRNAs, the survival analysis of each miRNA was performed on the TCGA-LUAD and TCGA-LUSC datasets. For each miRNA, it was defined a log 2-transformed expression cutoff to divide the patients into groups of overexpression and underexpression and selected the cutoff point that yielded the smallest P-value in the statistical survival analysis between the groups. The miRNAs were categorized with a hazard ratio>1 and a P-value<0.05 and overexpressed in lung cancer tumors (FIG. 5A) as potential oncogenic miRNAs, and those with a hazard ratio<1 and a P-value<0.05 and underexpressed in lung cancer tumors (FIG. 3B) as potential tumor suppressive miRNAs (Table 2). There were 14 miRNAs with concordant categorization results in lung cancer tumors vs. TCGA-LUAD, TCGA-LUSC, and TCGA-NSCLC (combined TCGA-LUAD and TCGA-LUSC).

TABLE 2
Potential tumor suppressive and potential oncogenic miRNAs.

| Potential tumor suppressive miRNAs | Potential oncogenic miRNAs |
|---|---|
| 4, hsa-miR-195, hsa-miR-223, hsa-miR- | hsa-miR-21, hsa-miR-31, hsa- |
| R-30b, hsa-miR-30d, hsa-miR-335, | miR-411, hsa-miR-494 |
| hsa--363, hsa-miR-451, hsa-miR-99a | |

Association between miRNA-Targeted Genes and Responses to Systemic Therapies and Radiotherapy To demonstrate the clinical relevance and support further investigation of the selected diagnostic miRNAs, it was retrieved experimentally confirmed target genes using TarBase. A total of 3,139 genes were identified as target genes of our discovered diagnostic miRNAs.

The CCLE drug screening data was utilized, which included 21 drugs recommended by the National Comprehensive Cancer Network (NCCN) for systemic or targeted therapy in the treatment of NSCLC. The disclosure aimed to identify genes that were pansensitive or panresistant to the 21 NCCN-recommended drugs in NSCLC among all the selected miRNA and their targeted genes. The disclosure defined pansensitive genes/miRNAs as those that exhibited sensitivity or lack of resistance to all 21 studied drugs, and panresistant genes/miRNAs as those that exhibited resistance or lack of sensitivity to all 21 studied drugs. The disclosure analyzed RNA sequencing, proteomics, and miRNA profiles of human NSCLC cell lines in the CCLE. Specifically, genes/miRNAs with significantly higher expression (P<0.05; two-sample t-tests) in NSCLC cell lines sensitive to a specific drug were classified as sensitive genes/miRNAs, whereas genes/miRNAs with significantly higher expression (P<0.05; two-sample t-tests) in NSCLC cell lines resistant to the same drug were classified as resistant genes/miRNAs. In this analysis, only the genes that were consistently pansensitive or panresistant at both mRNA and protein levels were selected, and the miRNAs that were pansensitive or panresistant (Table 3).

TABLE 3
Pansensitive and panresistant genes concordant in both mRNA and proteomics
data, and pansensitive and panresistant miRNAs to 21 NCCN-recommended drugs
for treating NSCLC. Italicized font indicates the gene having at least one
non-silent mutation in at least one NSCLC cell line. Underlined font indicates
the gene having both fusion and non-silent mutation in NSCLC cell lines.

| | Pansensitive | |
|---|---|---|
| drug | genes | Panresistant genes |
| afatinib | ADH7 | CCNT1, WDR47, TRIB1, hsa-miR-133a, hsa- |
| | | miR-1280 |
| alectinib | | CCNT1, hsa-miR-30b, hsa-miR-30d |
| brigatinib | | WDR47, hsa-miR-133a, hsa-miR-30a |
| cabozantinib | | hsa-miR-133a, hsa-miR-210 |
| carboplatin | | hsa-miR-210 |
| cisplatin | | CDK1, hsa-miR-133a, hsa-miR-30a, hsa-miR- |
| | | 30b |
| crizotinib | hsa-miR-223, | hsa-miR-30b, hsa-miR-30d |
| | hsa-miR-218 | |
| dabrafenib | hsa-miR-218 | GLI2, CLCN5, CDKN3 |
| dacomitinib | | GLTP, FBXL4, hsa-miR-195, hsa-miR-30a |
| docetaxel | | RAB30, NCEH1, GLTP, COPG1, |
| | | hsa-miR-133a, hsa-miR-210, hsa-miR-30a |
| erlotinib | DGKE | GLTP, hsa-miR-1280 |
| gefitinib | | hsa-miR-195 |
| gemcitabine | | CDK1, CDK16 |
| osimertinib | | WDR47, hsa-miR-30a |
| paclitaxel | | RAB30, hsa-miR-30a |
| pemetrexed | | CDK16, hsa-miR-34b |
| trametinib | | FOS, CDC42, PCBP1, TAOK2, NISCH, hsa- |
| | | miR-195 |
| vemurafenib | | hsa-miR-195 |
| vinorelbine | | FOS, CDC42, RAB30, NCEH1, GLTP, hsa-miR- |
| | | 133a, hsa-miR-30a |

The association between diagnostic miRNAs-targeted genes and the radiotherapy response was then investigated. Gene expression levels in patients with stage III or IV who underwent radiotherapy in the TCGA-LUAD and TCGA-LUSC cohorts was analyzed. Specifically, a comparison was made of the mRNA expression levels of these genes between patients with a long survival (>58 months; n=5) and short survival (<20 months; n=20) following radiotherapy. Genes that showed significant differential expression (P<0.05, two-sample t-tests) between the two groups were classified as radiotherapy-sensitive (higher expression in the long-survival group) or radiotherapy-resistant (higher expression in the short-survival group). This identified 210 genes that were either radiotherapy-sensitive or -resistant.

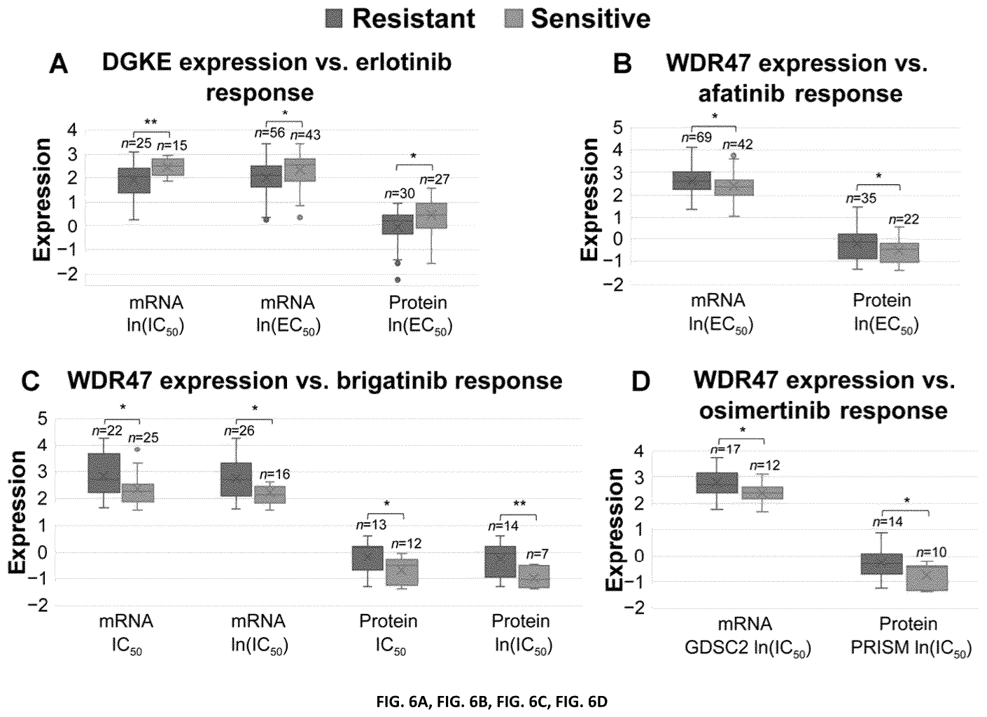

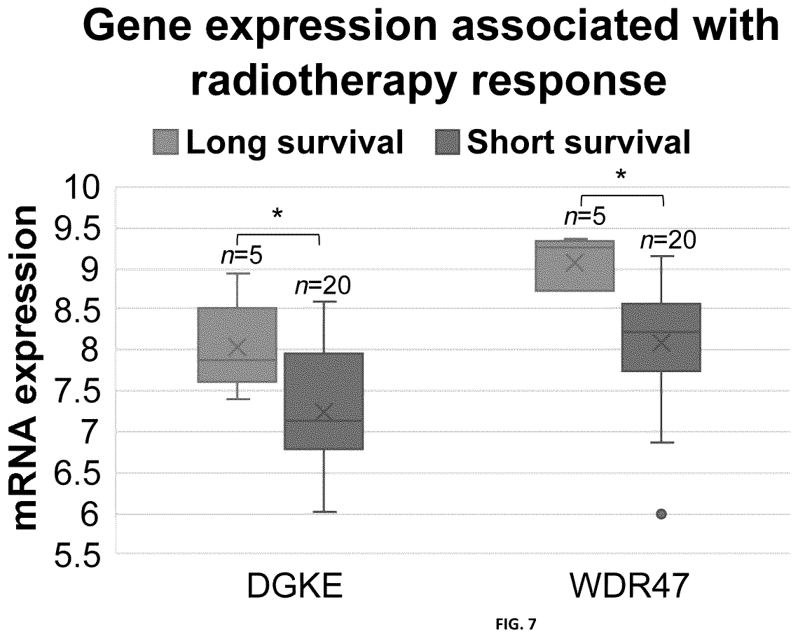

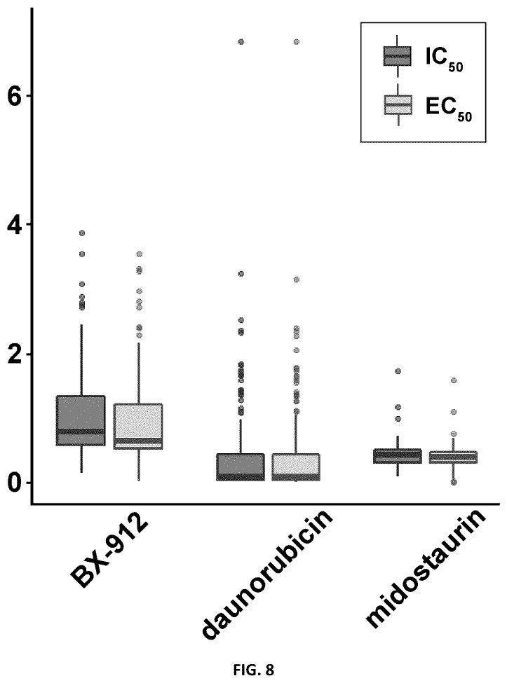

DGKE and WDR47 were found with significant associations with responses to both systemic therapies and radiotherapy (Table 3, FIGS. 6 and 7). DGKE is targeted by hsa-miR-3065-5p, and its expression was associated with sensitivity to erlotinib and radio-therapy. Hsa-miR-139-5p targets WDR47, which was resistant to afatinib, brigatinib, and osimertinib, and was sensitive to radiotherapy.

Identification of Prognostic miRNA-Targeted Genes. Using Xu's LUAD dataset, as well as TCGA-LUAD and TCGA-LUSC data to assess the prognostic significance of the identified miRNA-targeted genes. Genes that were significantly overexpressed in tumor samples in the log 2-transformed mRNA data or had a significant hazard ratio>1 in the univariate Cox model were identified as hazardous genes. Conversely, genes that were significantly underexpressed in lung cancer tumor tissues in the log 2-transformed mRNA data or had a significant hazard ratio<1 in the univariate Cox model were identified as protective genes. To be selected as mRNA prognostic genes, the genes had to be concordant in at least two of the five categories (mRNA differential expression between tumors vs. non-cancerous adjacent tissues in Xu's LUAD, as well as significant association with patient survival in Xu's LUAD, TCGA-LUAD, TCGA-LUSC, and TCGA-NSCLC).

Discovery of Repositioning Drugs. To discover connected perturbagen signatures and repositioning drugs for treating NSCLC, this study utilized the above-identified prognostic genes and proliferation genes in CRISPR-Cas9/RNAi screening assays from the miRNA-targeted genes using CMap. Two sets of genes were defined as CMap input lists. CMap input list 1 included (1) an up-regulated gene list (n=100) consisting of protective miRNA-targeted genes (defined in section 3.7), which also had a significant effect in less than 50% of the tested NSCLC cell lines in both CRISPR-Cas9 and RNAi screening assays, and (2) a down-regulated gene list (n=35) that comprised of hazardous miRNA-targeted genes (defined in section 3.7) that had a significant effect in more than 50% of the tested NSCLC cell lines in both proliferation screening assays. CMap input list 2 included an up-regulated gene list (n=85) consisting of protective miR-NA-targeted genes that had no significant effect in both proliferation screening assays in NSCLC cell lines, and a down-regulated gene list (n=79) consisting of hazardous miR-NA-targeted genes that had a significant effect in more than 50% of the NSCLC cell lines in at least one of CRISPR-Cas9 and RNAi screening assays.

Three targeted therapeutic candidates were selected with a low averaged IC50 and EC50 in human NSCLC cell lines (FIG. 8), indicating their efficacy in inhibiting NSCLC cell growth. The selected compounds were BX-912 from drug set PY-RUVATE_DEHYDROGENASE_KINASE_INHIBITOR, daunorubicin from drug set RNA_SYNTHESIS_INHIBITOR, and midostaurin from drug set CP_FLT3_INHIBITOR. These three pathways were not identified in our previous network studies for repositioning drug discovery [Ye, Q., et al., A Multi-Omics Network of a Seven-Gene Prognostic Signature for Non-Small Cell Lung Cancer. Int J Mol Sci, 2021. 23(1), Ye, Q., et al., Multi-Omics Immune Interaction Networks in Lung Cancer Tumorigenesis, Proliferation, and Survival. International Journal of Molecular Sciences, 2022. 23(23): p. 14978, Ye, Q. and N. L. Guo, Single B Cell Gene Co-Expression Networks Implicated in Prognosis, Proliferation, and Therapeutic Responses in Non-Small Cell Lung Cancer Bulk Tumors. Cancers, 2022. 14(13): p. 3123].

BX-912 is an inhibitor of 3-phosphoinositide-dependent protein kinase 1 PDK1/Akt signaling [Mashukova, A., et al., PDK1 in apical signaling endosomes participates in the rescue of the polarity complex atypical PKC by intermediate filaments in intestinal epithelia. Mol Biol Cell, 2012. 23(9): p. 1664-74]. It is an experimental drug that has been studied for its potential therapeutic use in the treatment of breast, colon, and prostate cancers, as well as gliomas [Feldman, R. I., et al., Novel small molecule inhibitors of 3-phosphoinositide-dependent kinase-1. J Biol Chem, 2005. 280(20): p. 19867-74] and mantle cell lymphoma [Maegawa, S., et al., Phosphoinositide-dependent protein kinase 1 is a potential novel therapeutic target in mantle cell lymphoma. Exp Hematol, 2018. 59: p. 72-81.e2]. Daunorubicin, belonging to the anthracyclines class, is a chemotherapy drug for treating leukemias and Kaposi's sarcoma [Saleem, T. and A. Kasi, Daunorubicin. 2020, Mayer, L. D., P. Tardi, and A. C. Louie, CPX-351: a nanoscale liposomal co-formulation of daunorubicin and cytarabine with unique biodistribution and tumor cell uptake properties. Int J Nanomedicine, 2019. 14: p. 3819-3830]. Midostaurin is a protein kinase inhibitor. It has been approved for use in the treatment of certain types of blood cancers, including acute myeloid leukemia (AML) and myelodysplastic syndrome (MDS) [Gotlib, J., et al., Efficacy and Safety of Midostaurin in Advanced Systemic Mastocytosis. N Engl J Med, 2016. 374(26): p. 2530-41, Stone, R. M., et al., Midostaurin plus Chemotherapy for Acute Myeloid Leukemia with a FLT3 Mutation. N Engl J Med, 2017. 377(5): p. 454-464]. Their potential utility in treating NSCLC was not known before,

To characterize responders of these three potential repositioning drugs, genes associated with the drug response in CCLE NSCLC cell lines were selected on a genome-wide scale (Table 4). The selected genes had a concordant significantly differential mRNA and protein expression in sensitive vs. resistant NSCLC cell lines to the specific compound in the PRISM data. None of the genes found had a significant association with radiotherapy response. Most of the genes had gene mutations and/or fusions in NSCLC cell lines.

TABLE 4
Genes associated with sensitivity/resistance to the selected compounds
with concordant mRNA and protein expression. Italicized font
indicates the gene having at least one non-silent mutation in
NSCLC cell lines. Underlined font indicates the gene having both
fusions and non-silent mutations in NSCLC cell lines.

| | | Resistant |
|---|---|---|
| Drug | Sensitive Genes | Genes |
| BX-912 | ARSD, ATG4C, CD44, CDK6, | BCAS1, |
| | CNST, G3BP2, GSTA5, | CD14, |
| | KDM6B, LGMN, MCM9, | CGNL1, |
| | NCOR2, NEGR1, NPC2, | PALM3, |
| | NT5C, PTPRG, RAB32, | VTCN1 |
| | RPTOR, RRBP1, SF3B5, | |
| | SOGA3, STAU1, TGFB2, | |
| | TMCC2 | |
| daunorubicin | BCOR, KMT2B, NUDT8, | |
| | OTUD4, SMS, SYT7 | |
| midostaurin | ACBD5, APLF, ASH1L, | |
| | BSCL2, CENPB, FMO5, JAK1, | |
| | NRCAM, OPA3, PCGF1, | |
| | SHC1, WDR53, ZHX3 | |

Discussion According to the National Institute of Cancer (NCI) data, about 55% of lung cancer cases are diagnosed when the disease has already metastasized [Cancer Stat Facts: Lung and Bronchus Cancer. [cited 2021 May 26, 2021]; Available from: https://seer.cancer.gov/statfacts/html/lungb.html]. The National Lung Screening Trial (NLST) conducted a study of 53,454 participants at high risk for lung cancer in the United States. The NLST results showed that three annual computed tomographic (CT) screenings led to a 20% lower mortality from lung cancer than screening with chest radiography after a median follow-up of 6.5 years [de Koning, H. J., et al., Reduced Lung-Cancer Mortality with Volume CT Screening in a Randomized Trial. N Engl J Med, 2020. 382(6): p. 503-513]. Nevertheless, CT screening for lung cancer has several limitations, including a high false positive rate and associated overdiagnosis and overtreatment, radiation, limited coverage for specific age and smoking history groups, and costs. According to NLST, The false positive rate of CT screening was about 96.4% [Aberle, D. R., et al., Reduced lung-cancer mortality with low-dose computed tomographic screening. N Engl J Med, 2011. 365(5): p. 395-409]. Thus, 96 out of 100 individuals who undergo CT screenings will have a false-positive result showing the presence of a lung nodule or another abnormality that is not cancer. As a con-sequence, it was reported that 9% of the patients who received lung cancer surgery did not have malignancy [Smith, M. A., et al., Prevalence of benign disease inpatients undergoing resection for suspected lung cancer. Ann Thorac Surg, 2006. 81(5): p. 1824-8; discussion 1828-9]; the benign rates in invasive biopsies for patients of suspicious lung cancer range from 20-30% [Lee, K. H., et al., Diagnostic Accuracy of Percutaneous Transthoracic Needle Lung Biopsies: A Multicenter Study. Korean J Radiol, 2019. 20(8): p. 1300-1310, Quint, L. E., et al., CT-guided thoracic core biopsies: value of a negative result. Cancer Imaging, 2006. 6(1): p. 163-7]. Hence, the development of minimally invasive blood-based diagnostic tests is an unmet clinical need to improve clinical decision-making in lung cancer treatment.

Lung cancer is a heterogeneous disease with distinct histological subtypes and complex somatic mutations. Tumor heterogeneity within disease sites contributes to resistance to cancer therapies and poses a challenge to biomarker discovery [Dagogo-Jack, I. and A. T. Shaw, Tumour heterogeneity and resistance to cancer therapies. Nat Rev Clin Oncol, 2018. 15(2): p. 81-94, McGranahan, N. and C. Swanton, Clonal Heterogeneity and Tumor Evolution: Past, Present, and the Future. Cell, 2017. 168(4): p. 613-628]. MiRNAs have emerged as important diagnostic and prognostic biomarkers and therapeutic targets in cancer treatment due to their functional involvement in gene and protein regulation and intracellular signaling [Condrat, C. E., et al., miRNAs as Biomarkers in Disease: Latest Findings Regarding Their Role in Diagnosis and Prognosis. Cells, 2020. 9(2), Rupaimoole, R. and F. J. Slack, MicroRNA therapeutics: towards a new era for the management of cancer and other diseases. Nature Reviews Drug Discovery, 2017. 16(3): p. 203-222]. MiRNA biomarkers are promising for the early detection of NSCLC supplementing low-dose CT screening [Kan, C. F. K., et al., Circulating Biomarkers for Early Stage Non-Small Cell Lung Carcinoma Detection: Supplementation to Low-Dose Computed Tomography. Front Oncol, 2021. 11: p. 555331] and the prognosis of advanced NSCLC patients treated with immunotherapy [Boeri, M., et al., Circulating miRNAs and PD-L1 Tumor Expression Are Associated with Survival in Advanced NSCLC Patients Treated with Immunotherapy: a Prospective Study. Clin Cancer Res, 2019. 25(7): p. 2166-2173].

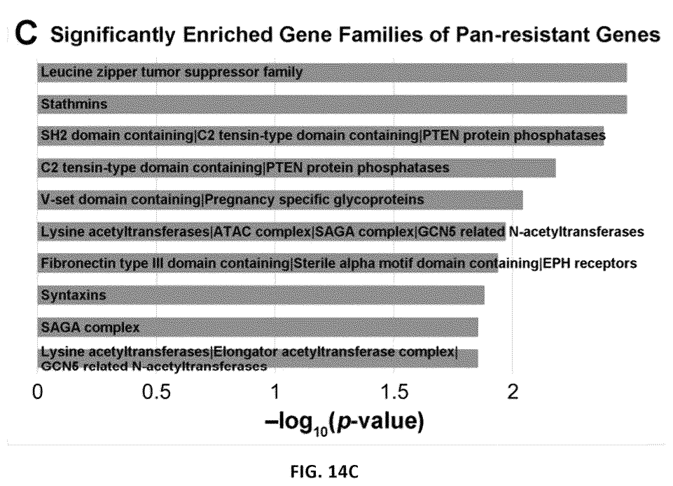

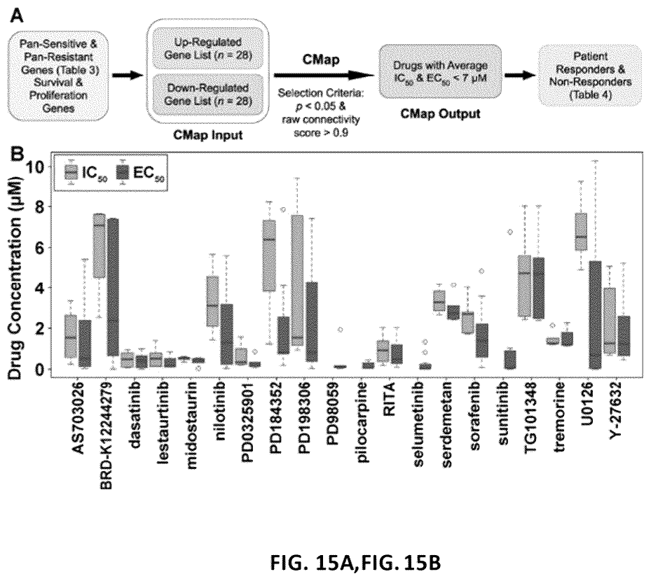

This disclosure identifies a set of 73 miRNA markers differentially expressed between normal lung tissues and lung cancer tumors. This set of 73 miRNAs can accurately distinguish lung cancer tumors from normal lung cancer tissues with an overall accuracy greater than 92% in both supervised and unsupervised classification in separate patient cohorts (n=484). Furthermore, seven miRNAs showed significant differential expression in lung cancer patient blood samples versus normal samples (FIG. 5C). These seven miRNAs can accurately separate blood samples from lung cancer patients (n=4) and normal individuals (n=6) with 100% of accuracy in unsupervised clustering, indicating its potential utility as a minimally-invasive diagnostic test. Among the seven miRNAs, hsa-miR-543 had concordant overexpression in both lung cancer tumor tissues and blood samples. Numerous miRNAs showed consistent expression patterns in NSCLC, SCLC, and carcinoid compared with normal lung tissues (FIGS. 4A, 4B). The majority of miRNAs showed consistent expression patterns between NSCLC and SCLC, but not with carcinoid when compared with normal lung tissues (FIG. 4C). MiRNA-205, miRNA-363, and miRNA-99a had opposite expression patterns between NSCLC and SCLC in comparison with normal lung tissues. Among the identified 73 miRNAs, numerous miRNAs were consistently dysregulated in lung cancer tumors vs normal lung tissues across different patient cohorts (FIGS. 5A-5D), 14 of which also had a consistent association with TCGA NSCLC patient survival outcomes (Table 2). Given the heterogeneity of lung cancer, it was acknowledged that the identified 73 diagnostic miRNAs need to be further validated in separate patient cohorts to substantiate their clinical utility. Our study showed the feasibility to extract miRNAs from blood samples and use the expression of our identified miRNAs to distinguish 4 lung cancer patients from 6 normal individuals. This disclosure plans to validate the results using blood samples collected from more patients including the NLST in our future research.

Ten miRNAs were identified as potential tumor suppressors, including hsa-miR-144, hsa-miR-195, hsa-miR-223, hsa-miR-30a, hsa-miR-30b, hsa-miR-30d, hsa-miR-335, hsa-miR-363, hsa-miR-451, and hsa-miR-99a. Four miRNAs were identified as potential oncogenic, including hsa-miR-21, hsa-miR-31, hsa-miR-411, and hsa-miR-494 (Table 2). Hsa-miR-144 functions as a tumor suppressor by inhibiting epithelial-to-mesenchymal transition (EMT), proliferation, migration, invasion, metastasis, and angiogenesis in multiple human cancers [Pan, Y., et al., miR-144 functions as a tumor suppressor in breast cancer through inhibiting ZEB1/2-mediated epithelial mesenchymal transition process. Onco Targets Ther, 2016. 9: p. 6247-6255, Yin, Y., et al., MiR-144 suppresses proliferation, invasion, and migration of breast cancer cells through inhibiting CEP55. Cancer Biology & Therapy, 2018. 19(4): p. 306-315, Kooshkaki, O., et al., MiR-144: A New Possible Therapeutic Target and Diagnostic/Prognostic Tool in Cancers. Int J Mol Sci, 2020. 21(7), Sheng, S., et al., MiR-144 inhibits growth and metastasis in colon cancer by down-regulating SMAD4. Biosci Rep, 2019. 39(3), Sun, X. B., et al., MicroRNA-144 Suppresses Prostate Cancer Growth and Metastasis by Targeting EZH2. Technol Cancer Res Treat, 2021. 20: p. 1533033821989817, Wu, M., et al., MicroRNA-144-3p suppresses tumor growth and angiogenesis by targeting SGK3 in hepatocellular carcinoma. Oncol Rep, 2017. 38(4): p. 2173-2181, Gu, J., et al., MicroRNA-144 inhibits cell proliferation, migration and invasion in human hepatocellular carcinoma by targeting CCNB1. Cancer Cell Int, 2019. 19: p. 15, Chen, S., et al., MiR-144 inhibits proliferation and induces apoptosis and autophagy in lung cancer cells by targeting TIGAR. Cell Physiol Biochem, 2015. 35(3): p. 997-100756-63]. Synergistically, the miR-144/451a cluster inhibits metastasis [Zhang, J., et al., Transcriptional control of PAX4-regulated miR-144/451 modulates metastasis by suppressing ADAMs expression. Oncogene, 2015. 34(25): p. 3283-95] and is a tumor suppressor in oral squamous cell carcinoma through the inhibition of cancer cell invasion, migration, and clonogenic potential [Manasa, V. G., S. Thomas, and S. Kannan, MiR-144/451a cluster synergistically modulates growth and metastasis of Oral Carcinoma. Oral Dis, 2023. 29(2): p. 584-594], with potential therapeutic applications using biomimetic nanosystems [Li, K., et al., Biomimetic Nanosystems for the Synergistic Delivery of miR-144/451a for Oral Squamous Cell Carcinoma. Balkan Med J, 2022. 39(3): p. 178-186]. Downregulation of miR-195 was reported in multiple human cancer types and linked to its tumor-suppressive or oncogenic functional roles [Yu, W., et al., MicroRNA-195: a review of its role in cancers. Onco Targets Ther, 2018. 11: p. 7109-7123]. MiR-195 was panresistant to 21 NCCN-recommended NSCLC drugs (Table 3). MiR-233 controls innate immune responses to maintain the homeostasis of myeloid cells [Yuan, X., et al., MicroRNA miR-223 as regulator of innate immunity. J Leukoc Biol, 2018. 104(3): p. 515-524] and blocks cell-cyle progression in myeloid cells [Pulikkan, J. A., et al., Cell-cycle regulator E2F1 and microRNA-223 comprise an autoregulatory negative feedback loop in acute myeloid leukemia. Blood, 2010. 115(9): p. 1768-78] functioning through negative feedback loops. MiR-233 inhibits the proliferation and metastasis of oral squamous cell carcinoma [Sun, C., X. H. Liu, and Y. R. Sun, MiR-223-3p inhibits proliferation and metastasis of oral squamous cell carcinoma by targeting SHOX2. Eur Rev Med Pharmacol Sci, 2019. 23(16): p. 6927-6934] and NSCLC [Dou, L., et al., miR-223-5p Suppresses Tumor Growth and Metastasis in Non-Small Cell Lung Cancer by Targeting E2F8. Oncol Res, 2019. 27(2): p. 261-268]. In other contexts, miR-233 was upregulated in pancreatic cancer, gastric cancer, and ovarian cancer, and promoted cancer cell proliferation, migration, and invasion [Fang, G., et al., MicroRNA-223-3p Regulates Ovarian Cancer Cell Proliferation and Invasion by Targeting SOX11 Expression. Int J Mol Sci, 2017. 18(6), Ma, L., et al., Increased microRNA-223 in Helicobacter pylori-associated gastric cancer contributed to cancer cell proliferation and migration. Biosci Biotechnol Biochem, 2014. 78(4): p. 602-8, Haneklaus, M., et al., miR-223: infection, inflammation and cancer. J Intern Med, 2013. 274(3): p. 215-26]. MiR-30a functions as a tumor suppressor in colon cancer [Baraniskin, A., et al., MiR-30a-5p suppresses tumor growth in colon carcinoma by targeting DTL. Carcinogenesis, 2012. 33(4): p. 732-9], hepatocellular carcinoma [He, R., et al., MiR-30a-5p suppresses cell growth and enhances apoptosis of hepatocellular carcinoma cells via targeting AEG-1. Int J Clin Exp Pathol, 2015. 8(12): p. 15632-41], and breast cancer [Tang, J., A. Ahmad, and F. H. Sarkar, The Role of MicroRNAs in Breast Cancer Migration, Invasion and Metastasis. International Journal of Molecular Sciences, 2012. 13(10): p. 13414-13437]. MiR-30b suppresses cancer cell growth, migration, and invasion in esophageal cancer [Li, Q., et al., miR-30b inhibits cancer cell growth, migration, and invasion by targeting homeobox A1 in esophageal cancer. Biochem Biophys Res Commun, 2017. 485(2): p. 506-512] and papillary thyroid cancer [Wang, Y., et al., miR-30b-5p inhibits proliferation, invasion, and migration of papillary thyroid cancer by targeting GALNT7 via the EGFR/PI3K/AKT pathway. Cancer Cell International, 2021. 21(1): p. 618]. MiR-30d suppresses NSCLC tumor invasion and migration by targeting Nuclear factor I B (NFIB) [Wu, Y., et al., Non-small cell lung cancer: miR-30d suppresses tumor invasion and migration by directly targeting NFIB. Biotechnol Lett, 2017. 39(12): p. 1827-1834]. Although the tumor-suppressive roles of the miR-30 family have been reported in many human cancers including lung cancer [Kanthaje, S., et al., Repertoires of MicroRNA-30family as gate-keepers in lung cancer. FBS, 2021. 13(2): p. 141-156], miR-30 can also disrupt senescence and promote cancer through the inhibition of p16INK4A and p53 [Su, W., et al., miR-30 disrupts senescence and promotes cancer by targeting both p16(INK4A) and DNA damage pathways. Oncogene, 2018. 37(42): p. 5618-5632]. This analysis showed that the miR-30 family is pan-resistant to 21 NCCN-recommended drugs for treating NSCLC (Table 3). MiR-335 suppresses the proliferation of NSCLC cells by targeting Tra2β [Liu, J., et al., miR-335 inhibited cell prohferation of lung cancer cells by target Tra2β. Cancer Sci, 2018. 109(2): p. 289-296] and CCNB2 [Wang, X., et al., miR-335-5p Regulates Cell Cycle and Metastasis in Lung Adenocarcinoma by Targeting CCNB2. Onco Targets Ther, 2020. 13: p. 6255-6263]. MiR-363-3p inhibits proliferation and colony formation by targeting PCNA in lung adenocarcinoma A549 and H441 cells [Wang, Y., et al., miR-363-3p inhibits tumor growth by targeting PCNA in lung adenocarcinoma. Oncotarget, 2017. 8(12): p. 20133-20144]. MiR-363-3p suppresses migration, invasion, and EMT by targeting NEDD9 and SOX4 in NSCLC [Chang, J., et al., miR-363-3p inhibits migration, invasion, and epithelial-mesenchymal transition by targeting NEDD9 and SOX4 in non-small-cell lung cancer. J Cell Physiol, 2020. 235(2): p. 1808-1820]. MiR-451 inhibits NSCLC tumor cell growth and migration by targeting LKB1/AMPK [Liu, Y., et al., Mir-451 inhibits proliferation and migration of non-small cell lung cancer cells via targeting LKB1/AMPK. Eur Rev Med Pharmacol Sci, 2019. 23(3 Suppl): p. 274-280] and ATF2 [Shen, Y. Y., et al., MiR-451a suppressed cell migration and invasion in non-small cell lung cancer through targeting ATF2. Eur Rev Med Pharmacol Sci, 2018. 22(17): p. 5554-5561] and sensitizes NSCLC cells to cisplatin by regulating Mcl-1 [Cheng, D., et al., MicroRNA-451 sensitizes lung cancer cells to cisplatin through regulation of Mcl-1. Mol Cell Biochem, 2016. 423(1-2): p. 85-91]. MiR-99a suppresses EMT and stemness through the inhi-bition of two oncogenic proteins E2F2 and EMR2 [Feliciano, A., et al., miR-99a reveals two novel oncogenic proteins E2F2 and EMR2 and represses stemness in lung cancer. Cell Death Dis, 2017. 8(10): p. e3141] and enhances radiation sensitivity by targeting mTOR in NSCLC [Yin, H., et al., MiR-99a Enhances the Radiation Sensitivity of Non-Small Cell Lung Cancer by Targeting mTOR. Cell Physiol Biochem, 2018. 46(2): p. 471-481]. All the evidence in the literature confirmed the 10 putative tumor suppressive miRNAs identified in our study. MiR-21 is a well-recognized oncogene in many human cancers, including NSCLC [92]. MiR-31 is also a prominent oncogene in lung cancer. Overexpression of miR-31 was found in major histological subtypes of NSCLC, but not in SCLC or carcinoid in a published study [Bica-Pop, C., et al., Overview upon miR-21 in lung cancer: focus on NSCLC. Cell Mol Life Sci, 2018. 75(19): p. 3539-3551]. In this patient cohort, significant overexpression of miR-31 and miR-31* was found in NSCLC and SCLC, but the results were not statistically significant in carcinoid when compared with normal lung tissues (FIG. 4A). MiR-411 is oncogenic in lung cancer through the miR-411-SPRY4-AKT axis [Zhang, C., et al., Oncogenic microRNA-411 promotes lung carcinogenesis by directly targeting suppressor genes SPRY4 and TXNIP. Oncogene, 2019. 38(11): p. 1892-1904]. Tumor-drived miR-494 promotes angiogenesis in NSCLC under hypoxic conditions [Mao, G., et al., Tumor-derived microRNA-494 promotes angiogenesis in non-small cell lung cancer. Angiogenesis, 2015. 18(3): p. 373-82]. CircVAPA accelerates SCLC progression through the miR-377-3p and miR-494-3p/IGF1R/AKT axis [Hua, J., et al., CircVAPA promotes small cell lung cancer progression by modulating the miR-377-3p and miR-494-3p/IGF1/AKT axis. Mol Cancer, 2022. 21(1): p. 123]. All the published results confirmed the four oncogenic miRNAs identified in this study.

To decipher miRNA-mediated molecular machinery and determine the clinical relevance in NSCLC, this disclosure identified experimentally validated target genes of 73 miRNAs using TarBase. From these target genes, this disclosure identified pansensitive or panresistant genes to 21 NCCN-recommended drugs for treating NSCLC concordant at both mRNA and protein expression levels (Table 3). Genes associated with radiotherapy response in TCGA NSCLC patients were also identified. Among pansensitive genes, DGKE mRNA and protein expression was associated with sensitivity to erlotinib and was not associated with resistance to any of the 21 NCCN-recommended drugs (FIG. 6A). Among panresistant genes, WDR47 mRNA and protein expression was associated with resistance to afatinib, brigatinib, and osimertinib (FIGS. 6B-D). DGKE and WDR47 were both associated with radiotherapy sensitivity (FIG. 7). Non-silent mutations of DGKE and WDR47 were found in NSCLC cell lines. Recessive mutations in DGKE cause small-vessel thrombosis and kidney failure [Lemaire, M., et al., Recessive mutations in DGKE cause atypical hemolytic-uremic syndrome. Nat Genet, 2013. 45(5): p. 531-6]. The silenced status of DGKE was found in nonadherent human colon cancer HT-29 cells [Marquez, J., et al., Identification of hepatic microvascular adhesion-related genes of human colon cancer cells using random homozygous gene perturbation. Int J Cancer, 2013. 133(9): p. 2113-22]. DGKE was targeted by hsa-miR-3065-5p, which was overexpressed in colon cancer [Martinez-Gutierrez, A., et al., A microRNA panel that regulates proinflammatory cytokines as diagnostic and prognosis biomarkers in colon cancer. Biochemistry and Biophysics Reports, 2022. 30: p. 101252]. In this study, hsa-miR-3065-5p was underexpressed in lung cancer tumors. WDR47 plays an important role in microtubule formation [Liu, H., et al., Wdr47, Camsaps, and Katanin cooperate to generate ciliary central microtubules. Nat Commun, 2021. 12(1): p. 5796]. WDR47 is targeted by hsa-miR-139-5p, which was found to be underexpressed in lung cancer tumors in this study. Our results provided new insight into hsa-miR-3065-5p/DGKE and hsa-miR-139-5p/WDR47 in NSCLC. Further experimental investigation of these two genes will be conducted in our future research.

Based on the identified miRNA-mediated transcriptional networks in NSCLC, this disclosure sought to discover new drugs or new indications of existing drugs for treating NSCLC that were not known before. Specifically, among the target genes of the 73 diagnostic miRNAs, protective genes were selected as associated with favorable patient outcomes and underexpressed in lung cancer tumors; hazard genes were selected as associated with poor patient outcomes and overexpressed in lung cancer tumors. Proliferation genes were also selected from CRISPR-Cas9/RNAi screening data in NSCLC cell lines. Candidate compounds that can upregulate protective genes and downregulate hazard genes and proliferation genes were identified using CMap. Next, therapeutic compounds that are effective in inhibiting NSCLC cell growth are pinpointed from the candidate compounds. New indications for treating NSCLC were discovered for three drugs in this study, including BX-912, daunorubicin, and midostaurin. These findings provided evidence to design future clinical trials to test the efficacy of these three compounds as repositioning drugs in treating NSCLC. Most clinical trials failed because drug responders were not well-characterized. To characterize responders of these three potential repositioning drugs, genes associated with the response to these compounds were selected on a genome-wide scale in CCLE NSCLC cell lines (Table 4). The selected genes had a concordant significantly differential mRNA and protein expression in sensitive vs. resistant NSCLC cell lines to the specific compound. The majority of these genes had non-silent mutations and/or gene fusions in the examined NSCLC cell lines. The results presented in this study will accelerate the design of future clinical trials and expedite the R&D of repositioning drugs to improve lung cancer patient survival outcomes.

Conclusions. This study identified a set of 73 miRNAs for the accurate detection of lung cancer tumors from normal lung tissues. Seven miRNAs were also identified to classify blood samples from lung cancer patients from healthy individuals. Combined with survival analysis of TCGA NSCLC patients, 10 miRNAs were identified as potential tumor suppressors, and 4 as potential oncogenes, supported by their reported roles in published literature. From experimentally validated target genes of these 73 miRNAs, DGKE and WDR47 were found with significant associations with responses to both systemic therapies and radiotherapy in NSCLC. Based on our identified miRNA-mediated molecular network, BX-912, daunorubicin, and midostaurin were discovered as potential repositioning drugs for treating NSCLC.

### Breast Cancer

Breast cancer treatment can be improved with biomarkers for early detection and individualized therapy. A set of 86 microRNAs (miRNAs) were identified to separate breast cancer tumors from normal breast tissues (n=52) with an overall accuracy of 90.4%. Six miRNAs had concordant expression in both tumors and breast cancer patient blood samples compared with the normal control samples. Twelve miRNAs showed concordant expression in tumors vs. normal breast tissues and patient survival (n=1,093), with seven as potential tumor suppressors and five as potential oncomiRs. Frorn experimentally validated target genes of these 86 miRNAs, pan-sensitive and pan-resistant genes with concordant mRNA and protein expression associated with in-vitro drug response to 19 NCCN-recommended breast cancer drugs were selected. Combined with in-vitro proliferation assays using CRISPR-Cas9/RNAi and patient survival analysis, MEK inhibitors PD19830 and BRD-K12244279, pilocarpine, and tremorine were discovered as potential new drug options for treating breast cancer. Multi-omics biomarkers of response to the discovered drugs were identified using human breast cancer cell lines. This study presented an artificial intelligence pipeline of miRNA-based discovery of biomarkers, therapeutic targets, and repositioning drugs that can be applied to many cancer types.

MicroRNAs (miRNAs) are a class of small non-coding RNAs that regulate gene expression and activate translation under certain circumstances. The dynamic interaction between miRNAs and their target genes is dependent on numerous factors, including miRNA subcellular location, target mRNA abundance, and miRNA-mRNA interaction affinity [O'Brien, J.; Hayder, H.; Zayed, Y.; Peng, C. Overview of MicroRNA Biogenesis, Mechanisms of Actions, and Circulation. Frontiers in endocrinology 2018, 9, doi:10.3389/fendo.2018.00402.]. In addition to miRNA-target mRNA dynamics, alterations in miRNA-induced silencing complex (miRISC) localization in response to fluctuations in cellular environments, such as stress induced by heat shock and translation inhibition and serum starvation, can also affect miRNA activity and intracellular miRNA levels [Kucherenko, M. M.; Shcherbata, H. R. miRNA targeting and alternative splicing in the stress response-events hosted by membrane-less compartments. Journal of cell science 2018, 131, jcs202002, Wang, K.; Zhang, S.; Weber, J.; Baxter, D.; Galas, D. J. Export of microRNAs and microRNA-protective protein by mammalian cells. Nucleic acids research 2010, 38, 7248-7259.]. Extracellular miRNAs, enclosed and transported by exosomes, mediate cell-cell communication [Valadi, H.; Ekstrom, K.; Bossios, A.; Sjostrand, M.; Lee, J. J.; Lotvall, J. O. Exosome-mediated transfer of mRNAs and microRNAs is a novel mechanism of genetic exchange between cells. Nature cell biology 2007, 9, 654-659, doi:10.1038/ncb1596.]. Both intracellular and extracellular miRNAs are pivotal in bio-genesis, cellular signaling cascades, and numerous human diseases including cancer [Bartel, D. P. Metazoan MicroRNAs. Cell 2018, 173, 20-51, doi:10.1016/j.cell.2018.03.006, Wang, M.; Yu, F.; Ding, H.; Wang, Y.; Li, P.; Wang, K. Emerging Function and Clinical Values of Exosomal MicroRNAs in Cancer. Molecular therapy. Nucleic acids 2019, 16, 791-804, doi:10.1016/j.omtn.2019.04.027.].

The investigation of miRNA expression and its implications in carcinogenesis has garnered significant attention within the field of cancer biology. For instance, several cancer-related pathways are regulated by miRNA, including apoptosis, differentiation, proliferation, and stem cell maintenance [Farazi, T. A.; Spitzer, J. I.; Morozov, P.; Tuschl, T. miRNAs in human cancer. The Journal of pathology 2011, 223, 102-115.]. The utilization of miRNAs as biomarkers in cancer diagnostics has gained substantial attention, holding potential superiority over mRNAs in this context. First, the distinct miRNA expression profiles observed in different tissues and tumor types highlight the significant role they play in cancer diagnostics [Ferracin, M.; Veronese, A.; Negrini, M. Micromarkers: miRNAs in cancer diagnosis and prognosis. Expert review of molecular diagnostics 2010, 10, 297-308.]. For example, Iorio et al. [Iorio, M. V.; Croce, C. M. microRNA involvement in human cancer. Carcinogenesis 2012, 33, 1126-1133.] identified a set of 15 miRNAs that were able to distinguish normal breast tissue from breast tumor samples with 100% accuracy. Researchers also noted differential miRNA expression based on various clinicopatho-logic features of the breast tumor samples such as estrogen and progesterone receptor status, positive lymph node metastasis, or higher proliferation indices. Additionally, researchers were able to classify the estrogen receptor status in 93 primary breast tumor samples [Blenkiron, C.; Goldstein, L. D.; Thorne, N. P.; Spiteri, I.; Chin, S.-F.; Dunning, M. J.; Barbosa-Morais, N. L.; Teschendorff, A. E.; Green, A. R.; Ellis, I. O. MicroRNA expression profiling of human breast cancer identifies new markers of tumor subtype. Genome biology 2007, 8, 1-16.]. Using this miRNA expression data and an independent test set of samples, this study also was able to classify basal-like and luminal A tumors according to their molecular subtype classification. Second, miRNAs can be isolated from a variety of samples in which mRNA extraction is usually difficult or unsuccessful such as forma-lin-fixed paraffin-embedded samples and serum or plasma samples. [Iorio, M. V.; Ferracin, M.; Liu, C.-G.; Veronese, A.; Spizzo, R.; Sabbioni, S.; Magri, E.; Pedriali, M.; Fabbri, M.; Campiglio, M. MicroRNA gene expression deregulation in human breast cancer. Cancer research 2005, 65, 7065-7070, Nelson, P. T.; Baldwin, D. A.; Scearce, L. M.; Oberholtzer, J. C.; Tobias, J. W.; Mourelatos, Z. Microarray-based, high-throughput gene expression profiling of microRNAs. Nature methods 2004, 1, 155-161, Shingara, J.; Keiger, K.; Shelton, J.; Laosinchai-Wolf, W.; Powers, P.; Conrad, R.; Brown, D.; Labourier, E. An optimized isolation and labeling platform for accurate microRNA expression profiling. Rna 2005, 11, 1461-1470]. A unique feature of miRNAs is their presence in biofluids, such as serum, plasma, saliva, and amniotic fluid [Cortez, M. A.; Bueso-Ramos, C.; Ferdin, J.; Lopez-Berestein, G.; Sood, A. K.; Calin, G. A. MicroRNAs in body fluids—the mix of hormones and biomarkers. Nature reviews. Clinical oncology 2011, 8, 467-477, doi:10.1038/nrclinonc.2011.76.]. A set of 26 miRNAs were identified in plasma samples that can separate normal and breast cancer patients [Zhao, H.; Shen, J.; Medico, L.; Wang, D.; Ambrosone, C. B.; Liu, S. A pilot study of circulating miRNAs as potential biomarkers of early stage breast cancer. PloS one 2010, 5, e13735]. Multiple breast cancer-derived exo-somal miRNAs showed promise as liquid biopsy biomarkers to predict metastasis [Baldasici, O.; Pileczki, V.; Cruceriu, D.; Gavrilas, L. I.; Tudoran, O.; Balacescu, L.; Vlase, L.; Balacescu, O. Breast Cancer-Delivered Exosomal miRNA as Liquid Biopsy Biomarkers for Metastasis Prediction: A Focus on Translational Research with Clinical Applicability. International journal of molecular sciences 2022, 23, doi:10.3390/ijms23169371].

A single miRNA can regulate a broad spectrum of target protein-coding genes and subsequently direct entire cellular signaling pathways. Thus, modest changes in miRNAs can affect complex genetic, transcriptional, and translational networks, offering efficient therapeutic approaches in addition to diagnostic potential [Huang, W. MicroRNAs: biomarkers, diagnostics, and therapeutics. Bioinformatics in MicroRNA research 2017, 57-6]. Despite the emerging opportunities, miRNA-based drugs need to overcome several technical difficulties, including the selection of appropriate delivery routes, management of in-vivo stability, targeting of specific tissues and cell types, and achievement of the desired intracellular effects [Diener, C.; Keller, A.; Meese, E. Emerging concepts of miRNA therapeutics: from cells to clinic. Trends in Genetics 2022, 38, 613-626, doi:10.1016/j.tig.2022.02.006]. Due to these practical challenges, there are currently no miRNA drugs for treating breast cancer. Efficient artificial intelligence (AI)/machine learning (ML) pipelines are needed to utilize miRNA/mRNA/protein expression data for effective screening of chemical compounds to identify new and repositioning drugs for breast cancer treatment.

The objective of this study is to identify relevant miRNAs shown to be differentially expressed in samples from breast cancer patients versus those from normal patients. By employing bioinformatics tools and statistical analyses, we sought to deter-mine a select group of miRNA biomarkers that may be clinically useful in the diagnosis and prognosis of breast cancer. From the set of identified miRNA biomarkers, their experimentally validated target genes were selected for further analysis. Among these miRNAs and their target genes, pan-sensitive and pan-resistant biomarkers to 19 National Comprehensive Cancer Network (NCCN)-recommended drugs for treating breast cancer were identified. The proliferation potential of the target genes was assessed using public CRISPR-Cas9/RNAi screening data in Cancer Cell Line Encyclopedia (CCLE) human breast cancer cells. New and repositioning drugs were discovered for treating breast cancer based on miRNA-regulated mRNA expression signature using Connectivity Map (CMap) [Subramanian, A.; Narayan, R.; Corsello, S. M.; Peck, D. D.; Natoli, T. E.; Lu, X.; Gould, J.; Davis, J. F.; Tubelli, A. A.; Asiedu, J. K.; et al. A Next Generation Connectivity Map: L1000 Platform and the First 1,000,000 Profiles. Cell 2017, 171, 1437-1452.e1417, doi:10.1016/j.cell.2017.10.049., Lamb, J.; Crawford, E. D.; Peck, D.; Modell, J. W.; Blat, I. C.; Wrobel, M. J.; Lerner, J.; Brunet, J. P.; Subramanian, A.; Ross, K. N.; et al. The Connectivity Map: using gene-expression signatures to connect small molecules, genes, and disease. Science (New York, N.Y.) 2006, 313, 1929-1935, doi:10.1126/science.1132939]. Patient responders and non-responders to these potential new drug options were characterized by concordant mRNA/protein expression, non-silent mutations, and gene fusions in the genome-scale analysis of CCLE human breast cancer cell lines.

### Materials and Methods

Patient Samples The patient tissue samples consisted of snap-frozen breast tumors and normal breast tissue samples, which were stored at a temperature of −80° C. Frozen patient blood samples collected in EDTA blood tubes were also stored at −80° C. until further use. All patient identifying information was removed from the pathology reports of the samples while retaining important details such as cancer stage, tumor grade, and histology. West Virginia University (WVU) Tissue Bank (Morgantown, WV), WVU Mary Babb Randolph Cancer Center (MBRCC) Biorepository, and the Cooperative Human Tissue Network (CHTN) were the providers of the de-identified patient tissue samples for this study. Comprehensive patient clinical information can be found in Table 5.

TABLE 5
Clinical information of patient samples used in this study.

| | |
|---|---|
| Clinical Information | Sample size (N) |

Cancer Stage

| | |
|---|---|
| Ductal Carcinoma In Situ (DCIS) | 1 |
| 1 | 8 |
| 2 | 22 |
| 3 | 12 |
| 4 | 2 |
| Normal | 7 |

Age

| | |
|---|---|
| <60 | 27 |
| 60-80 | 17 |
| >80 | 3 |
| Missing | 5 |

Tumor Grade

| | |
|---|---|
| 1 | 3 |
| 2 | 13 |
| 3 | 25 |
| Missing | 11 |

Histology

| | |
|---|---|
| Ductal Adenocarcinoma | 1 |
| In Situ carcinoma | 1 |
| Invasive ductal carcinoma | 35 |
| Invasive lobular carcinoma | 3 |
| Metaplastic carcinoma | 1 |
| Mucinous carcinoma | 1 |
| Normal Tissue | 7 |
| Predominantly Lobular | 1 |
| Spindle Cell Sarcoma | 1 |
| Missing | 1 |

A published patient cohort from Iorio et al. [Iorio, M. V.; Croce, C. M. microRNA involvement in human cancer. Carcinogenesis 2012, 33, 1126-1133.] was used as an external validation set. This cohort contained 368 miRNA profiles quantified with microarray chips (KCl version 1.0) of seven normal breast tissue samples and 76 neoplastic breast tissue samples. The raw miRNA microarray data were available at EMBL's European Bioinformatics Institute (EBI) with the accession number E-TABM-23.

RNA Isolation and Quality Assessment From the frozen breast cancer tumors and normal breast tissue samples, total RNA was extracted using the mirVana miRNA Isolation kit following the manufacturer's in-itial protocol. From the frozen blood samples, total RNA was isolated using a modified PAXgene protocol as described by Beekman et al [Beekman, J. M.; Reischl, J.; Henderson, D.; Bauer, D.; Ternes, R.; Pena, C.; Lathia, C.; Heubach, J. F. Recovery of microarray-quality RNA from frozen EDTA blood samples. J Pharmacol ToxicolMethods 2009, 59, 44-49, doi:10.1016/j.vascn.2008.10.003]. The thawed blood samples were transferred to PAXgene blood collection tubes, and total RNA extraction was performed using the PAXgene Blood miRNA kit according to the manufacturer's instructions. The concentration of RNA was determined using the NanoDrop 1000 Spectrophotometer, while the quality of RNA was assessed using the 2100 Bioanalyzer. In the further analysis of this study, samples that met the quality control criteria were selected, including 52 tissue samples and 9 blood samples from the patients.

Microarray Analysis The miRNA profiling with additional quality controls was conducted by Ocean Ridge Biosciences utilizing custom microarrays specifically designed with 1,087 human miRNA probes. These miRNA arrays encompassed all 1,098 human miRNAs documented in the Sanger Institute mirBASE version 15. The miRNA profiling analysis incorporated various quality control features, including negative controls, specificity controls, and spiking probes. To ensure the reliability of the data, a detection threshold was determined for each array by calculating the sum of five times the standard deviation of the background signal and the 10% trim mean of the negative control probes. Probes exhibiting consistently low signals were eliminated from the subsequent statistical analysis using these threshold values. The raw and processed miRNA profiles with the aforementioned normalization method were deposited to the NCBI Gene Expression Omnibus (GEO) with the accession number GSE37963.

The Cancer Genome Atlas (TCGA) Breast Cancer Patient Cohort. Prognostic analysis involving miRNA and mRNA data was performed on the breast cancer (BRCA) patient cohort obtained from The Cancer Genome Atlas (TCGA). The datasets were procured from the LinkedOmics database [Vasaikar, S. V.; Straub, P.; Wang, J.; Zhang, B. LinkedOmics: analyzing multiomics data within and across 32 cancer types. Nucleic Acids Res 2018, 46, D956-D963, doi:10.1093/nar/gkx1090.] (http://www.linkedomics.org/, accessed on 23 May 2023). This study used the normalized mRNA RSEM data comprising 1,093 patient samples profiled with the Illumina HiSeq platform. Two sets of gene-level miRNA data were utilized: one derived from the Illumina Genome Analyzer (GA) platform (n=324), and the other from the Illumina HiSeq platform (n=755). Both miRNA datasets consisted of log 2 normalized RPM values.

Proliferation Assays. Genes functionally involved in breast cancer cell proliferation were identified from CRISPR-Cas9 knockout and RNAi knockdown screening data. The whole-genome CRISPR-Cas9 screening data of human breast cancer cell lines (n=48) were obtained from the DepMap 22Q4 data release [Dempster, J. M. R., J.; Kazachkova, M.; Pan, J.; Kugener, G.; Root, D. E.; Tsherniak, A. Extracting Biological Insights from the Project Achilles Genome-Scale CRISPR Screens in Cancer Cell Lines. bioRxiv 2019, Meyers, R. M.; Bryan, J. G.; McFarland, J. M.; Weir, B. A.; Sizemore, A. E.; Xu, H.; Dharia, N. V.; Montgomery, P. G.; Cowley, G. S.; Pantel, S.; et al. Computational correction of copy number effect improves specificity of CRISPR-Cas9 essentiality screens in cancer cells. Nature genetics 2017, 49, 1779-1784, doi:10.1038/ng.3984, Behan, F. M.; Iorio, F.; Picco, G.; Goncalves, E.; Beaver, C. M.; Migliardi, G.; Santos, R.; Rao, Y.; Sassi, F.; Pinnelli, M.; et al. Prioritization of cancer therapeutic targets using CRISPR-Cas9 screens. Nature 2019, 568, 511-516, doi:10.1038/s41586-019-1103-9] (https://depmap.org/portal, accessed on 23 May 2023). Additionally, RNAi screening data of human breast cancer cell lines (n=34) were acquired from the project Achilles (https://depmap.org/R2-D2/, accessed on 23 May 2023). In this study, a normalized dependency score of less than −0.5 in either the CRISPR-Cas9 or RNAi screening was considered indicative of a significant knock-out/knockdown effect.

Cancer Cell Line Encyclopedia (CCLE) Genome-scale protein expression data of human breast cancer cell lines (n=31) were obtained from the Gygi lab [Nusinow, D. P.; Szpyt, J.; Ghandi, M.; Rose, C. M.; McDonald, E. R., 3rd; Kalocsay, M.; Jané-Valbuena, J.; Gelfand, E.; Schweppe, D. K.; Jedrychowski, M.; et al. Quantitative Proteomics of the Cancer Cell Line Encyclopedia. Cell 2020, 180, 387-402.e316, doi:10.1016/j.cell.2019.12.023](https://gygi.hms.harvard.edu/publications/ccle.html, accessed on 25 May 2023). These data were log 2 transformed, and their mean expression levels were centered at 0. The mRNA sequencing data of breast cancer cell lines (n=63) were acquired from the DepMap 22Q4 data release [30-32] (https://depmap.org/portal, accessed on 23 May 2023), and were also log 2-transformed. Furthermore, gene mutations (n=41,707) and fusion data (n=2,979) were retrieved from the DepMap 22Q4 data release.

Drug Sensitivity Data of Breast Cancer Cell Lines The drug sensitivity data of breast cancer cell lines were obtained from two distinct sources. The first source was the Profiling Relative Inhibition Simultaneously in Mixtures (PRISM) [Corsello, S. M.; Nagari, R. T.; Spangler, R. D.; Rossen, J.; Kocak, M.; Bryan, J. G.; Humeidi, R.; Peck, D.; Wu, X.; Tang, A. A.; et al. Discovering the anti-cancer potential of non-oncology drugs by systematic viability profiling. Nature cancer 2020, 1, 235-248, doi:10.1038/s43018-019-0018-6] secondary screen data, which was provided in the DepMap 19Q4 data release (https://depmap.org/portal, accessed on 23 May 2023). The second source encompassed the Genomics of Drug Sensitivity in Cancer (GDSC1 and GDSC2) datasets [Yang, W.; Soares, J.; Greninger, P.; Edelman, E. J.; Lightfoot, H.; Forbes, S.; Bindal, N.; Beare, D.; Smith, J. A.; Thompson, I. R.; et al. Genomics of Drug Sensitivity in Cancer (GDSC): a resource for therapeutic biomarker discovery in cancer cells. Nucleic acids research 2013, 41, D955-961, doi:10.1093/nar/gksllll, Iorio, F.; Knijnenburg, T. A.; Vis, D. J.; Bignell, G. R.; Menden, M. P.; Schubert, M.; Aben, N.; Goncalves, E.; Barthorpe, S.; Lightfoot, H.; et al. A Landscape of Pharmacogenomic Interactions in Cancer. Cell 2016, 166, 740-754, doi:10.1016/j.cell.2016.06.017, Garnett, M. J.; Edelman, E. J.; Heidorn, S. J.; Greenman, C. D.; Dastur, A.; Lau, K. W.; Greninger, P.; Thompson, I. R.; Luo, X.; Soares, J.; et al. Systematic identification of genomic markers of drug sensitivity in cancer cells. Nature 2012, 483, 570-575, doi:10.1038/nature11005], which were made available through CancerRxGene (https://www.cancerrxgene.org/, accessed on 25 May 2023). In this study, we examined drug sensitivity using various measurements, including IC50, ln(IC50), EC50, and ln(EC50). Further details regarding the categorization of drug sensitivity and resistance can be found in our previous work [Ye, Q.; Mohamed, R.; Dukhlallah, D.; Gencheva, M.; Hu, G.; Pearce, M. C.; Kolluri, S. K.; Marsh, C. B.; Eubank, T. D.; Ivanov, A. V.; et al. Molecular Analysis of ZNF71 KRAB in Non-Small-Cell Lung Cancer. International journal of molecular sciences 2021, 22, doi:10.3390/ijms22073752, Ye, Q.; Singh, S.; Qian, P. R.; Guo, N. L. Immune-Omics Networks of CD27, PD1, and PDL1 in Non-Small Cell Lung Cancer. Cancers (Basel) 2021, 13, doi:10.3390/cancers13174296].

### Bioinformatic Tools

TarBase database v7.0 [Vlachos, I. S.; Paraskevopoulou, M. D.; Karagkouni, D.; Georgakilas, G.; Vergoulis, T.; Kanellos, I.; Anastasopoulos, I. L.; Maniou, S.; Karathanou, K.; Kalfakakou, D.; et al. DIANA-TarBase v7.0: indexing more than half a million experimentally supported miRNA:mRNA interactions. Nucleic Acids Res 2015, 43, D153-159, doi:10.1093/nar/gku1215] (https://dianalab.e-ce.uth.gr/html/universe/index.php?r=tarbase, accessed on 23 May 2023) and v8.0 [Karagkouni, D.; Paraskevopoulou, M. D.; Chatzopoulos, S.; Vlachos, I. S.; Tastsoglou, S.; Kanellos, I.; Papadimitriou, D.; Kavakiotis, I.; Maniou, S.; Skoufos, G.; et al. DIANA-TarBase v8: a decade-long collection of experimentally supported miRNA-gene interactions. Nucleic Acids Res 2018, 46, D239-D245, doi:10.1093/nar/gkx1141](https://dianalab.e-ce.uth.gr/html/diana/web/index.php?r=tarbasev8, accessed on 23 May 2023) was used to find experimentally validated microRNA-target interactions of our selected miRNAs.

ToppGene [Chen, J.; Bardes, E. E.; Aronow, B. J.; Jegga, A. G. ToppGene Suite for gene list enrichment analysis and candidate gene prioritization. Nucleic acids research 2009, 37, W305-311, doi:10.1093/nar/gkp427](https://toppgene.cchmc.org/, accessed on Jul. 6, 2023) is a comprehensive bioinformatics tool designed to facilitate gene list analysis and functional interpretation. It employs a variety of data mining and statistical techniques to uncover significant biological associations and gain insights into the functional relevance of gene sets. ToppGene provides a user-friendly platform for researchers to prioritize genes, unravel biological pathways, identify key biological functions, and discover potential disease associations. In this study, ToppGene was utilized to detect the functional enrichment of selected gene lists.

For drug repositioning analysis, we employed the bioinformatic tool Connectivity Map (CMap, https://clue.io/cmap, accessed on 23 May 2023). CMap utilizes gene expression profiles to determine connectivity scores based on drug-induced transcriptional profiles. The connectivity scores range from −1 to 1, with 1 indicating the highest degree of expression similarity. In this study, we considered a raw connectivity score higher than 0.9 with a p-value<0.05 as a significant result.

Cytoscape version 3.9.1 [Shannon, P.; Markiel, A.; Ozier, O.; Baliga, N. S.; Wang, J. T.; Ramage, D.; Amin, N.; Schwikowski, B.; Ideker, T. Cytoscape: a software environment for integrated models of biomolecular interaction networks. Genome research 2003, 13, 2498-2504, doi:10.1101/gr.1239303] was utilized to visualize the network results of miR-NAs and their target genes.

Statistical Analysis The Significance Analysis of Microarrays (SAM) method was used to identify miRNA markers displaying differential expression patterns in breast cancer samples. The following criteria were used to select statistically significant differentially expressed miRNAs: (1) an expression change threshold of >2 or <0.5 between normal and tumor tissues, or between blood samples from normal and cancer patients; (2) p-values less than 0.05 (unpaired or paired t-tests depended on the specific sample set being analyzed); and (3) a false discovery rate (FDR) below 0.05. Comparisons between two groups were assessed using two-sample t-tests. Survival analysis was conducted utilizing the Kaplan-Meier method implemented in the “survival” package (version 3.5.3). In the survival analysis, the log-rank test p-value was used to evaluate the difference in survival probabilities. Prognosis analysis was carried out employing the univariate Cox model. RStudio (version 2023.03.1 Build 446) with R version 4.2.1 was the primary statistical analysis tool throughout this study.

### Results

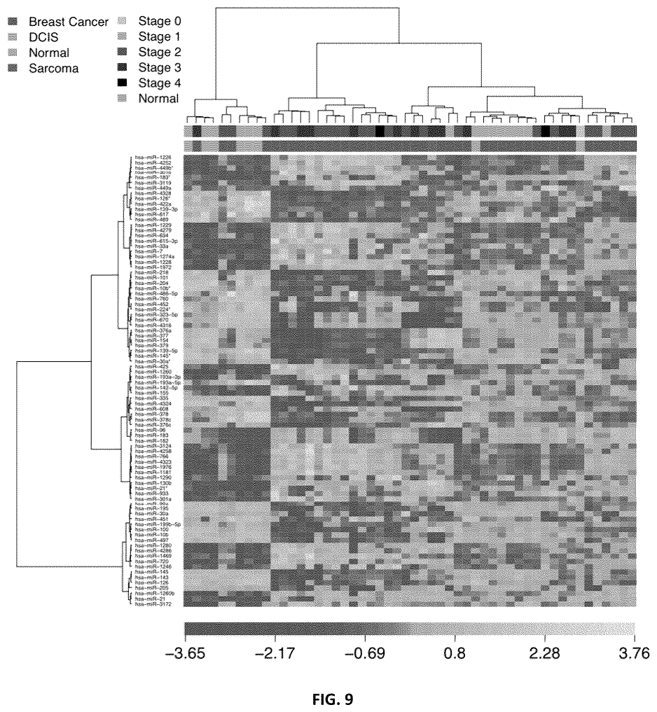

Differential MiRNA Expression in Breast Cancer Tissue and Blood. Using our patient cohort, we identified a collection of 86 miRNAs exhibiting significant differential expression when comparing tumor and normal breast tissue samples. To provide a visual representation of the expression patterns, FIG. 9 showcased a heatmap illustrating the clustering results of patient samples based on the expression profiles of these 86 miRNAs. In FIG. 9, four breast cancer samples were mistakenly classified into the normal breast tissue group, and one normal breast tissue sample was misclassified into the breast cancer group. The overall accuracy in the unsupervised classification was 90.4% using the expression profiles of 86 miRNAs. Here the denoted breast cancer in FIG. 9 referred to the cases with histology of ductal adenocarcinoma, invasive ductal carcinoma, invasive lobular carcinoma, metaplastic carcinoma, mucinous carcinoma, and predominantly lobular listed in Table 5.

Subsequently, it was investigated the prognostic significance of the identified 86 markers. Notably, 12 specific miRNAs shown in Table 6 displayed consistent diagnostic and prognostic relevance within both our patient cohort and TCGA breast cancer patients. In this analysis, miRNAs sharing the same prefix were analyzed together, i.e., the results were not miRNA isoform specific. The findings revealed that five miRNAs exhibited potential oncogenic characteristics (i.e., over-expression in tumors and survival hazard), while seven miRNAs demonstrated potential tumor suppressor properties in breast cancer (i.e., under-expression in tumors and survival protective).

TABLE 6
Potential oncogenic and potential tumor suppressive miRNAs with consistent diagnostic
and prognostic relevance within both our patient cohort and TCGA breast cancer cohort.

| | Fold | Fold | | | | | Potential |
|---|---|---|---|---|---|---|---|
| miRNA in our | Change | Change | miRNA in | Logrank | Hazard Ratio (95% | TCGA | Function |
| dataset | (unpaired) | (paired) | TCGA | p-value | confidence interval) | dataset | Category |
| hsa-miR-100 | 0.316 | 0.295 | hsa-miR-100 | 0.0195 | 0.766 [0.613, 0.9576] | GA, | tumor |
| | | | | | | n = 324 | suppressor |
| hsa-miR-101 | 0.474 | 0.278 | hsa-miR-101-1 | 0.0021 | 0.691 [0.546, 0.8741] | HiSeq, | tumor |
| | | | | | | n = 755 | suppressor |
| | | | hsa-miR-101-2 | 0.0278 | 0.805 [0.6626, 0.9769] | HiSeq, | |
| | | | | | | n = 755 | |
| hsa-miR-193a-3p | 2.070 | 2.244 | hsa-miR-193a | 0.0060 | 1.481 [1.1219, 1.9555] | HiSeq, | oncogene |
| | | | | | | n = 755 | |
| hsa-miR-204 | 0.241 | 0.148 | hsa-miR-204 | 0.0233 | 0.819 [0.6905, 0.9725] | HiSeq, | tumor |
| | | | | | | n = 755 | suppressor |
| hsa-miR-205 | 0.246 | 0.180 | hsa-miR-205 | 0.0335 | 0.871 [0.7667, 0.9904] | GA, | tumor |
| | | | | | | n = 324 | suppressor |
| hsa-miR-301b | 2.118 | 1.948 | hsa-miR-301b | 0.0100 | 1.410 [1.0837, 1.8342] | GA, | oncogene |
| | | | | | | n = 324 | |
| hsa-miR-30a | 0.406 | 0.324 | hsa-miR-30a | 0.0176 | 0.843 [0.7324, 0.9711] | HiSeq, | tumor |
| hsa-miR-30a* | 0.351 | 0.250 | | | | n = 755 | suppressor |
| hsa-miR-379 | 0.478 | 0.418 | hsa-miR-379 | 0.0480 | 0.797 [0.6369, 0.9981] | GA, | tumor |
| | | | | | | n = 324 | suppressor |
| hsa-miR-615-3p | 1.8673 | 2.531 | hsa-miR-615 | 0.0068 | 1.332 [1.0804, 1.6414] | GA, | oncogene |
| | | | | | | n = 324 | |
| hsa-miR-7 | 2.4657 | 3.994 | hsa-miR-7-2 | 0.0393 | 1.464 [1.0182, 2.1057] | GA, | oncogene |
| | | | | | | n = 324 | |
| | | | hsa-miR-7-3 | 0.0106 | 1.543 [1.1036, 2.1582] | GA, | |
| | | | | | | n = 324 | |
| hsa-miR-720 | 4.2969 | 5.964 | hsa-miR-720 | 0.0179 | 2.867 [1.189, 6.9117] | HiSeq, | oncogene |
| | | | | | | n = 755 | |
| hsa-miR-99a | 0.2919 | 0.242 | hsa-miR-99a | 0.0128 | 0.744 [0.5901, 0.939] | GA, | tumor |
| | | | | | | n = 324 | suppressor |

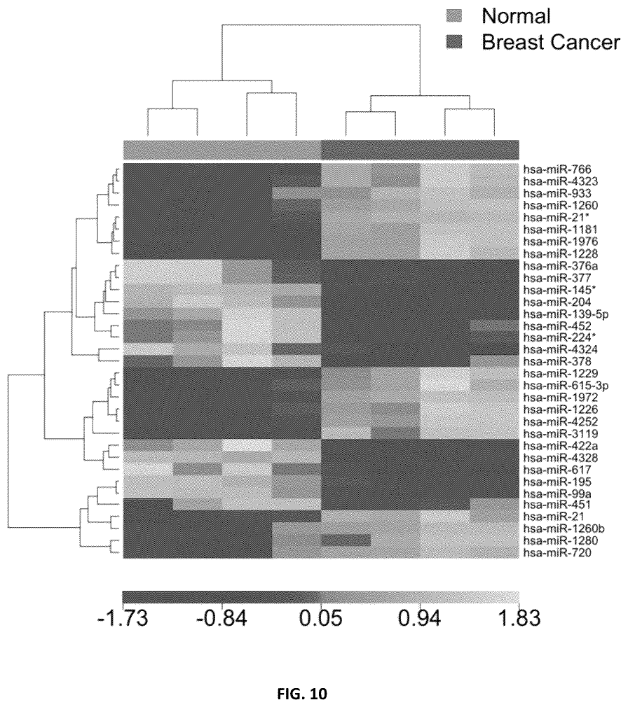

Out of the initial set of 86 markers, a total of 33 miRNAs exhibited statistically significant differential expression between matched tumors and normal breast tissue samples from the same patients. The clustering analysis of matched samples demonstrated a distinct separation with an overall classification accuracy of 100%, wherein all tumor tissues and all normal tissues were observed to cluster in different groups, as depicted in FIG. 10. This clustering pattern indicated a clear delineation between the gene expression profiles of breast cancer and normal samples.

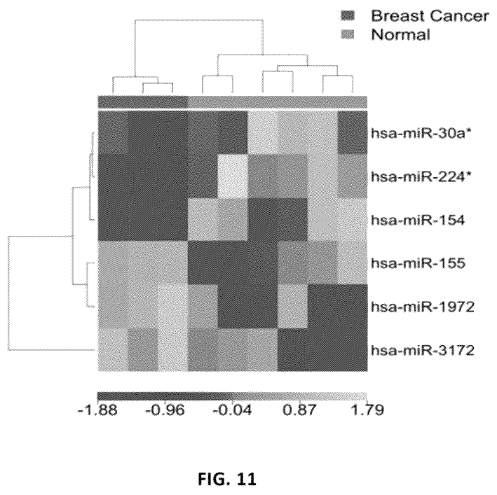

Among the analyzed miRNA markers, a subset of six markers exhibited concordant differential expression between breast cancer vs. normal in both tissues and blood samples. Utilizing these six markers, the breast cancer and normal blood samples could be effectively distinguished with an overall classification accuracy of 100%, as illustrated by the heatmap depicted in FIG. 11. This finding further underscored the potential of these markers in accurately discriminating between breast cancer and normal patient samples using minimally invasive blood tests.

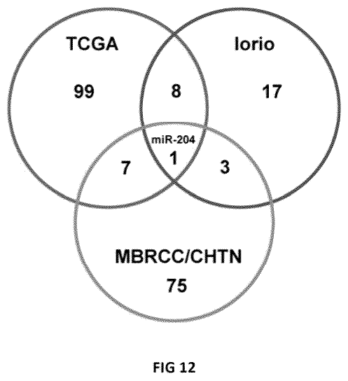

Next was examination of the miRNAs that displayed consistent differential expression patterns in our samples as well as in the cohort from Iorio et al. A total of four miRNAs (up-regulated miR-155, and down-regulated miR-204, miR-145, and miR-143) demonstrated statistically significant differential expression between tumors vs. normal breast tissues (FDR<0.05 in SAM, p<0.05; unpaired t-tests) in both the Iorio dataset (n=83) and our cohort (n=52). MiRNAs showing concordant expression patterns in breast cancer initiation and progression among our cohort, Iorio et al, and TCGA were depicted in FIG. 12. MiR-204 was a potential tumor suppressor based on the results from all three patient cohorts. These findings emphasized the robustness and reproducibility of the observed miRNA expression changes across different datasets and further supported the potential relevance of these miRNAs in breast cancer pathogenesis.

Association with Drug Sensitivity Among the 86 miRNAs identified in this study, a subset of 23 miRNAs was found in TarBase (v7 or v8) with experimentally validated target genes. These miRNAs collectively targeted a total of 4,117 genes, with published experimental evidence that was curated and stored in TarBase. To gain further insights into the clinical relevance of these miRNAs and their respective target genes, we investigated their associations with drug sensitivity in breast cancer treatment.

For drug sensitivity analysis, we classified the breast cancer cell lines as sensitive or resistant to each drug based on the measurements of IC50, ln(IC50), EC50, and ln(EC50), following the classification method described in our previous publications. Both mRNA and protein expression levels were considered in determining the association with drug sensitivity. A gene was deemed sensitive to a drug if it exhibited significantly higher expression (p<0.05; two-sample t-tests) in the sensitive cell line group. Conversely, a gene was classified as resistant to a drug if it demonstrated significantly higher expression (p<0.05; two-sample t-tests) in the resistant cell line group.

Additionally, we defined pan-sensitive genes as those exhibiting sensitivity or non-resistance to all 19 breast cancer drugs selected according to the NCCN guidelines (shown in Table 3), in the PRISM, GDSC1, and GDSC2 data. Conversely, pan-resistant genes were defined as those displaying resistance or non-sensitivity to all 19 breast cancer drugs in the aforementioned datasets. Table 7 presented the pan-sensitive and pan-resistant genes at both mRNA and protein levels, along with the pan-sensitive and pan-resistant miRNAs.

TABLE 7
Pan-sensitive and pan-resistant genes and miRNAs to 19 NCCN-recommended breast cancer (BRCA) drugs with concordant
expression at both mRNA and protein levels. mRNA level - italic font; protein level - normal font; both levels -
bold font. Genes harboring at least one non-silent mutation in any of the BRCA cell lines were highlighted
in underlined font. Genes associated with fusions only in BRCA cell lines were marked with parenthesis, while
genes exhibiting both fu-sions and non-silent mutations in BRCA cell lines were specifically highlighted in underlined
font and in parentheticals. The miRNAs that were also selected in Table 6 were highlighted with brackets.

| Drug | Pan-Sensitive Genes | Pan-Resistant Genes |
|---|---|---|
| 5-fluorouracil | GALM, HELLS, (RBM14), DUOX1, | KIF3A, KAT2B, MYADM, DDX3Y, |
| | RBM25, NOP56, DHX15, BAZ1B, | PTMS, EPHA7, EMC4, IRAK4, hsa- |
| | DGCR8, CD276, SNX13, PTPRF | miR-33a, hsa-miR-1260 |
| | PHIP, (TAOK1), SUZ12, PLEK, hsa- | |
| | miR-1246, hsa-miR-617 | |
| alpelisib | DUOX1, PTPRF, ZBED6, DGCR8, | KAT2B, EPHA7, BCL6, MTX3, IRGQ, |
| | TRABD, NOP56, RBM25, SUZ12, | TXNL1, IRAK4, GSK3A, APOD, |
| | GALM, WDR82, CDS2, hsa-miR-617, | KIF3A, MYADM, hsa-miR-1274a, hsa- |
| | hsa-miR-933 | miR-1280, hsa-miR-335, hsa-miR-376a, |
| | | hsa-miR-720 |
| capecitabine | | GSK3A, ZC3H4, |
| | | IRGQ, BCL6, MGST2, LZTS2 |
| carboplatin | WDR82 | |
| cisplatin | SNX13, GATB, PRDM2, | PTMS, PEG10, IRGQ, |
| | MFSD9, PHIP, | APOD, MTX3, |
| | TBRG1, CBX3, RPRD2, (RBM14), | (PTEN), STX3, ZNF24, LZTS2, |
| | PTPRF, EGR1, DHX15, PDE3A, | KIF3A, (FRYL), MYADM, |
| | ATG4D, (HERC6), DUOX1 | hsa-miR-301a, hsa-miR-497, hsa-miR-335, hsa-miR-1260 |
| cyclophosphamide | DHX15, RBM25, PHIP, DGCR8, | LZTS2, (PTEN), (FRYL), MYADM |
| | PRDM2, RPRD2, PTPRF, hsa-miR- | |
| | 1246 | |
| docetaxel | PDE3A, TBRG1, BAZ1B, DHX15, | GSK3A, ZC3H4, STX3, |
| | TBRG1, RBM25, RPRD2, hsa-miR- | (STMN3), IRAK4, APOD, |
| | 1246, hsa-miR-142-5p | KAT2B, EPHA7, PEG10 |
| doxorubicin | CDS2, (HERC6), NOP56, CD276, | KIF3A, PTMS, IRGQ, GSK3A, TTC26, |
| | TRABD, MFSD9, PHIP, HELLS, | hsa-miR-301a |
| | GATB, BAZ1B, NKTR, RBM25, | |
| | SUZ12, CBX3, (CHD9), (RBM14) | |
| epirubicin | NKTR, RBM25, TRABD, GATB, | (PTEN), ZNF24, LZTS2, EMC4, |
| | LSAMP, DHX15, PLEK | ZC3H4, MGST2, hsa-miR-1260, hsa- |
| | | miR-21, hsa-miR-301a |
| fulvestrant | BAZ1B, PRDM2, RBM25, CBX3, | MGST2, IRGQ, PSG6, APOD, IRAK4, |
| | SUZ12, (RBM14), HELLS, DHX15, | GSK3A, MYADM, hsa-miR-33a |
| | NKTR, DGCR8, (TAOK1), GATB, | |
| | TRABD, LSAMP, RPRD2, CD276, | |
| | hsa-miR-1976, hsa-miR-378, hsa-miR- | |
| | 766, hsa-miR-1246 | |
| gemcitabine | TBRG1, NOP56, MFSD9, RBM25, | APOD, PTMS, (MAVS), ZC3H4, hsa- |
| | BAZ1B | miR-301a |
| ixabepilone | DUOX1, TBRG1, ZBED6, (RBM14), | LZTS2, STX3, (PTEN), PEG10, KIF3A |
| | LSAMP, BAZ1B | |
| lapatinib | DUOX1, (HERC6), PLEK, PRDM2, | ZNF24, TTC26, KAT2B, PTMS, IRGQ, |
| | CD276, hsa-miR-933 | TXNL1, IRAK4, KIF3A, MYADM, |
| | | PSG6 |
| methotrexate | MFSD9, PHIP, RPRD2, CBX3, hsa- | MYADM, LZTS2, hsa-miR-497 |
| | miR-1246 | |
| neratinib | SNX13, ZBED6, PRDM2, hsa-miR-378, | TXNL1, MTX3, IRGQ, TTC26, EMC4, |
| | hsa-miR-10b | ZNF24, (STMN3), hsa-miR-21 |
| olaparib | PTPRF, (CHD9), GATB, PRDM2, | MTX3, (FRYL), BCL6, KAT2B, LZTS2, |
| | BAZ1B, CBX3, PDE3A, PHIP, ZBED6, | APOD, DDX3Y, MYADM |
| | MFSD9, TBRG1, CD276, SNX13 | |
| paclitaxel | GALM, DHX15, LSAMP, SNX13 | (MAVS), BCL6, ZC3H4, STX3, KAT2B, |
| | | MTX3, KIF3A, (STMN3), PEG10, |
| | | MYADM, PTMS, hsa-miR-21, hsa-miR- |
| | | 497 |
| tucatinib | CDS2, ATG4D, SNX13, PLEK, DUOX1, | MTX3, (PTEN), ZNF24, APOD, |
| | GATB, (CHD9), [hsa-miR-205], hsa- | GSK3A, hsa-miR-301a |
| | miR-378, hsa-miR-933, hsa-miR-376c | |
| vinorelbine | PRDM2, DGCR8, TRABD, EGR1, | MTX3, (MAVS), TTC26, EPHA7, hsa- |
| | BAZ1B, CD276, PTPRF, GATB, | miR-21, hsa-miR-199b-5p |
| | ATG4D, TBRG1, (TAOK1), WDR82, | |
| | SUZ12, (RBM14), NKTR, PDE3A, | |
| | RPRD2 | |

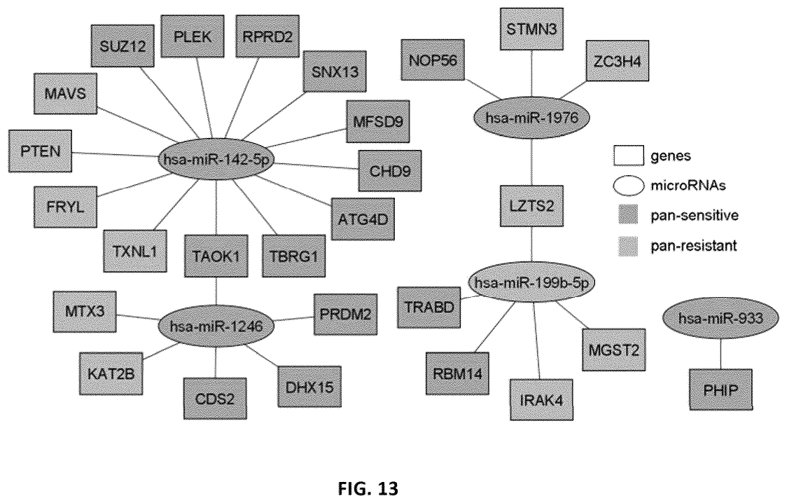

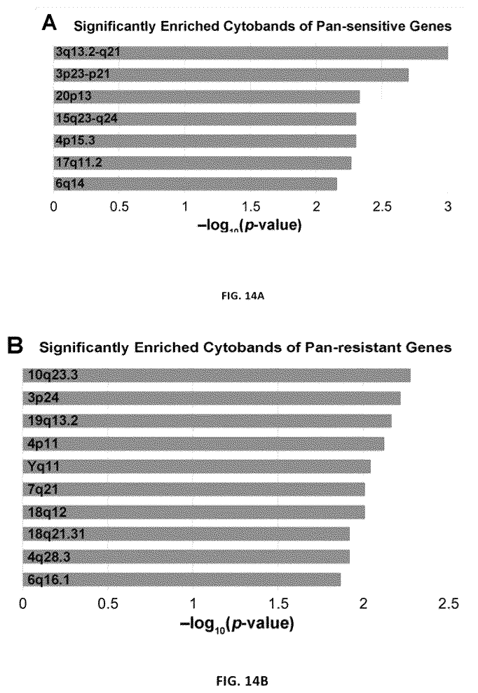

Among all the miRNAs and genes included in Table 3, we depicted miRNA-target genes and their association with pan-sensitivity/pan-resistance to 19 NCCN-recommended breast cancer drugs in FIG. 13. Furthermore, significantly enriched cytobands and gene families for pan-sensitive and pan-resistant genes were analyzed with ToppGene (FIG. 14). There were no significantly represented gene families for pan-sensitive genes. No significant pathways were enriched for either pan-sensitive or pan-resistant genes (Table 7). Through these comprehensive analyses, we aimed to uncover potential associations between the identified miRNAs, their target genes, and drug sensitivity, providing valuable insights into their roles in breast cancer biology and therapeutic response.

Discovery of Therapeutic Targets and Repositioning Drugs Since no significantly enriched pathways were found for pan-sensitive or pan-resistant genes, we further included patient survival genes and in-vitro proliferation genes to refine the gene list. To identify therapeutic targets and candidate compounds for treating breast cancer, we designed the mechanism of drug action to maintain the expression of non-proliferative survival-protective/pan-sensitive genes and suppress the survival-hazard/pan-resistant genes. Among the 4,117 genes targeted by our diagnostic miRNAs, we performed a refined selection to identify 28 up-regulated and 28 down-regulated gene lists for input into the CMap analysis. The 28 up-regulated genes met the following criteria: 1) exhibited a significant hazard ratio<1 (log-rank p<0.05) in survival analysis of TCGA-BRCA; 2) were classified as pan-sensitive genes for all 19 NCCN breast cancer drugs at both the mRNA and protein levels in CCLE BRCA cell lines; 3) did not display significant dependency scores (<−0.5) in more than 50% of the BRCA cell lines in both CRISPR-Cas9 and RNAi screening data. The 28 down-regulated genes met the following criteria: 1) demonstrated a significant hazard ratio>1 (log-rank p<0.05) in survival analysis of TCGA-BRCA; 2) were classified as pan-resistant genes for all 19 NCCN breast cancer drugs at both the mRNA and protein levels in CCLE BRCA cell lines.

It was then inputted the selected 28 up-regulated genes and the 28 down-regulated genes into CMap. From the CMap output, it was selected significant (raw connectivity score>0.9, p<0.05) pathways and compound sets as heuristic therapeutic targets. Then, the half-maximal inhibitory concentration (IC50) and half-maximal effective con-centration (EC50) of the significant compounds in the PRISM dataset was further examined, which led to the identification of 20 potential new or repurposed drugs with available IC50/EC50 measurements in the PRISM dataset. Our pipeline for repositioning drug discovery was shown in FIG. 7A. FIG. 7B displayed the boxplots of IC50/EC50 values of our discovered drugs in BRCA cell lines. Some of the drugs contained some outliers with extremely large values. The outliers were removed from the analysis and were not shown in the plot. These drugs belonged to 11 compound sets. In addition, three pathways were identified in shRNA knockdown experiments in breast adenocarcinoma MCF7 cells that matched our CMap input 56-gene expression signature (connectivity score>0.9, p<0.05), including knockdown of heat shock 70 (Hsp70) proteins (KD_HEAT_SHOCK_70KDA_PROTEINS:MCF7:TRT_SH.CGS) including HYOU1, HSPA5, HSPA9, and HSPA14 genes, SOD pathway (BIOCAR-TA_SODD_PATHWAY:MCF7:TRT_SH.CGS) including BAG4, BIRC3, CASP8, FADD, RIPK1, TNF, TNFRSF1A, TNFRSF1B, TRADD, and TRAF2, and knockdown of A-Kinase-Anchoring-Proteins (AKAPs; KD_A_KINASE_ANCHORING_PROTEINS:MCF7:TRT_SH.CGS) including AKAP9, AKAP13, and AKAP11.

Furthermore, we explored the genome-scale resistant and sensitive genes associated with our discovered drugs. Table 8 presented the selected sensitive and resistant genes for each drug, which had concordant expression at both mRNA and protein levels in the studied human breast cancer cell lines. Additionally, we identified gene fusions and mutations associated with the categorized sensitive and resistant cell lines to the corresponding drugs, highlighting the top fusion/mutation genes in each cell line. By integrating CMap analysis with our comprehensive multi-omics and drug sensitivity investigation, we unveiled potential novel therapeutic options and characterized patient responders/non-responders for these new breast cancer drugs (FIG. 5A).

TABLE 8
Sensitive and resistant genes with concordant mRNA and protein expression associated
with response to the new/repositioning drugs identified with CMap. Genes harboring
at least one non-silent mutation in any of the BRCA cell lines were highlighted
in bold font. Genes associated with fusions only in BRCA cell lines were marked
in underline font, while genes exhibiting both fu-sions and non-silent mutations
in BRCA cell lines were specifically highlighted in italic font.

| Drug | Sensitive Gene | Resistant Gene |
|---|---|---|
| Lestaurtinib | FHL1, FKBP7, LAMA4, LAMB3, | ARRB1 |
| | LAMC1, MMP14 | |
| BRD- | ACSL4, ASPH, CAV1, CAV2, EHD2, | ENPP1 |
| K12244279 | FMNL2, FSTL1, FYN, IFIT1, IFIT2, | |
| | LDHB, SAMD9, UPP1, WLS | |
| PD198306 | ICAM1, IFIT3 | |
| Pilocarpine | SDC2 | |
| TG101348 | VLDLR | ANXA8, APOL1, FBLN1, |
| | | ICAM1, MX2 |
| Tremorine | CRIP1, GPC4 | |
| U0126 | DKK3, FSD1, KYNU, LRP12 | |
| Y-27632 | C1R, CASP1, FOSL1, GSDMD | CLIC3, CPLX1, SYAP1 |

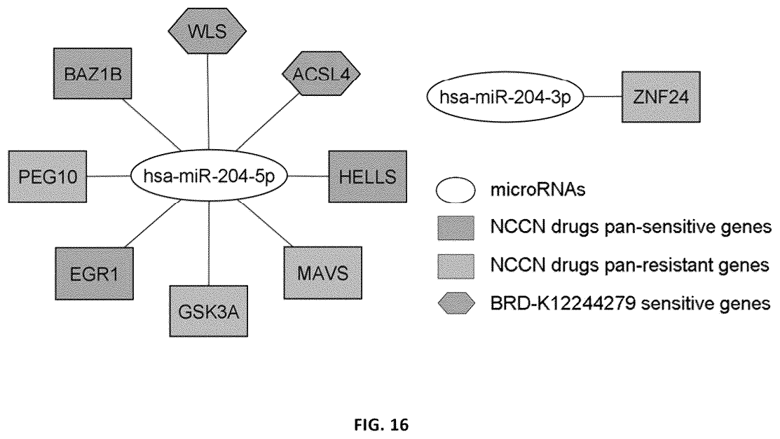

MiR-204 was identified as a potential tumor suppressor as under-expressed in tumors in our patient cohort and samples from Iorio et al. as well as patients with a poor prognosis in TCGA-BRCA (Table 6 and FIG. 12). MiR-204 was associated with drug sensitivity to carboplatin and tucatinib, and drug resistance to docetaxel, ixabepilone, paclitaxel, and vinorelbine. In addition, miR-204 was associated with resistance to RITA, a potential new drug option for treating breast cancer. Among these target genes, those that were pan-sensitive or pan-resistant to 19 NCCN-recommended breast cancer drugs (Table 7) were shown in FIG. 16. MiR-204 targeted genes (ACSL4 and WLS, Table 8) that were sensitive to BRD-K12244279 discovered for treating breast cancer in this study were also shown in FIG. 16.

Discussion This study identified 86 miRNAs differentially expressed between tumors and normal breast tissue samples in molecular classification. Four miRNA isoforms were confirmed in an external patient cohort as potential tissue-based breast cancer diagnostic biomarkers. Six miRNAs had concordant differential expression between breast cancer and normal samples in both tissue and blood, with miR-30a*, miR-224*, miR-154 downregulated and miR-155, miR-1972, miR-3172 upregulated in breast cancer. Twelve miRNAs had concordant expression patterns in our collected tumors vs. normal breast tissues and TCGA-BRCA patient survival hazard ratios, with five as potential oncogenes and seven as potential tumor suppressors. These results warranted further independent validation to substantiate the clinical utility of the identified miRNAs for breast cancer diagnosis using biopsies and/or liquid biopsies.

Among seven potential tumor-suppressing miRNAs identified in this study, high expression of miR-100 was associated with better outcomes in women with luminal A tumors treated with adjuvant endocrine therapy and was inversely linked to mRNA expression of PLK1, FOXA1, mTOR, and IGF1R [Petrelli, A.; Bellomo, S. E.; Sarotto, I.; Kubatzki, F.; Sgandurra, P.; Maggiorotto, F.; Di Virgilio, M. R.; Ponzone, R.; Geuna, E.; Galizia, D.; et al. MiR-100 is a predictor of endocrine responsiveness and prognosis in patients with operable luminal breast cancer. ESMO open 2020, 5, e000937, doi:10.1136/esmoopen-2020-000937]. By targeting FOXA1, miR-100 sup-pressed the migration, invasion, and proliferation of breast cancer cells [Xie, H.; Xiao, R.; He, Y.; He, L.; Xie, C.; Chen, J.; Hong, Y. MicroRNA-100 inhibits breast cancer cell proliferation, invasion and migration by targeting FOXA1. Oncology letters 2021, 22, 816, doi:10.3892/ol.2021.13077]. MiR-101-5p, a tumor suppressor, triggered apoptosis in HER2+ breast cancer and sensitized initially resistant cells to lapatinib and trastuzumab [Normann, L. S.; Haugen, M. H.; Aure, M. R.; Kristensen, V. N.; Molandsmo, G. M.; Sahlberg, K. K. miR-101-5p Acts as a Tumor Suppressor in HER2-Positive Breast Cancer Cells and Improves Targeted Therapy. Breast cancer (Dove Medical Press) 2022, 14, 25-39, doi:10.2147/bctt.s338404]. MiR-101 targeted Janus kinase 2 (JAK2) in inhibiting proliferation and promoting apoptosis of breast cancer cells [Wang, L.; Li, L.; Guo, R.; Li, X.; Lu, Y.; Guan, X.; Gitau, S. C.; Wang, L.; Xu, C.; Yang, B.; et al. miR-101 promotes breast cancer cell apoptosis by targeting Janus kinase 2. Cellular physiology and biochemistry: international journal of experimental cellular physiology, biochemistry, and pharmacology 2014, 34, 413-422, doi:10.1159/000363010]. MiR-204 was significantly under-expressed in breast cancer tumors in our patient cohort and samples from Iorio et al., as well as breast cancer patients with a poor survival outcome in TCGA-BRCA (FIG. 12). MiR-204-5p-regulated PI3K/Akt signaling inhibited tumor growth, metastasis, and immune cell reprogramming in breast cancer [Hong, B. S.; Ryu, H. S.; Kim, N.; Kim, J.; Lee, E.; Moon, H.; Kim, K. H.; Jin, M. S.; Kwon, N. H.; Kim, S.; et al. Tumor Suppressor miRNA-204-5p Regulates Growth, Metastasis, and Immune Microenvironment Remodeling in Breast Cancer. Cancer research 2019, 79, 1520-1534, doi:10.1158/0008-5472.can-18-0891]. By inhibiting miR-204-5p and enhancing RRM2 expression, DSCAM-AS1 promoted proliferation and suppressed apoptosis of breast cancer cells, indicating the therapeutic potential of DSCAM-AS1/miR-204-5p/RRM2 [Liang, W. H.; Li, N.; Yuan, Z. Q.; Qian, X. L.; Wang, Z. H. DSCAM-AS1 promotes tumor growth of breast cancer by reducing miR-204-5p and up-regulating RRM2. Molecular carcinogenesis 2019, 58, 461-473, doi:10.1002/mc.22941]. MiR-204-5p targets pan-sensitive genes (HELLS and BAZ1B), and multiple pan-resistant genes, including MAVS, GSK3A, and PEG10 identified in this study. MiR-204-3p targets a pan-resistant gene ZNF24 (FIG. 16). MiR-205 is a recognized tumor suppressor in breast cancer, with decreasing expression in breast cancer initiation, progression, metastasis, increased sternness [Zhang, L.; Liu, L.; Xu, X.; He, X.; Wang, G.; Fan, C.; Zheng, Q.; Li, F. miR-205/RunX2 axis negatively regulates CD44(+)/CD24(−) breast cancer stem cell activity. American journal of cancer research 2020, 10, 1871-1887], and more aggressive molecular subtypes [Plantamura, I.; Cataldo, A.; Cosentino, G.; Iorio, M. V. miR-205 in Breast Cancer: State of the Art. International journal of molecular sciences 2020, 22, doi:10.3390/ijms22010027]. MiR-205 was also identified as pan-sensitive to 19 NCCN-recommended breast cancer drugs in this study. MiR-30 family attenuated breast cancer invasion and bone metastasis as a promising therapeutic target for tri-ple-negative breast cancer [Croset, M.; Pantano, F.; Kan, C. W. S.; Bonnelye, E.; Descotes, F.; Alix-Panabieres, C.; Lecellier, C. H.; Bachelier, R.; Allioli, N.; Hong, S. S.; et al. miRNA-30 Family Members Inhibit Breast Cancer Invasion, Osteomimicry, and Bone Destruction by Directly Targeting Multiple Bone Metastasis-Associated Genes. Cancer research 2018, 78, 5259-5273, doi:10.1158/0008-5472.can-17-3058]. In this study, miR-30a* was under-expressed in both breast cancer tumors and blood samples compared with the corresponding normal controls, indicating its potential as a diagnostic biomarker. MiR-379 potently suppressed breast cancer formation and growth, partly through the regulation of COX2 [O'Brien, K. P.; Khan, S.; Gilligan, K. E.; Zafar, H.; Lalor, P.; Glynn, C.; O'Flatharta, C.; Ingoldsby, H.; Dockery, P.; De Bhulbh, A.; et al. Employing mesenchymal stem cells to support tumor-targeted delivery of extracellular vesicle (EV)-encapsulated microRNA-379. Oncogene 2018, 37, 2137-2149, doi:10.1038/s41388-017-0116-9] and inhibition of proliferation [Khan, S.; Brougham, C. L.; Ryan, J.; Sahrudin, A.; O'Neill, G.; Wall, D.; Curran, C.; Newell, J.; Kerin, M. J.; Dwyer, R. M. miR-379 regulates cyclin B1 expression and is decreased in breast cancer. PloS one 2013, 8, e68753, doi:10.1371/journal.pone.0068753]. MiR-379 and miR-204, both identified in this study as potential oncosuppressors, are key regulators of TGF-β-induced IL-11 production, important in breast cancer bone metastasis [Pollari, S.; Leivonen, S. K.; Perälä, M.; Fey, V.; Käkönen, S. M.; Kallioniemi, O. Identification of microRNAs inhibiting TGF-β-induced IL-11 production in bone metastatic breast cancer cells. PloS one 2012, 7, e37361, doi:10.1371/journal.pone.0037361]. MiR-99a inhibited breast cancer tumor-igenesis and progression by targeting the mTOR/p-4E-BP1/p-S6K1 pathway [Hu, Y.; Zhu, Q.; Tang, L. MiR-99a antitumor activity in human breast cancer cells through targeting of mTOR expression. PloS one 2014, 9, e92099, doi:10.1371/journal.pone.0092099] and the cell-cycle pathway through downregulating CDC25A [Qin, H.; Liu, W. MicroRNA-99a-5p suppresses breast cancer progression and cell-cycle pathway through downregulating CDC25A. Journal of cellular physiology 2019, 234, 3526-3537, doi:10.1002/jcp.26906]. MiR-99a-5p sensitized breast cancer cells to doxorubicin by regulating the COX-2/ABCG2 axis [Garrido-Cano, I.; Adam-Artigues, A.; Lameirinhas, A.; Blandez, J. F.; Candela-Noguera, V.; Rojo, F.; Zazo, S.; Madoz-Gúrpide, J.; Lluch, A.; Bermejo, B.; et al. miR-99a-5p modulates doxorubicin resistance via the COX-2/ABCG2 axis in triple-negative breast cancer: from the discovery to in vivo studies. Cancer communications (London, England) 2022, 42, 1412-1416, doi:10.1002/cac2.12352]. Our identified seven miRNAs are supported by the literature as BRCA tumor suppressors.

For the five identified potential oncomiRs, miR-193a-3p promoted tumor progression by targeting GRB7, ERK1/2, and FOXM1 signaling pathways [Tang, Y.; Yang, S.; Wang, M.; Liu, D.; Liu, Y.; Zhang, Y.; Zhang, Q. Epigenetically altered miR-193a-3p promotes HER2 positive breast cancer aggressiveness by targeting GRB7. International journal of molecular medicine 2019, 43, 2352-2360, doi:10.3892/ijmm.2019.4167] and PTP1B [Yu, M.; Liu, Z.; Liu, Y.; Zhou, X.; Sun, F.; Liu, Y.; Li, L.; Hua, S.; Zhao, Y.; Gao, H.; et al. PTP1B markedly promotes breast cancer progression and is regulated by miR-193a-3p. The FEBS journal 2019, 286, 1136-1153, doi:10.1111/febs.14724] in HER2-positive breast cancer cells. MiR-301b promoted cell proliferation by regulating PRKD3 in ER-mutant breast cancer, accounting for up to 30% of metastatic ER-positive breast cancer [Arnesen, S.; Polaski, J. T.; Blanchard, Z.; Osborne, K. S.; Welm, A. L.; O'Connell, R. M.; Gertz, J. Estrogen receptor alpha mutations regulate gene expression and cell growth in breast cancer through microRNAs. NAR cancer 2023, 5, zcad027, doi:10.1093/narcan/zcad027]. MiR-301b exerted tumor-promoting effects through co-regulation with its target gene NR3C2 in breast cancer MCF7 and BCAP-37 cells [Peng, Y.; Xi, X.; Li, J.; Ni, J.; Yang, H.; Wen, C.; Wen, M. miR-301b and NR3C2 co-regulate cells malignant properties and have the potential to be independent prognostic factors in breast cancer. Journal of biochemical and molecular toxicology 2021, 35, e22650, doi:10.1002/jbt.22650]. A miRNA in the same family, miR-301a, was identified as pan-resistant to 19 NCCN-recommended breast cancer drugs in this study. MiR-615-3p contributed to epithelial-to-mesenchymal transition and metastasis in breast cancer by regulating a negative feedback loop in-volving the PICK1/TGFBRI axis [Lei, B.; Wang, D.; Zhang, M.; Deng, Y.; Jiang, H.; Li, Y. miR-615-3p promotes the epithelial-mesenchymal transition and metastasis of breast cancer by targeting PICK1/TGFBRI axis. Journal of experimental & clinical cancer research: CR 2020, 39, 71, doi:10.1186/s13046-020-01571-]. Despite the association between miR-7 expression and patient poor survival, miR-7 inhibited breast cancer spreading and tumor-associated angiogenesis in metastatic breast cancer [Cui, Y. X.; Bradbury, R.; Flamini, V.; Wu, B.; Jordan, N.; Jiang, W. G. MicroRNA-7 suppresses the homing and migration potential of human endothelial cells to highly metastatic human breast cancer cells. British journal of cancer 2017, 117, 89-101, doi:10.1038/bjc.2017.156]. By modifying KLF4, miR-7 prevented breast cancer stem-like cells from metastasizing to the brain [Okuda, H.; Xing, F.; Pandey, P. R.; Sharma, S.; Watabe, M.; Pai, S. K.; Mo, Y. Y.; Iiizumi-Gairani, M.; Hirota, S.; Liu, Y.; et al. miR-7 suppresses brain metastasis of breast cancer stem-like cells by modulating KLF4. Cancer research 2013, 73, 1434-1444, doi:10.1158/0008-5472.can-12-2037]. Via activating the ERK signaling pathway, ADAM8 induced miR-720 expression, which in turn promoted the aggressive phenotype of triple-negative breast cancer cells [Das, S. G.; Romagnoli, M.; Mineva, N. D.; Barillé-Nion, S.; Jézéquel, P.; Campone, M.; Sonenshein, G. E. miR-720 is a downstream target of an ADAM8-induced ERK signaling cascade that promotes the migratory and invasive phenotype of triple-negative breast cancer cells. Breast cancer research: BCR 2016, 18, 40, doi:10.1186/s13058-016-0699-z.]. Overall, the literature supports our identified breast cancer oncomiRs, except for miR-7.

Upon the validation of our identified 86 miRNAs in multiple public patient cohorts, their experimentally validated target genes were retrieved with TarBase. Further analysis of these target genes pinpointed pan-sensitive and pan-resistant genes to 19 NCCN-recommended drugs for treating breast cancer. In the integrative analysis of the public in-vitro proliferation screening assay data of CCLE and TCGA-BRCA patient survival, a 56-gene expression signature was constructed to discover new and repositioning drugs for improving breast cancer treatment and survival outcomes using CMap. Through the CMap analysis, compounds that can inhibit the input down-regulated genes and maintain the expression of the input up-regulated genes in breast cancer cells were identified. The significant pathways and compound sets (raw connectivity score>0.9, p<0.05) were considered valid hypotheses worthy of further investigation. From these significant results, we further examined the compounds with average IC50/EC50 measurements less than 7 μM in human breast cancer cell lines as potential drugs for breast cancer treatment and were discussed as follows.

PI3K inhibitor SAR245409 combined with MEK inhibitor AS703026 (pimasertib) synergistically magnified anti-proliferative effects in TNBC [Lee, J.; Galloway, R.; Grandjean, G.; Jacob, J.; Humphries, J.; Bartholomeusz, C.; Goodstal, S.; Lim, B.; Bartholomeusz, G.; Ueno, N. T.; et al. Comprehensive Two- and Three-Dimensional RNAi Screening Identifies PI3K Inhibition as a Complement to MEK Inhibitor AS703026 for Combination Treatment of Triple-Negative Breast Cancer. Journal of Cancer 2015, 6, 1306-1319, doi:10.7150/jca.13266]. Lestaurtinib, a tyrosine kinase inhibitor, enhanced the in-vitro drug effects of the PARP1 inhibitor AG14361 in breast cancer treatment, partly by suppressing NF-κB signaling [Vazquez-Ortiz, G.; Chisholm, C.; Xu, X.; Lahusen, T. J.; Li, C.; Sakamuru, S.; Huang, R.; Thomas, C. J.; Xia, M.; Deng, C. Drug repurposing screen identifies lestaurtinib amplifies the ability of the poly (ADP-ribose) polymerase 1 inhibitor AG14361 to kill breast cancer associated gene-1 mutant and wild type breast cancer cells. Breast cancer research: BCR 2014, 16, R67, doi:10.1186/bcr3682]. Midostaurin is an FDA-approved multi-targeted protein kinase inhibitor for the treatment of both solid and non-solid tumors [Roskoski, R., Jr. Properties of FDA-approved small molecule protein kinase inhibitors: A 2022 update. Pharmacological research 2022, 175, 106037, doi:10.1016/j.phrs.2021.106037]. Midostaurin preferentially suppressed the proliferation of TNBC cells among breast cancer cell lines by inhibiting the Aurora kinase family [Kawai, M.; Nakashima, A.; Kamada, S.; Kikkawa, U. Midostaurin preferentially attenuates proliferation of triple-negative breast cancer cell lines through inhibition of Aurora kinase family. Journal of biomedical science 2015, 22, 48, doi:10.1186/s12929-015-0150-2]. Nilotinib (Tasigna) is an approved chronic myelogenous leukemia drug. Nilotinib can reduce doxorubicin-induced cardiac impairment [Huang, K. M.; Zavorka Thomas, M.; Magdy, T.; Eisenmann, E. D.; Uddin, M. E.; DiGiacomo, D. F.; Pan, A.; Keiser, M.; Otter, M.; Xia, S. H.; et al. Targeting OCT3 attenuates doxorubicin-induced cardiac injury. Proceedings of the National Academy of Sciences of the United States of America 2021, 118, doi:10.1073/pnas.2020168118]. Breast cancer cells resistant to tamoxifen therapy were resensitized by a combination of sorafenib and nilotinib via the estrogen receptor [Pedersen, A. M.; Thrane, S.; Lykkesfeldt, A. E.; Yde, C. W. Sorafenib and nilotinib resensitize tamoxifen resistant breast cancer cells to tamoxifen treatment via estrogen receptor α. International journal of oncology 2014, 45, 2167-2175, doi:10.3892/ijo.2014.2619]. MEK inhibitor CI-1040 (PD184352) exhibited notable antitumor activity in preclinical models, specifically against pancreatic, colon, and breast cancers, and was well tolerated in Phase I clinical trials [Allen, L. F.; Sebolt-Leopold, J.; Meyer, M. B. CI-1040 (PD184352), a targeted signal transduction inhibitor of MEK (MAPKK). Semin Oncol 2003, 30, 105-116, doi:10.1053/j.seminoncol.2003.08.012] but had insufficient efficacy in Phase II clinical studies [Rinehart, J.; Adjei, A. A.; Lorusso, P. M.; Waterhouse, D.; Hecht, J. R.; Natale, R. B.; Hamid, O.; Varterasian, M.; Asbury, P.; Kaldjian, E. P.; et al. Multicenter phase II study of the oral MEK inhibitor, CI-1040, in patients with advanced non-small-cell lung, breast, colon, and pancreatic cancer. Journal of clinical oncology: official journal of the American Society of Clinical Oncology 2004, 22, 4456-4462, doi:10.1200/jco.2004.01.185]. A second-generation MEK inhibitor PD0325901 was also selected using machine learning methods as a drug for breast cancer treatment [Mehmood, A.; Nawab, S.; Jin, Y.; Hassan, H.; Kaushik, A. C.; Wei, D. Q. Ranking Breast Cancer Drugs and Biomarkers Identification Using Machine Learning and Pharmacogenomics. ACS pharmacology & translational science 2023, 6, 399-409, doi:10.1021/acsptsci.2c00212] and ampli-fied anti-proliferative and anti-clonogenic effects of gefitinib and AT7867 by activating apoptosis in TNBC cells [You, K. S.; Yi, Y. W.; Cho, J.; Seong, Y. S. Dual Inhibition of AKT and MEK Pathways Potentiates the Anti-Cancer Effect of Gefitinib in Triple-Negative Breast Cancer Cells. Cancers 2021, 13, doi:10.3390/cancers13061205]. PD0325901 has much improved pharmacologic and pharmaceutical properties compared with CI-1040 and has also entered clinical development. Combination therapy of MEK inhibitor PD98059 and anti-diabetic drug Rosiglitazone caused invasive and spreading cancer cells to transform into post-mitotic adipocytes, which inhibited the invasion of the primary tumor and the development of metastases [Ishay-Ronen, D.; Diepenbruck, M.; Kalathur, R. K. R.; Sugiyama, N.; Tiede, S.; Ivanek, R.; Bantug, G.; Morini, M. F.; Wang, J.; Hess, C.; et al. Gain Fat-Lose Metastasis: Converting Invasive Breast Cancer Cells into Adipocytes Inhibits Cancer Metastasis. Cancer cell 2019, 35, 17-32.e16, doi:10.1016/j.ccell.2018.12.002]. U0126 is a highly potent and selective inhibitor specifically targeting MAPK, MEK1, and MEK2 signaling pathways and plays a pivotal role in maintaining cellular homeostasis [You, Y.; Niu, Y.; Zhang, J.; Huang, S.; Ding, P.; Sun, F.; Wang, X. U0126: Not only a MAPK kinase inhibitor. Frontiers in pharmacology 2022, 13, 927083, doi:10.3389/fphar.2022.927083]. U0126 reduced hyperpolarized pyruvate to lactate conversion, a non-imaging method for detecting tumors and treatment response, in breast cancer cells [Lodi, A.; Woods, S. M.; Ronen, S. M. Treatment with the MEK inhibitor U0126 induces decreased hyperpolarized pyruvate to lactate conversion in breast, but not prostate, cancer cells. NMR in biomedicine 2013, 26, 299-306, doi:10.1002/nbm.2848]. U0126 can reduce breast cancer cell content in the S phase, suggesting anti-proliferative effects by blocking the cell cycle [Zhao, L. Y.; Huang, C.; Li, Z. F.; Liu, L.; Ni, L.; Song, T. S. STAT1/2 is involved in the inhibition of cell growth induced by U0126 in HeLa cells. Cellular and molecular biology (Noisy-le-Grand, France) 2009, 55 Suppl, O11168-1174]. ERK phosphorylation in T47D breast cancer cells exhibited resistance to MEK inhibition by U0126, PD98059, and PD198306 [Aksamitiene, E.; Kholodenko, B. N.; Kolch, W.; Hoek, J. B.; Kiyatkin, A. PI3K/Akt-sensitive MEK-independent compensatory circuit of ERK activation in ER-positive PI3K-mutant T47D breast cancer cells. Cellular signalling 2010, 22, 1369-1378, doi:10.1016/j.cellsig.2010.05.006]. PD198306 has not been reported as a breast cancer drug that can prolong patient survival outcomes.

RITA small-molecule anticancer drug specifically targeting p53 [Doggrell, S. A. RITA—a small-molecule anticancer drug that targets p53. Expert opinion on investigational drugs 2005, 14, 739-742, doi:10.1517/13543784.14.6.739] has been extensively studied in breast cancer [Kaur, R. P.; Vasudeva, K.; Kumar, R.; Munshi, A. Role of p53 Gene in Breast Cancer: Focus on Mutation Spectrum and Therapeutic Strategies. Current pharmaceutical design 2018, 24, 3566-3575, doi:10.2174/1381612824666180926095709]. MEK inhibitor Selumetinib demonstrates clinical benefits in treating pediatric neurofibromatosis type I in phase II clinical trials [Gross, A. M.; Wolters, P. L.; Dombi, E.; Baldwin, A.; Whitcomb, P.; Fisher, M. J.; Weiss, B.; Kim, A.; Bornhorst, M.; Shah, A. C.; et al. Selumetinib in Children with Inoperable Plexiform Neurofibromas. New England Journal of Medicine 2020, 382, 1430-1442, doi:10.1056/NEJMoa1912735]. Selumetinib inhibits cell proliferation/migration, induces apoptosis/G1 arrest [Zhou, Y.; Lin, S.; Tseng, K. F.; Han, K.; Wang, Y.; Gan, Z. H.; Min, D. L.; Hu, H. Y. Selumetinib suppresses cell proliferation, migration and trigger apoptosis, G1 arrest in triple-negative breast cancer cells. BMC cancer 2016, 16, 818, doi:10.1186/s12885-016-2773-4], and prevents lung metastasis [Bartholomeusz, C.; Xie, X.; Pitner, M. K.; Kondo, K.; Dadbin, A.; Lee, J.; Saso, H.; Smith, P. D.; Dalby, K. N.; Ueno, N. T. MEK Inhibitor Selumetinib (AZD6244; ARRY-142886) Prevents Lung Metastasis in a Triple-Negative Breast Cancer Xenograft Model. Molecular cancer therapeutics 2015, 14, 2773-2781, doi:10.1158/1535-7163.mct-15-0243] in TNBC. Serdemetan (JNJ-26854165) is a small-molecule antagonist of MDM2, exhibiting antiproliferative activity in a range of tumor cell lines characterized by wild-type p53 [Chargari, C.; Leteur, C.; Angevin, E.; Bashir, T.; Schoentjes, B.; Arts, J.; Janicot, M.; Bourhis, J.; Deutsch, E. Preclinical assessment of JNJ-26854165 (Serdemetan), a novel tryptamine compound with radiosensitizing activity in vitro and in tumor xenografts. Cancer letters 2011, 312, 209-218, doi:10.1016/j.canlet.2011.08.011]. In a Phase I clinical study, Serdemetan was well tolerated by patients with advanced solid tumors with exposure-related QTc liability, and partial response was observed in a breast cancer patient [Tabernero, J.; Dirix, L.; Schoffski, P.; Cervantes, A.; Lopez-Martin, J. A.; Capdevila, J.; van Beijsterveldt, L.; Platero, S.; Hall, B.; Yuan, Z.; et al. A phase I first-in-human pharmacokinetic and pharmacodynamic study of serdemetan in patients with advanced solid tumors. Clinical cancer research: an official journal of the American Association for Cancer Research 2011, 17, 6313-6321, doi:10.1158/1078-0432.ccr-11-1101]. Sunitinib is an FDA-approved oral inhibitor of multi-target receptor tyrosine kinases and has demonstrated efficacy in the treatment of renal cell carcinoma and imatinib-resistant gastro-intestinal stromal tumor. Sunitinib achieved an overall response rate of 11% in treating metastatic breast cancer in a Phase II clinical trial [Burstein, H. J.; Elias, A. D.; Rugo, H. S.; Cobleigh, M. A.; Wolff, A. C.; Eisenberg, P. D.; Lehman, M.; Adams, B. J.; Bello, C. L.; DePrimo, S. E.; et al. Phase II Study of Sunitinib Malate, an Oral Multitargeted Tyrosine Kinase Inhibitor, in Patients With Metastatic Breast Cancer Previously Treated With an Anthracycline and a Taxane. Journal of Clinical Oncology 2008, 26, 1810-1816, doi:10.1200/jco.2007.14.5375]. Y-27632, a rho-kinase inhibitor, attenuates breast cancer cell migration, proliferation, and bone metastasis [Liu, S.; Goldstein, R. H.; Scepansky, E. M.; Rosenblatt, M. Inhibition of Rho-Associated Kinase Signaling Prevents Breast Cancer Metastasis to Human Bone. Cancer research 2009, 69, 8742-8751, doi:10.1158/0008-5472.can-09-1541]. MEK inhibitors BRD-K12244279 and PD98059 were identified as repurposing drug agents for treating vestibular schwannoma [Landry, A. P.; Wang, J. Z.; Suppiah, S.; Zadeh, G. Multiplatform molecular analysis of vestibular schwannoma reveals two robust subgroups with distinct microenvironment. Journal of neuro-oncology 2023, 161, 491-499, doi:10.1007/s11060-022-04221-2]. Pilocarpine is a cholinergic parasym-pathetic stimulant, generally used to treat xerostomia and oral cavities [Zur, E. Low-dose Pilocarpine Spray to Treat Xerostomia. International journal of pharmaceutical compounding 2020, 24, 104-108]. Tremorine was utilized to induce tremors and replicate symptoms resembling Parkinson's disease in animal models [Trautner, E. M.; Gershon, S. Use of tremorine for screening anti-parkinsonian drugs. Nature 1959, 183, 1462-1463, doi:10.1038/1831462a0]. Overall, 16 protein kinase inhibitors selected by our AI pipeline are promising targeted therapies for treating breast cancer, substantiating the effectiveness of our AI/ML methods. We discovered experimental agents, including MEK inhibitors PD19830 and BRD-K12244279, pilocarpine, and tremorine as potential new drug options for improving breast cancer survival outcomes, which were previously unknown.

Characterization of patient responders/non-responders is essential in designing clinical trials to test the efficacy of new and repositioning drugs. HCC1143 is a top in-vitro model to investigate TNBC and its transcriptional profiles are suitable to study the Interferon, IGF1, and MET signaling pathways [Grigoriadis, A.; Mackay, A.; Noel, E.; Wu, P. J.; Natrajan, R.; Frankum, J.; Reis-Filho, J. S.; Tutt, A. Molecular characterisation of cell line models for triple-negative breast cancers. BMC genomics 2012, 13, 619, doi:10.1186/1471-2164-13-619]. HCC1143 was sensitive to PD0325901, PD184352, PD198306, pilocarpine, RITA, serdemetan, tremorine, and Y-27632, and resistant to lestaurtinib, midostaurin, and nilotinib. HCC1806, another TNBC cell line with multi-drug resistance [Boichuk, S.; Galembikova, A.; Sitenkov, A.; Khusnutdinov, R.; Dunaev, P.; Valeeva, E.; Usolova, N. Establishment and characterization of a triple negative basal-like breast cancer cell line with multi-drug resistance. Oncology letters 2017, 14, 5039-5045, doi:10.3892/ol.2017.6795], was sensitive to midostaurin, nilotinib, PD0325901, PD198306, PD98059, pilocarpine, RITA, sorafenib, TG101348, tremorine, and Y-27632, and was resistant to AS703026, dasatinib, and lestaurtinib. HCC1937, a TNBC line derived from a 24-year-old woman with a family history of breast cancer and a germline mutation in BRCA1 [Chavez, K. J.; Garimella, S. V.; Lipkowitz, S. Triple negative breast cancer cell lines: one tool in the search for better treatment of triple negative breast cancer. Breast disease 2010, 32, 35-48, doi:10.3233/bd-2010-0307], was sensitive to pilocarpine, RITA, TG101348, and tremorine, and resistant to AS703026, dasatinib, midostaurin, nilotinib, PD0325901, PD198306, and PD98059. TNBC HCC38 cell line was sensitive to PD98059, pilocarpine, RITA, serdemetan, sorafenib, tremorine, and Y-27632, and resistant to dasatinib, les-taurtinib, midostaurin, nilotinib, PD0325901, PD184352, and PD198306. Luminal A MCF7 cells were sensitive to PD184352, PD198306, PD98059, RITA, TG101348, and U0126, and resistant to dasatinib, BRD-K12244279, and PD0325901. These results are useful for the clinical development of these drugs for treating breast cancer, especially TNBC.

Top occurring gene fusions and non-silent mutations in the above cell lines were pinpointed. The top fusion genes in HCC1143 were PPP2R5A and ZNRD1ASP, both having 8 fusions, and the top mutated gene is MUC3A with 6 mutations. SMURF2 had 6 fusions and TTN had 10 non-silent mutations in HCC1806. MUC3A had 7 mutations in HCC1937. COL24A1 had 12 fusions, and MUC3A and MUC5AC both had 6 mutations in HCC38. In MCF7, the top fusion genes were ATXN7 and VMP1, both having 6 fusions, and the top mutated gene was MUC3A with 7 mutations. MUC3A was the top mutated gene in multiple BRCA cell lines and promoted the progression of colorectal cancer through the PI3K/Akt/mTOR pathway [Su, W.; Feng, B.; Hu, L.; Guo, X.; Yu, M. MUC3A promotes the progression of colorectal cancer through the PI3K/Akt/mTOR pathway. BMC Cancer 2022, 22, 602, doi:10.1186/s12885-022-09709-8]. RAD51C-ATXN7 fusion gene expression was associated with functional damage of DNA repair and its fusion tran-script generated a fusion protein in colorectal tumors [Kalvala, A.; Gao, L.; Aguila, B.; Dotts, K.; Rahman, M.; Nana-Sinkam, S. P.; Zhou, X.; Wang, Q. E.; Amann, J.; Otterson, G. A.; et al. Rad51C-ATXN7 fusion gene expression in colorectal tumors. Molecular cancer 2016, 15, 47, doi:10.1186/s12943-016-0527-1]. RAD51C-ATXN7 fusion was also present in MCF7. The multimodal information presented in this study is important for future research and clinical management of breast cancer. The AI pipeline presented in this study can also be applied to model other data types for new/repositioning drug discovery, such as DNA copy number variation, transcriptomic, and proteomics profiles in bulk tumors and single cells for other cancer types as we previously published [Ye, Q.; Falatovich, B.; Singh, S.; Ivanov, A. V.; Eubank, T. D.; Guo, N. L. A Multi-Omics Network of a Seven-Gene Prognostic Signature for Non-Small Cell Lung Cancer. International journal of molecular sciences 2021, 23, doi:10.3390/ijms23010219, Ye, Q.; Guo, N. L. Single B Cell Gene Co-Expression Networks Implicated in Prognosis, Proliferation, and Therapeutic Responses in Non-Small Cell Lung Cancer Bulk Tumors. Cancers 2022, 14, 3123, Ye, Q.; Hickey, J.; Summers, K.; Falatovich, B.; Gencheva, M.; Eubank, T. D.; Ivanov, A. V.; Guo, N. L. Multi-Omics Immune Interaction Networks in Lung Cancer Tumorigenesis, Proliferation, and Survival. International journal of molecular sciences 2022, 23, 14978, Ye, Q.; Singh, S.; Qian, P. R.; Guo, N. L. Immune-Omics Networks of CD27, PD1, and PDL1 in Non-Small Cell Lung Cancer. Cancers 2021, 13, 4296, doi:10.3390/cancers13174296]. In the future, we will also apply this pipeline to model other structural variants for the discovery of biomarkers and therapeutic targets.

This study has several limitations. First, our patient cohort has a small sample size. The identified tissue-based 86 miRNAs and six blood-based miRNAs can separate breast cancer and normal samples with an overall accuracy of 90.4% and 100%, respectively. Nevertheless, due to the small sample size, their clinical utility in breast cancer diagnosis needs to be substantiated with the following studies: (1) an independent external patient cohort with a larger sample size; (2) high overall accuracy, specificity, and sensitivity in the external validation with the classification model fixed in the training-validation process; and (3) prospective validation of clinical benefits showing improved patient outcomes by using the diagnostic miRNA tests. Fulfilling these tasks will be time-consuming and require a vast amount of resources. Second, the functions of the identified potential oncomiRs and potential tumor-suppressing miRNAs need to be investigated in future research. Following the mechanistic characterization, the development of miRNA-based drugs needs to overcome technical challenges in achieving the optimal delivery route, intracellular efficacy, tissue specificity, and in-vivo stability. Finally, the identified compounds as new or repositioning drugs for breast cancer treatment were based on bioinformatics analysis and in-vitro IC50/EC50 measurements in human breast cancer cell lines. Their efficacy in treating breast cancer needs to be tested in future animal studies and/or clinical trials before obtaining regulatory approvals for clinical applications.

Conclusions. This study identified a set of 86 miRNAs to distinguish breast cancer tumors from normal breast tissues. Six miRNAs had concordant expression in both tumors and breast cancer patient blood samples compared with the normal control samples, with implications for the development of minimally-invasive diagnostic tests using liquid biopsies. Twelve miRNAs had concordant expression patterns in breast cancer initiation and progression, with seven as potential tumor suppressors and five as potential oncomiRs. From experimentally validated target genes of these 86 miRNAs, pan-sensitive and pan-resistant genes with concordant mRNA and protein expression associated with in-vitro drug response to 19 NCCN-recommended breast cancer drugs were selected. These genes, combined with in-vitro proliferation assays and patient survival analysis, led to the discovery of MEK inhibitors PD19830 and BRD-K12244279, pilocarpine, and tremorine as potential new drug options for treating breast cancer. Multi-omics biomarkers for response to the discovered drugs were identified using CCLE breast cancer cell lines. The presented AI pipeline utilizing multi-omics data analysis for the discovery of biomarkers and drug development can be applied to many human cancers.

### Breast Cancer New Drug Therapies

A set of 26-gene mRNA expression profiles that was used to identify invasive ductal carcinomas from histologically normal tissue and benign tissue and to select those with a higher potential for future cancer development (ADC) in the breast associated with atypical ductal hyperplasia (AD The expression defined model achieved an overall accuracy of 94.05% (AUC−0.96) in classifying the invasive ductal carcinomas from histologically normal tissue and benign lesions (n−185). This gene signature classified cancer development in AD tissues with an overall accuracy of 100% (n=8). The mRNA expression patterns of these 26 genes were validated using RT-PCR analysis of independent tissue samples (n=77) and blood samples (n=48). The protein expression of PBX2 and RAD52 assessed with immunohistochemistry were prognostic of breast cancer survival outcomes. This signature provided significant prognostic stratification in The Cancer Genome Atlas breast cancer patients (n=1,100), as well as basal-like and luminal A subtypes, and was associated with distinct immune infiltration and activities. The mRNA and protein expression of the 26 genes was associated with sensitivity or resistance to 18 NCCN-recommended drugs for treating breast cancer. Eleven genes had significant proliferative potential in CRISPR-Cas9/RNAi screening. Based on this gene expression signature, the VEGFR inhibitor ZM-306416 was discovered as a new drug for treating breast cancer.

Recent advancement in gene expression-based prognosis of breast cancer has also contributed to improved breast cancer treatment and overall survival. Oncotype DX [Paik, S.; Shak, S.; Tang, G.; Kim, C.; Baker, J.; Cronin, M.; Baehner, F. L.; Walker, M. G.; Watson, D.; Park, T.; et al. A multigene assay to predict recurrence of tamoxifen-treated, node-negative breast cancer. N. Engl. J. Med. 2004, 351, 2817-2826] and MammaPrint [van de Vijver, M. J.; He, Y. D.; van 't Veer, L. J.; Dai, H.; Hart, A. A.; Voskuil, D. W.; Schreiber, G. J.; Peterse, J. L.; Roberts, C.; Marton, M. J.; et al. A gene-expression signature as a predictor of survival in breast cancer. N. Engl. J. Med. 2002, 347, 1999-2009, van 't Veer, L. J.; Dai, H.; van de Vijver, M. J.; He, Y. D.; Hart, A. A.; Mao, M.; Peterse, H. L.; van der Kooy, K.; Marton, M. J.; Witteveen, A. T.; et al. Gene expression profiling predicts clinical outcome of breast cancer. Nature 2002, 415, 530-536] are available for ER-positive early-stage patients. The cost of Oncotype DX is covered by major healthcare insurance companies and the Medicare/Medicaid program in the US. After Oncotype DX was made commercially available to clinics, it entered a prospective clinical trial that finalized the patient stratification schemes in 2018 [Sparano, J. A.; Gray, R. J.; Ravdin, P. M.; Makower, D. F.; Pritchard, K. I.; Albain, K. S.; Hayes, D. F.; Geyer, C. E., Jr.; Dees, E. C.; Goetz, M. P.; et al. Clinical and Genomic Risk to Guide the Use of Adjuvant Therapy for Breast Cancer. N. Engl. J. Med. 2019, 380, 2395-2405. https://doi.org/10.1056/NEJMoa1904819, Sparano, J. A.; Gray, R. J.; Makower, D. F.; Pritchard, K. I.; Albain, K. S.; Hayes, D. F.; Geyer, C. E., Jr.; Dees, E. C.; Goetz, M. P.; Olson, J. A., Jr.; et al. Adjuvant Chemotherapy Guided by a 21-Gene Expression Assay in Breast Cancer. N. Engl. J. Med. 2018, 379, 111-121. https://doi.org/10.1056/NEJMoa1804710]. Breast cancer subtypes based on gene expression profiling [Perou, C. M.; Jeffrey, S. S.; van de Rijn, M.; Rees, C. A.; Eisen, M. B.; Ross, D. T.; Pergamenschikov, A.; Williams, C. F.; Zhu, S. X.; Lee, J. C.; et al. Distinctive gene expression patterns in human mammary epithelial cells and breast cancers. Proc. Natl. Acad. Sci. USA 1999, 96, 9212-9217, Perou, C. M.; Sorlie, T.; Eisen, M. B.; van de Rijn, M.; Jeffrey, S. S.; Rees, C. A.; Pollack, J. R.; Ross, D. T.; Johnsen, H.; Akslen, L. A.; et al. Molecular portraits of human breast tumours. Nature 2000, 406, 747-752, Sorlie, T.; Perou, C. M.; Tibshirani, R.; Aas, T.; Geisler, S.; Johnsen, H.; Hastie, T.; Eisen, M. B.; van de Rijn, M.; Jeffrey, S. S.; et al. Gene expression patterns of breast carcinomas distinguish tumor subclasses with clinical implications. Proc. Natl. Acad. Sci. USA 2001, 98, 10869-10874], PAM50 (prediction of microarray using 50 classifier genes plus 5 reference genes) [Parker, J. S.; Mullins, M.; Cheang, M. C.; Leung, S.; Voduc, D.; Vickery, T.; Davies, S.; Fauron, C.; He, X.; Hu, Z.; et al. Supervised risk predictor of breast cancer based on intrinsic subtypes. J. Clin. Oncol. 2009, 27, 1160-1167. https://doi.org/10.1200/jco.2008.18.1370], the Nottingham Prognostic Index Plus (NPI+) [Winslow, S.; Leandersson, K.; Edsjö, A.; Larsson, C. Prognostic stromal gene signatures in breast cancer. Breast Cancer Res. 2015, 17, 23. https://doi.org/10.1186/s13058-015-0530-2], the Breast Cancer Index [Sgroi, D. C.; Sestak, I.; Cuzick, J.; Zhang, Y.; Schnabel, C. A.; Schroeder, B.; Erlander, M. G.; Dunbier, A.; Sidhu, K.; Lopez-Knowles, E.; et al. Prediction of late distant recurrence in patients with oestrogen-receptor-positive breast cancer: A prospective comparison of the breast-cancer index (BCI) assay, 21-gene recurrence score, and IHC4 in the TransATAC study population. Lancet Oncol. 2013, 14, 1067-1076. https://doi.org/10.1016/si470-2045(13)70387-5], The Rotterdam Signature [Wang, Y.; Klijn, J. G.; Zhang, Y.; Sieuwerts, A. M.; Look, M. P.; Yang, F.; Talantov, D.; Timmermans, M.; Meijer-van Gelder, M. E.; Yu, J.; et al. Gene-expression profiles to predict distant metastasis of lymph-node-negative primary breast cancer. Lancet 2005, 365, 671-679], and EndoPredict [Müller, B. M.; Keil, E.; Lehmann, A.; Winzer, K. J.; Richter-Ehrenstein, C.; Prinzler, J.; Bangemann, N.; Reles, A.; Stadie, S.; Schoenegg, W.; et al. The EndoPredict Gene-Expression Assay in Clinical Practice—Performance and Impact on Clinical Decisions. PLoS ONE 2013, 8, e68252. https://doi.org/10.1371/journal.pone.0068252] were also developed to aid treatment selection for invasive breast cancers. Other gene signatures for breast cancer prognosis were identified based on wound-healing response [Chang, H. Y.; Nuyten, D. S.; Sneddon, J. B.; Hastie, T.; Tibshirani, R.; Sorlie, T.; Dai, H.; He, Y. D.; van 't Veer, L. J.; Bartelink, H.; et al. Robustness, scalability, and integration of a wound-response gene expression signature in predicting breast cancer survival. Proc. Natl. Acad. Sci. USA 2005, 102, 3738-3743], immune response [Teschendorff, A. E.; Miremadi, A.; Pinder, S. E.; Ellis, I. O.; Caldas, C. An immune response gene expression module identifies a good prognosis subtype in estrogen receptor negative breast cancer. Genome Biol. 2007, 8, R157. https://doi.org/10.1186/gb-2007-8-8-r157], and stromal gene expression.

Among the breast cancer subtypes, triple-negative breast cancer (TNBC, and basal-like) does not express hormone receptors and, thus, has limited therapeutic options [Prat, A.; Pineda, E.; Adamo, B.; Galvin, P.; Fernández, A.; Gaba, L.; Diez, M.; Viladot, M.; Arance, A.; Munoz, M. Clinical implications of the intrinsic molecular subtypes of breast cancer. Breast 2015, 24 (Suppl. S2), S26-S35. https://doi.org/10.1016/j.breast.2015.07.008]. TNBC accounts for 10-20% of breast cancer cases. Due to the high number of tumor-infiltrating lymphocytes (TILs), TNBC is considered the most immunogenic breast cancer subtype [Hammerl, D.; Smid, M.; Timmermans, A. M.; Sleijfer, S.; Martens, J. W. M.; Debets, R. Breast cancer genomics and immuno-oncological markers to guide immune therapies. Semin. Cancer Biol. 2018, 52 Pt 2, 178-188. https://doi.org/10.1016/j.semcancer.2017.11.003]. Anti-PD-L1 (atezolizumab) combined with nab-paclitaxel suggested survival benefits in locally advanced or metastatic TNBC patients in a randomized phase III clinical trial [Schmid, P.; Rugo, H. S.; Adams, S.; Schneeweiss, A.; Barrios, C. H.; Iwata, H.; Diéras, V.; Henschel, V.; Molinero, L.; Chui, S. Y.; et al. Atezolizumab plus nab-paclitaxel as first-line treatment for unresectable, locally advanced or metastatic triple-negative breast cancer (IMpassion130): Updated efficacy results from a randomised, double-blind, placebo-controlled, phase 3 trial. Lancet Oncol. 2020, 21, 44-59. https://doi.org/10.1016/s1470-2045(19)30689-8]. Nevertheless, a significant portion of TNBC patients do not respond to immunotherapy. Tumor mutation burdens and PD-L1 expression are not associated with response to immune checkpoint inhibitors in breast cancer [Voorwerk, L.; Slagter, M.; Horlings, H. M.; Sikorska, K.; van de Vijver, K. K.; de Maaker, M.; Nederlof, I.; Kluin, R. J. C.; Warren, S.; Ong, S.; et al. Immune induction strategies in metastatic triple-negative breast cancer to enhance the sensitivity to PD-1 blockade: The TONIC trial. Nat. Med. 2019, 25, 920-928. https://doi.org/10.1038/s41591-019-0432-4, Schmid, P.; Cortes, J.; Pusztai, L.; McArthur, H.; Kummel, S.; Bergh, J.; Denkert, C.; Park, Y. H.; Hui, R.; Harbeck, N.; et al. Pembrolizumab for Early Triple-Negative Breast Cancer. N. Engl. J. Med. 2020, 382, 810-821. https://doi.org/10.1056/NEJMoa1910549]. Novel biomarkers and therapeutic options are needed to improve TNBC treatment outcomes.

Despite the novel discoveries and successful clinical applications of molecular biomarkers for breast cancer prognosis, there are currently no clinically applied multi-gene assays for early breast cancer detection. More than 80% of breast cancer cases are discovered when a lump in the breast is detected with the fingertips [Choi, L. Breast Cancer. 2022 September 2022. Availabe online: https://www.merckmanuals.com/home/women-s-health-issues/breast-disorders/breast-cancer (accessed on 17 Apr. 2023)]. Current breast cancer screening tools such as mammograms are controversial in terms of benefits versus harm for use in routine testing [Gotzsche, P. C.; Jorgensen, K. J. Screening for breast cancer with mammography. Cochrane Database Syst. Rev. 2013, 2013, Cd001877. https://doi.org/10.1002/14651858.CD001877.pub5, Nelson, H. D.; Tyne, K.; Naik, A.; Bougatsos, C.; Chan, B.; Nygren, P.; Humphrey, L. U. S. Preventive Services Task Force Evidence Syntheses, formerly Systematic Evidence Reviews. In Screening for Breast Cancer: Systematic Evidence Review Update for the US Preventive Services Task Force; Agency for Healthcare Research and Quality (US): Rockville, MD, USA, 2009]. The medium mammogram-detectable tumor size was reported as 7.5 mm [Shaevitch, D.; Taghipour, S.; Miller, A. B.; Montgomery, N.; Harvey, B. Tumor size distribution of invasive breast cancers and the sensitivity of screening methods in the Canadian National Breast Screening Study. J. Cancer Res. Ther. 2017, 13, 562-569. https://doi.org/10.4103/0973-1482.174539]. False results could arise due to technical issues or a tumor size smaller than that. Molecular assays for the intraoperative evaluation of sentinel nodes have been developed using cytokeratin 19 (CK19) mRNA amplification [Laia, B. V.; Marcos, M. B.; Refael, C. M.; Francisco, S. C.; Jose, T.; Blai, B. S. Molecular Diagnosis of Sentinel Lymph Nodes for Breast Cancer: One Step Ahead for Standardization. Diagn. Mol. Pathol. 2011, 20, 18-21. https://doi.org/10.1097/PDM.0b013e3181eb9b30] and mammaglobin (MG) and CK19 immunohistochemistry (IHC), both achieving high specificity and sensitivity. The molecular diagnosis of triple-negative breast cancer is primarily based on the IHC of the estrogen receptor (ER), progesterone receptor (PR), and human epidermal growth factor receptor 2 (HER2) [Penault-Llorca, F.; Viale, G. Pathological and molecular diagnosis of triple-negative breast cancer: A clinical perspective. Ann. Oncol. Off J. Eur. Soc. Med. Oncol. 2012, 23 (Suppl. S6), vi19-vi22. https://doi.org/10.1093/annonc/mds190]. EGFR/IiER2 assessment is used in breast molecular diagnosis and therapy [Milanezi, F.; Carvalho, S.; Schmitt, F. C. EGFR/IER2 in breast cancer: A biological approach for molecular diagnosis and therapy. Expert Rev. Mol. Diagn. 2008, 8, 417-434. https://doi.org/10.1586/14737159.8.4.417]. Specific translocations characterizing some special types of mammary carcinomas could potentially be used as diagnostic companion tests [Rakha, E. A.; Green, A. R. Molecular classification of breast cancer: What the pathologist needs to know. Pathology 2017, 49, 111-119. https://doi.org/10.1016/j.pathol.2016.10.012]. A balanced translocation between chromosomes 12 and 15 creating a new ETV6-NTRK3 fusion gene is a primary event in human secretory breast carcinoma, a rare form of breast cancer [Tognon, C.; Knezevich, S. R.; Huntsman, D.; Roskelley, C. D.; Melnyk, N.; Mathers, J. A.; Becker, L.; Carneiro, F.; MacPherson, N.; Horsman, D.; et al. Expression of the ETV6-NTRK3 gene fusion as a primary event in human secretory breast carcinoma. Cancer Cell 2002, 2, 367-376. https://doi.org/10.1016/si535-6108(02)00180-0]. Adenoid cystic carcinoma of the breast, a rare histological type of triple-negative breast cancer with indolent clinical behavior, is characterized by the MYB-NFIB fusion gene [Fusco, N.; Geyer, F. C.; De Filippo, M. R.; Martelotto, L. G.; Ng, C. K.; Piscuoglio, S.; Guerini-Rocco, E.; Schultheis, A. M.; Fuhrmann, L.; Wang, L.; et al. Genetic events in the progression of adenoid cystic carcinoma of the breast to high-grade triple-negative breast cancer. Mod. Pathol. 2016, 29, 1292-1305. https://doi.org/10.1038/modpathol.2016.134]. Approximately 5-10% of breast cancer incidents are deemed inheritable [32]. Genetic tests based on BRCA1 and BRCA2 are clinically available to estimate hereditary breast-ovarian cancer familial risks, which account for about 90% of inheritable cases. Other gene mutations were linked to a substantial minority of inheritable breast cancers, including TP53, PTEN, STK11, CHEK2, ATM, BRIP1, and PALB2 [Gage, M.; Wattendorf, D.; Henry, L. R. Translational advances regarding hereditary breast cancer syndromes. J. Surg. Oncol. 2012, 105, 444-451. https://doi.org/10.1002/jso.21856]. For the majority of the breast cancer patient population, there are currently no clinically applicable gene tests to detect a precancerous state in histologically normal tissue and benign lesions or to predict which premalignant lesions will develop into invasive breast cancer. Specifically, biomarkers found in biological fluids, blood in particular, are the most promising for the fast development of screening assays for early detection of breast cancer [Levenson, V. V. Biomarkers for early detection of breast cancer: What, when, and where?Biochim. et Biophys. Acta 2007, 1770, 847-856. https://doi.org/10.1016/j.bbagen.2007.01.017].

Emerging biomarkers for breast cancer diagnosis should provide additional information relevant to prognosis and the selection of therapy. We previously identified and validated a 28-gene prognostic signature in more than 2000 breast cancer patients in both early and late stages of any ER or lymph node status at the time of diagnosis [Ma, Y.; Qian, Y.; Wei, L.; Abraham, J.; Shi, X.; Castranova, V.; Harner, E. J.; Flynn, D. C.; Guo, L. Population-Based Molecular Prognosis of Breast Cancer by Transcriptional Profiling. Clin. Cancer Res. 2007, 13, 2014-2022, Rathnagiriswaran, S.; Wan, Y. W.; Abraham, J.; Castranova, V.; Qian, Y.; Guo, N. L. A population-based gene signature is predictive of breast cancer survival and chemoresponse. Int. J. Oncol. 2010, 36, 607-616]. It is also prognostic of the clinical outcome in multiple epithelial cancers, including ovarian cancer [Rathnagiriswaran and Wan, Y. W.; Qian, Y.; Rathnagiriswaran, S.; Castranova, V.; Guo, N. L. A breast cancer prognostic signature predicts clinical outcomes in multiple tumor types. Oncol. Rep. 2010, 24, 489-494]. With the evidence of its prognostic capacity established in our previous studies, this study sought to investigate (1) its potential clinical utility in breast cancer diagnosis in solid tissues and blood samples that we collected; (2 its ability to further refine prognosis within TNBC using The Cancer Genome Atlas (TCGA); (3) its association with immune infiltration and immune cell type activities in TCGA breast cancer patient tumors; (4) its proliferation potential in CRISPR-Cas9/RNAi screening in human breast cancer cell lines in the public Cancer Cell Line Encyclopedia (CCLE); (5) its association with 18 National Comprehensive Cancer Network (NCCN)-recommended drugs for treating breast cancer in the CCLE panel. Finally, based on this 28-gene expression signature, potential new drug options for treating breast cancer were identified using Connectivity Map (CMap) [Subramanian, A.; Narayan, R.; Corsello, S. M.; Peck, D. D.; Natoli, T. E.; Lu, X.; Gould, J.; Davis, J. F.; Tubelli, A. A.; Asiedu, J. K.; et al. A Next Generation Connectivity Map: L1000 Platform and the First 1,000,000 Profiles. Cell 2017, 171, 1437-1452.e1417. https://doi.org/10.1016/j.cell.2017.10.049, Lamb, J.; Crawford, E. D.; Peck, D.; Modell, J. W.; Blat, I. C.; Wrobel, M. J.; Lerner, J.; Brunet, J. P.; Subramanian, A.; Ross, K. N.; et al. The Connectivity Map: Using gene-expression signatures to connect small molecules, genes, and disease. Science 2006, 313, 1929-1935. https://doi.org/10.1126/science.1132939].

### Results

28-Gene Signature as a Diagnostic Assay for Breast Cancer We studied the diagnostic performance of the 28-gene signature in two independent patient cohorts. In both cohorts, the M5P algorithm was used to construct a classifier with the signature genes to identify invasive breast cancer from normal, benign, or premalignant tissues. The final prediction performance was computed from the leave-one-out cross-validation.

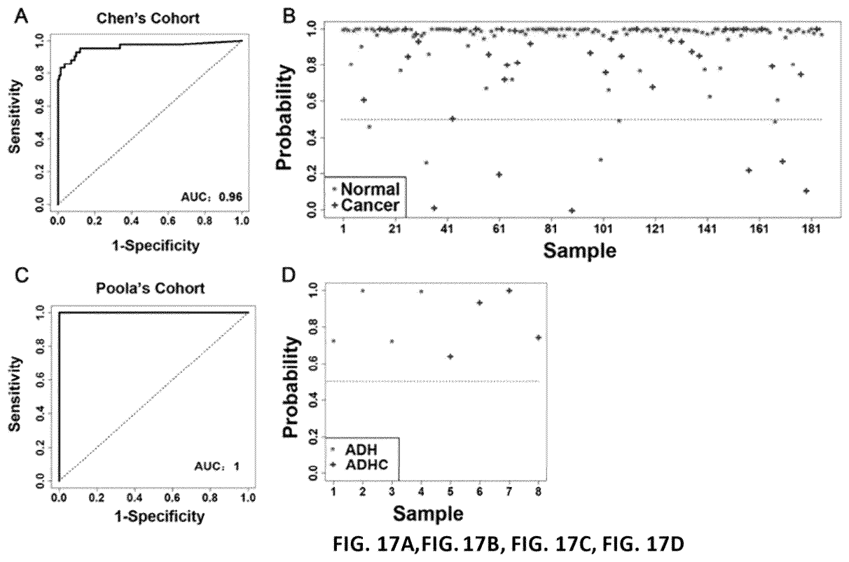

In the first cohort from Chen et al. [Chen, D. T.; Nasir, A.; Culhane, A.; Venkataramu, C.; Fulp, W.; Rubio, R.; Wang, T.; Agrawal, D.; McCarthy, S. M.; Gruidl, M.; et al. Proliferative genes dominate malignancy-risk gene signature in histologically-normal breast tissue 1. Breast Cancer Res. Treat. 2010, 119, 335-346] (n=185), the constructed classifier was highly precise in discriminating normal breast tissues from breast cancer tumors, with an area under the ROC curve (AUC) of 0.96 (FIG. 17A). Specifically, using the expression profile of the 26 available genes as predictors, the malignant scores estimated by the classifier correctly identified normal breast tissue samples from invasive ductal carcinoma samples 94% of the time (FIG. 17B). In the second cohort of patients with atypical ductal hyperplasia (ADH) from Poola et al. [40] (n=8), using the 25 available genes, the M5P classifier perfectly discriminated between patients with or without invasive breast cancer (AUC=1, accuracy=100%; FIGS. 17C and 17D). These results demonstrate that the 28-gene signature is a strong classifier of breast cancer tumors versus benign tissues, with potential implications in breast cancer diagnosis.

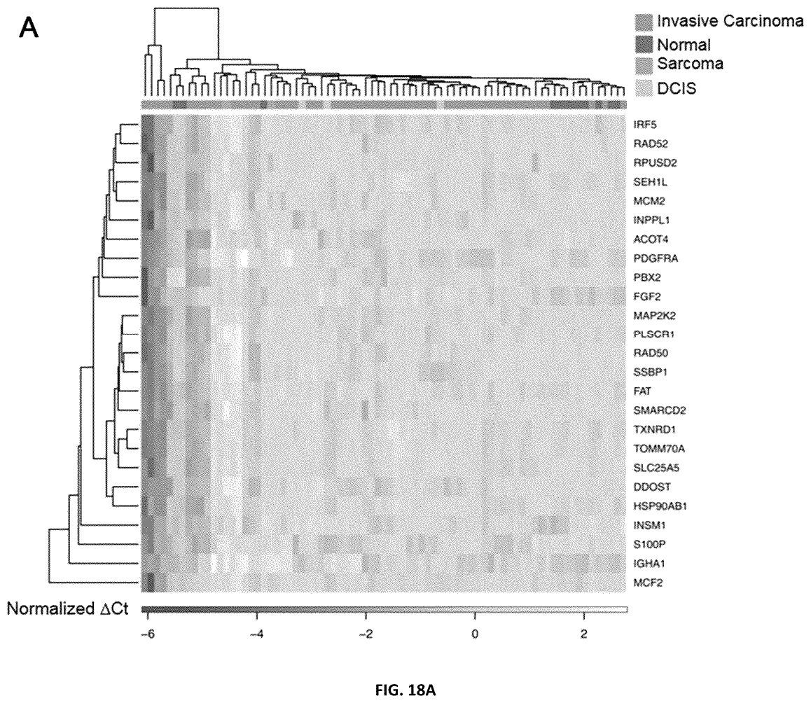

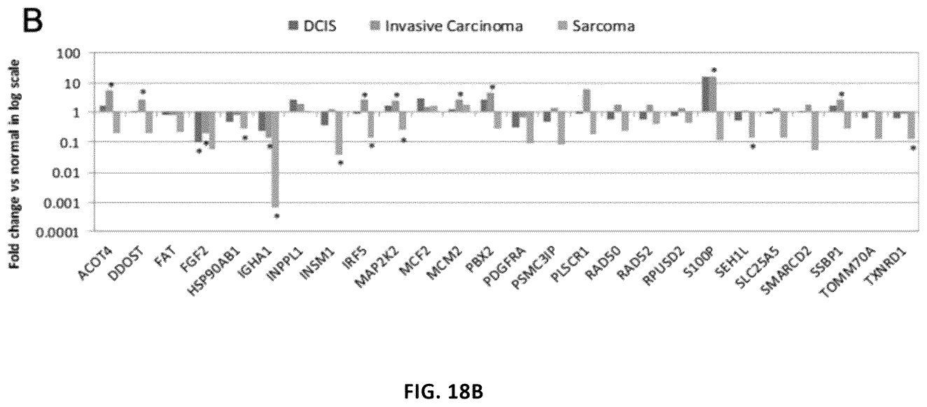

RT-PCR Validation of 26 Genes out of the 28-Gene Signature in Breast Cancer Tissue Samples. Microfluidic low-density arrays were designed to quantify the expression of 26 known genes in the gene signature in an independent patient cohort. Using the mRNA expression profiles of the 26 marker genes, 9 out of 12 normal breast tissue samples in the third patient cohort were separated from 65 breast cancer tumors in hierarchical clustering analysis (FIG. 18A), indicating its diagnostic capacity to classify breast cancer tumors from normal breast tissues in RT-PCR assays. Fourteen genes had significant (p<0.05) differential expression in breast cancer tumors vs. normal breast tissues. Specifically, ACOT4, DDOST, IRF5, MAP2K2, MCM2, PBX2, S100P, and SSBP1 had significant (p<0.05) upregulation in invasive carcinoma compared with normal breast tissues (FIG. 18B). FGF2 and IGHA1 had significant (p<0.05) downregulation in invasive carcinoma compared with normal breast tissues. FGF2 was also downregulated (p<0.05) in DCIS vs. normal breast tissues (FIG. 18B). Multiple genes were downregulated (p<0.05) in sarcoma tumors, including HSP90AB1, IGHA1, INSM1, IRF5, MAP2K2, and SEH1L (FIG. 18B). The protein expression of FGF-2, RAD52, RAD50, PBX2, MAP2K2 (MEK2), and S100P was also validated with immunohistochemistry in invasive breast cancer tumor tissues. RAD52 and PBX2 had differential protein expression among breast cancer stages.

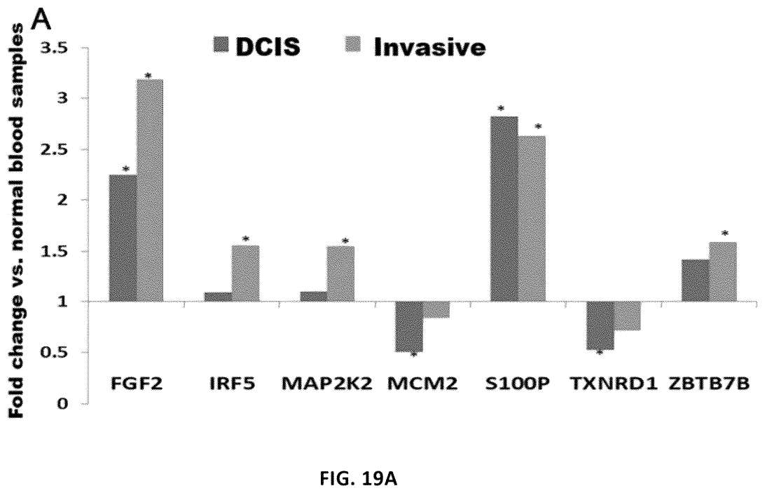

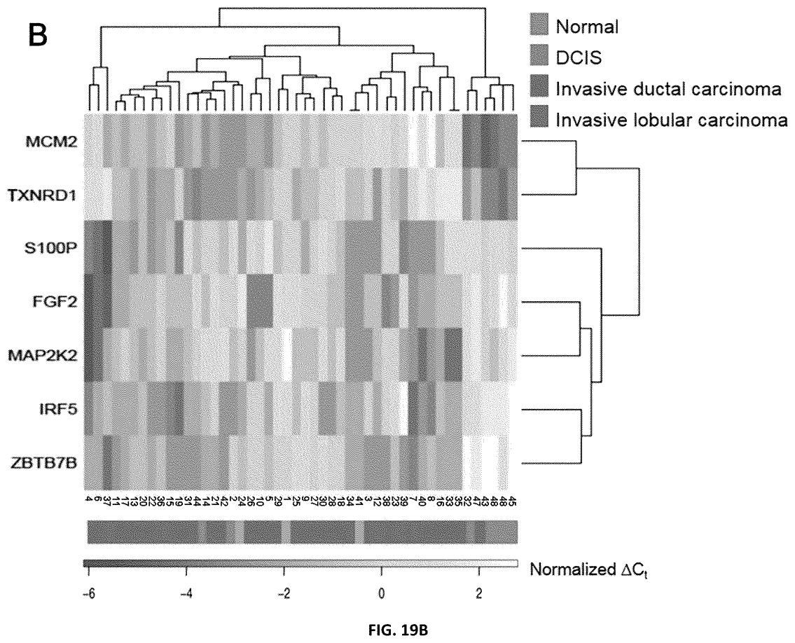

RT-PCR Validation of 28-Gene Signature in Breast Cancer Blood Samples. The mRNA expression of the 28-gene signature was further examined in breast cancer blood samples of the fifth patient cohort (n=48) using microfluidic low-density arrays in RT-PCR. Seven genes showed significant (p<0.05) differential expression in DCIS and/or invasive breast cancer patient blood samples compared with blood samples from healthy individuals. FGF2 and S100P were overexpressed (p<0.05) in both DCIS and invasive breast cancer patient blood samples. IRF5, MAP2K2 (MEK2), and ZBTB7B were significantly overexpressed (p<0.05) in invasive breast cancer blood samples but not in DCIS blood samples. MCM2 and TXNRD1 were underexpressed (p<0.05) in DCIS patient samples but not in invasive breast cancer samples (FIG. 19a). IRF5, MAP2K2 (MEK2), and S100P had concordant overexpressed mRNA in invasive breast cancer tissues and blood samples. Using the mRNA expression of these seven genes, normal blood samples were separated from breast cancer patient blood samples in hierarchical clustering analysis (FIG. 19B). The protein expression of RAD52, RAD50, PBX2, MAP2K2 (MEK2), and S100P was also validated with immunohistochemistry in invasive breast cancer tumor tissues). We acknowledge that there were only seven normal blood samples. This was a feasibility study that warrants a larger-scale analysis to substantiate its potential implications in the diagnosis of breast cancer in the future.

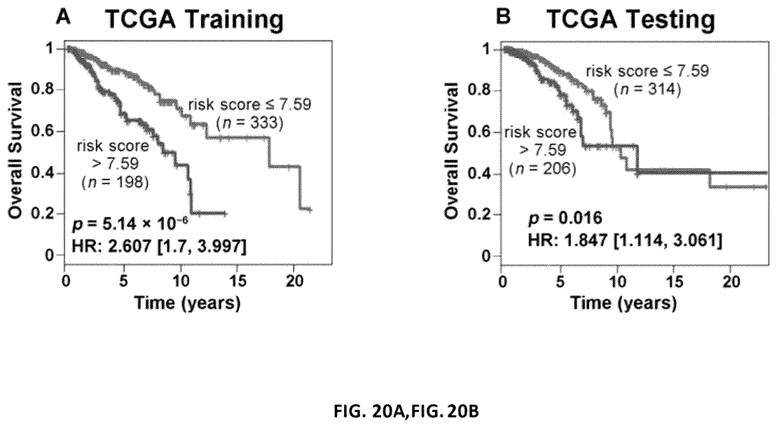

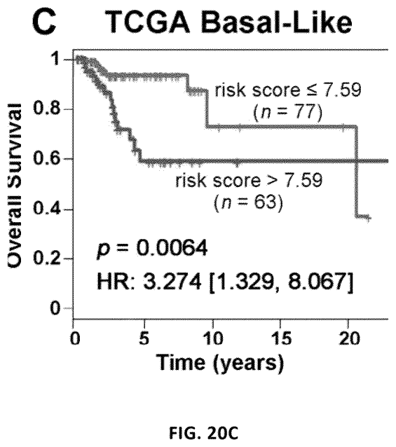

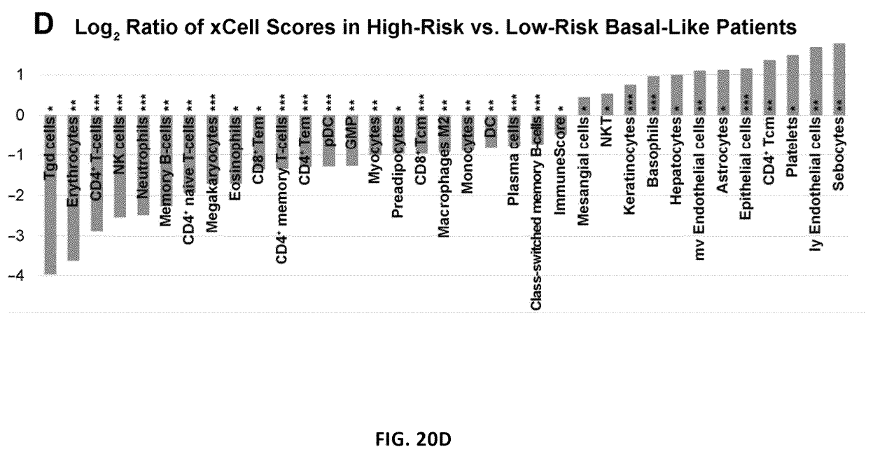

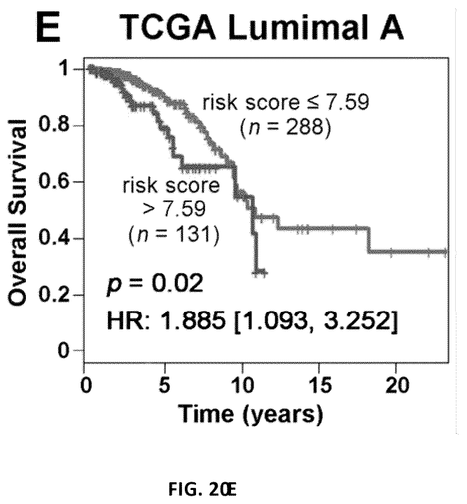

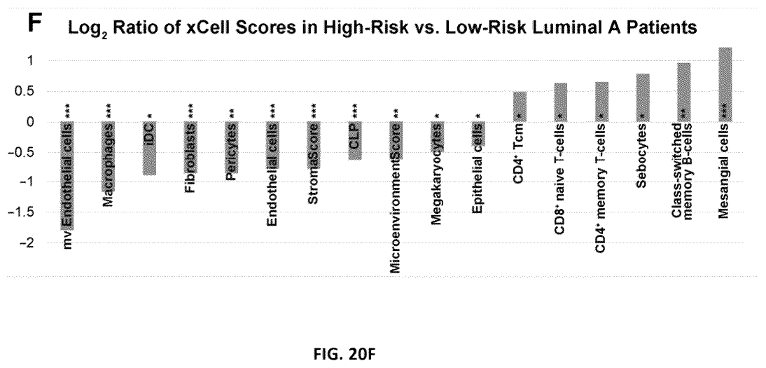

Prognosis within Breast Cancer Subtypes Using Next-Generation Sequencing Data. The Cancer Genome Atlas on breast cancer (TCGA-BRCA) dataset was randomly split into a training (n=547; 531 patients had sufficient survival information) and a testing set (n=548; 520 patients had sufficient survival information) to develop and validate the prognostic model. Using 25 available genes, a multivariate Cox model was constructed to compute the risk score for each patient. The same model was then applied to the testing set, employing identical gene coefficients and the cutoff for patient stratification. Kaplan-Meier analysis revealed that patients with a risk score less than or equal to 7.59 had significantly longer survival than patients with a risk score greater than 7.59 in both the training set (p=5.14×10-6, HR: 2.607 [1.7, 3.997]; FIG. 20A) and the testing set (p=0.016, HR: 1.847 [1.114, 3.061]; FIG. 20B).

Next, we evaluated the prognostic value of our model by performing survival analysis on four PAM50 subtypes in TCGA-BRCA patients. These subtypes include basal-like (n=140), luminal A (n=419), luminal B (n=180), and Her2-positive (n=62). Kaplan-Meier analysis demonstrated that the low-risk patient group had significantly better survival outcomes than the high-risk patient group in the basal-like (FIG. 20C) and luminal A subtypes (FIG. 20E). Specifically, the 6-year survival rates were 93.09% vs. 58.57% for low-risk vs. high-risk basal-like patients, respectively, and the 6-year survival rates were 86.88% vs. 67.9% for low-risk vs. high-risk luminal A patients, respectively. These results indicated that our prognostic model can further refine the prognosis within the basal-like and luminal A subtypes. In contrast, our prognostic model cannot further stratify the luminal B or Her2-positive subtypes. In the random split, we tried to keep the same proportion of samples in the training set (n=547) and the testing set (n=548). However, the specific number of each PAM50 subtype in the training set and the testing set will vary slightly in the random split. In the TCGA-BRCA cohort, luminal A has the largest number of patients (419 out of 1051) in the survival analysis. This might be the reason our classifier produced similar results for luminal A (FIG. 20E) and the testing set in the first 10 years after surgery (FIG. 20B).

For each patient sample in the TCGA-BRCA cohort, we computed the xCell [Aran, D.; Hu, Z.; Butte, A. J. xCell: Digitally portraying the tissue cellular heterogeneity landscape. Genome Biol. 2017, 18, 220. https://doi.org/10.1186/si3059-017-1349-1] scores, representing the transcriptional activities of all immune, epithelial, and stromal cell types using the corresponding RNA sequencing data. We identified immune cell types with significantly different activities in the low-risk vs. high-risk groups for the basal-like (FIG. 20D) and luminal A (FIG. 20F) subtypes. Cell type activity changes more in high-risk patients when the log 2 ratio is positive, whereas it varies more in low-risk patients when the log 2 ratio is negative. The following cell type signatures were more active in basal-like patients with a more favorable survival outcome: Tgd (gamma delta T) cells, erythrocytes, CD4+ T cells, NK cells, neutrophils, memory B cells, CD4+naïve T cells, megakaryocytes, eosinophils, CD8+ Tem (effector memory cells), CD4+ memory T cells, CD4+ Tem, plasmacytoid dendritic cells (pDC), granulocyte-macrophage progenitor (GMP), myocytes, dendritic cells (DC), plasma cells, class-switched memory B-cells, and ImmuneScore. The cell types that were more active in basal-like patients with a poor prognosis included the following: mesangial cells, NKT, keratinocytes, basophils, hepatocytes, my endothelial cells, astrocytes, epithelial cells, CD4+ Tcm (central memory cells), platelets, ly endothelial cells, and sebocytes. In luminal A patients with a good prognosis, the following cell types had increased variation in transcriptional activities defined with mRNA expression changes: my endothelial cells, macrophages, immature dendritic cells (iDC), fibroblasts, pericytes, endothelial cells, StromalScore, common lymphoid progenitor (CLP), MicroenvironmentScore, megakaryocytes, and epithelial cells. CD4+ Tcm, CD8+naïve T cells, CD4+ memory T cells, sebocytes, class-switched memory B-cells, and mesangial cells had more varied activities in luminal A patients with a poor prognosis.

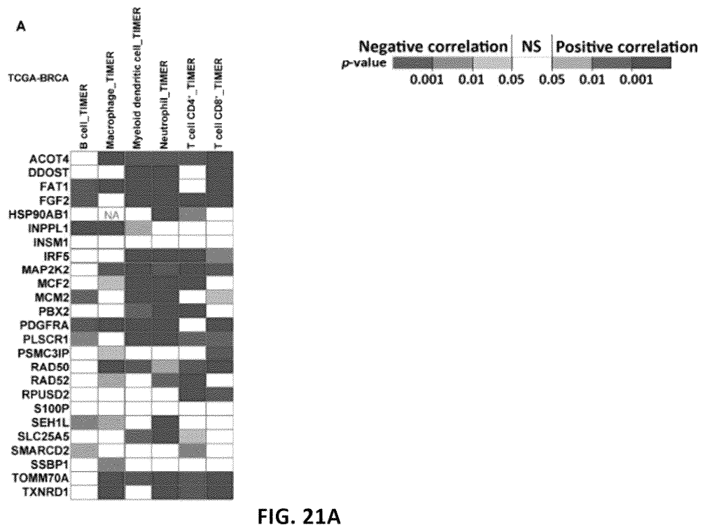

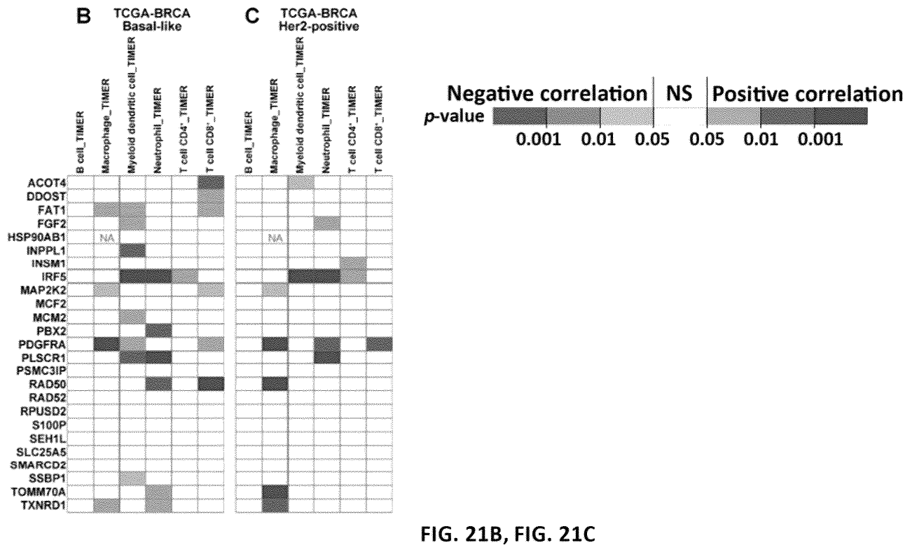

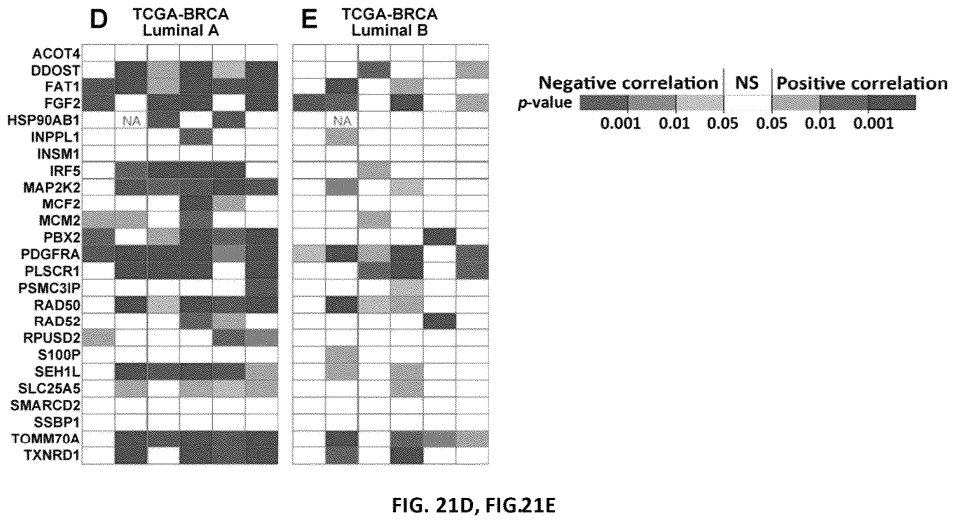

Correlation of Signature Genes with Immune Infiltration. We assessed the correlation between immune infiltration and the expression of 25 genes (FIG. 21; IGHA1 is not available, and FAT is named FAT1) in TCGA-BRCA patients (n=1100) using Tumor Immune Estimation Resource (TIMER) 2.0 [Li, T.; Fan, J.; Wang, B.; Traugh, N.; Chen, Q.; Liu, J. S.; Li, B.; Liu, X. S. TIMER: A Web Server for Comprehensive Analysis of Tumor-Infiltrating Immune Cells. Cancer Res. 2017, 77, e108-e110. https://doi.org/10.1158/0008-5472.CAN-17-0307, Li, T.; Fu, J.; Zeng, Z.; Cohen, D.; Li, J.; Chen, Q.; Li, B.; Liu, X. S. TIMER2.0 for analysis of tumor-infiltrating immune cells. Nucleic Acids Res. 2020, 48, W509-W514. https://doi.org/10.1093/nar/gkaa407]. The results revealed the immune infiltration of B cells, macrophages, myeloid dendritic cells, neutrophils, CD4+ T cells, and CD8+ T cells. The majority of our signature genes showed a significant correlation with immune infiltration in at least one of the examined cell types in luminal A patients or breast cancer patients with all subtypes combined (FIGS. 21A-F).

Protein Expression Validation of the Signature Genes Due to the limited sample size in our RT-PCR analysis, we correlated the RT-PCR results with SEER breast cancer patient data. This methodology correlates gene expression in a clinical cohort with large-scale SEER data based on a combined index of tumor grade and T, N, M in the cancer stage, as described in our previously published article [Ye, Q.; Putila, J.; Raese, R.; Dong, C.; Qian, Y.; Dowlati, A.; Guo, N. L. Identification of Prognostic and Chemopredictive microRNAs for Non-Small-Cell Lung Cancer by Integrating SEER-Medicare Data. Int. J. Mol. Sci. 2021, 22, 7658. https://doi.org/10.3390/ijms22147658]. This process can extrapolate gene expression into a larger patient population and examine the association with treatment outcomes. In this analysis, RAD52 and SMARCD2 had significant associations with outcomes in patients who received surgery only, radiation, or surgery plus radiation.

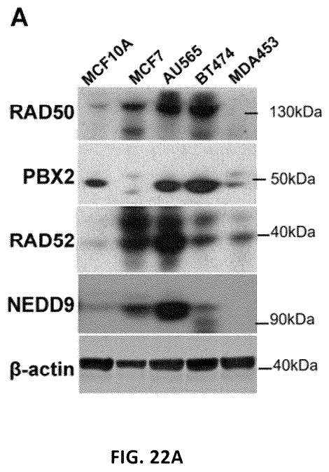

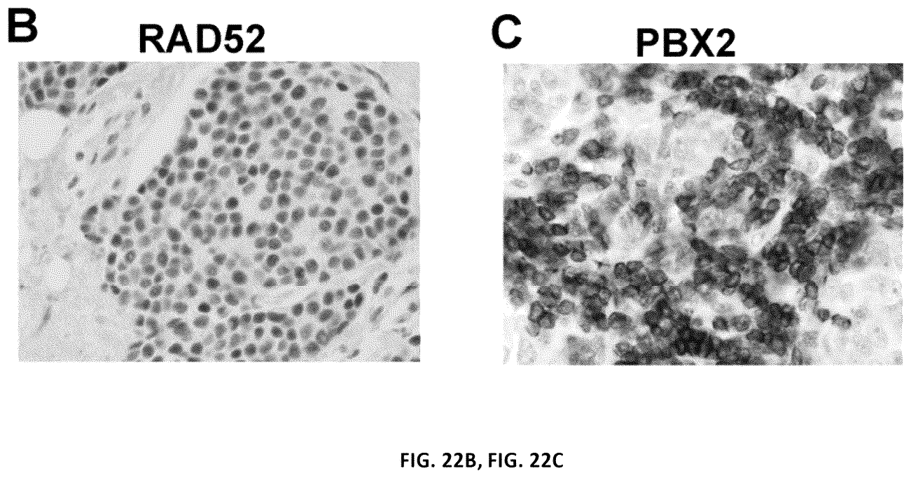

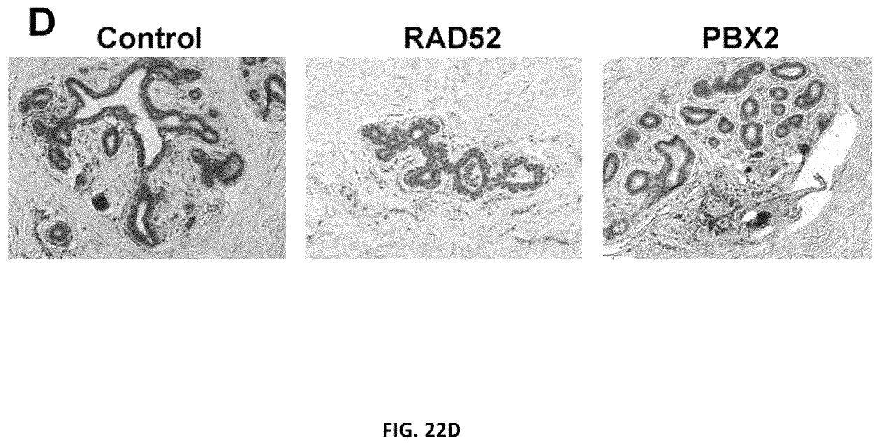

Based on these results, the protein expression of the signature genes was further validated using Western blots of cell lines and immunohistochemistry assays of tumor tissues in the fourth patient cohort to substantiate the functional involvement of the marker genes in breast cancer tumors. In Western blots, RAD50 was overexpressed in breast cancer cells (MCF7, AU565, and BT474) compared with normal breast epithelial cells (MCF10A; FIG. 22A). RAD50 was not expressed in TNBC cells MDA453. PBX2 was underexpressed in luminal A breast cancer MCF7 and TNBC MDA453 cells but was overexpressed in Her2-positive AU565 and BT474 cells [Smith, S. E.; Mellor, P.; Ward, A. K.; Kendall, S.; McDonald, M.; Vizeacoumar, F. S.; Vizeacoumar, F. J.; Napper, S.; Anderson, D. H. Molecular characterization of breast cancer cell lines through multiple omic approaches. Breast Cancer Res. 2017, 19, 65. https://doi.org/10.1186/si3058-017-0855-0], compared with normal breast MCF10A cells (FIG. 22A). The RAD52 protein was overexpressed in breast cancer cells (MCF7, AU565, BT474, and MDA453) compared with the immortalized breast cells (MCF10A; FIG. 22A), and its mRNA expression was not significantly different in breast cancer tumors compared with normal breast tissues (FIG. 18B). Both RAD50 and RAD52 are functionally involved in DNA repair [Symington, L. S. Role of RAD52 epistasis group genes in homologous recombination and double-strand break repair. Microbiol. Mol. Biol. Rev. MMBR 2002, 66, 630-670, table of contents. https://doi.org/10.1128/mmbr.66.4.630-670.2002], and their protein expression was largely correlated with the focal adhesion protein NEDD9 in breast cancer cells (FIG. 22A). NEDD9 (HEF1) plays an important role in cell attachment and division in mitosis [Pugacheva, E. N.; Golemis, E. A. The focal adhesion scaffolding protein HEF1 regulates activation of the Aurora-A and Nek2 kinases at the centrosome. Nat. Cell Biol. 2005, 7, 937-946. https://doi.org/10.1038/ncb1309], and promotes breast cancer development [Izumchenko, E.; Singh, M. K.; Plotnikova, O. V.; Tikhmyanova, N.; Little, J. L.; Serebriiskii, I. G.; Seo, S.; Kurokawa, M.; Egleston, B. L.; Klein-Szanto, A.; et al. NEDD9 promotes oncogenic signaling in mammary tumor development. Cancer Res. 2009, 69, 7198-7206. https://doi.org/10.1158/0008-5472.can-09-0795] and colorectal cancer progression [Li, Y.; Bavarva, J. H.; Wang, Z.; Guo, J.; Qian, C.; Thibodeau, S. N.; Golemis, E. A.; Liu, W. HEF1, a novel target of Wnt signaling, promotes colonic cell migration and cancer progression. Oncogene 2011, 30, 2633-2643. https://doi.org/10.1038/onc.2010.632]. The protein expression of RAD50, RAD52 (FIG. 22B), and PBX2 (FIG. 22C) was analyzed in breast cancer tumors using immunohistochemistry (IHC).

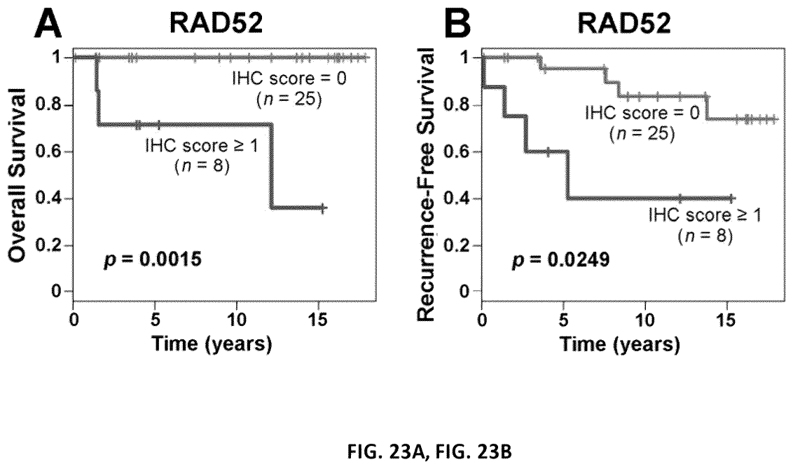

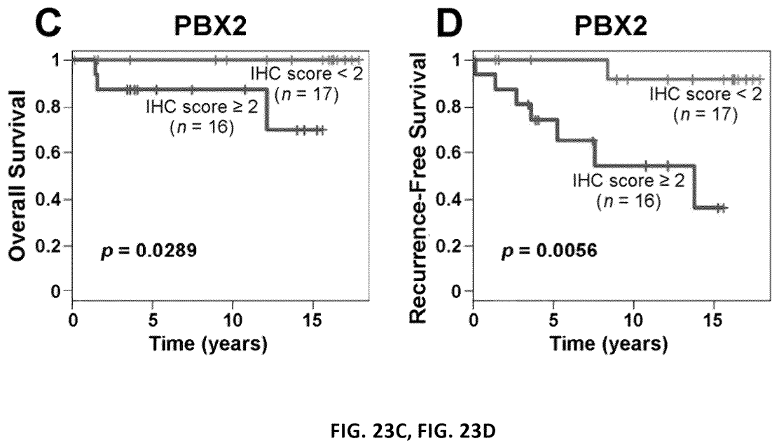

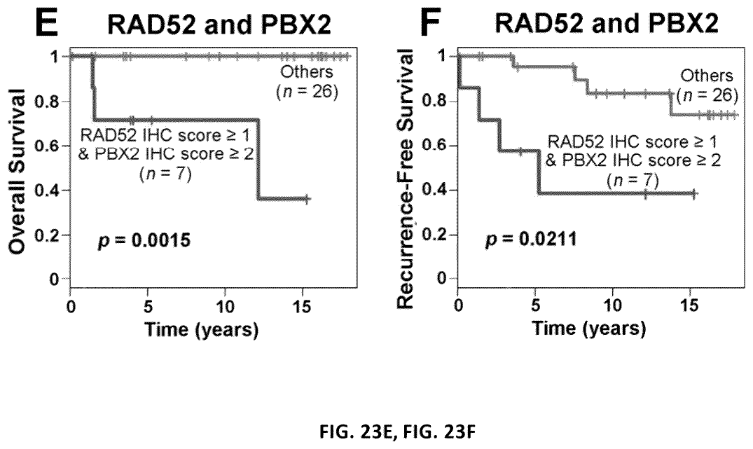

We performed further survival analyses based on the IHC results. The Kaplan-Meier results showed that the patients with higher IHC scores in RAD52 and/or PBX2 survived for a significantly shorter time than other patients (FIGS. 23A-F). SMARCD2 protein expression assessed with IHC was not prognostic in the examined breast cancer cohort.

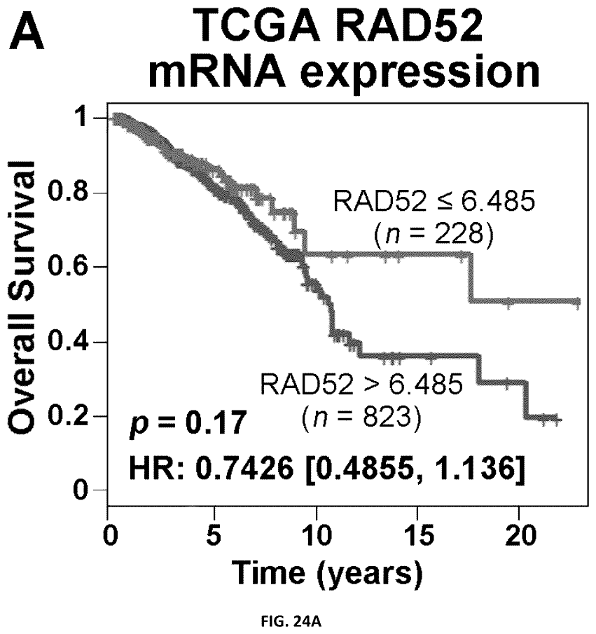

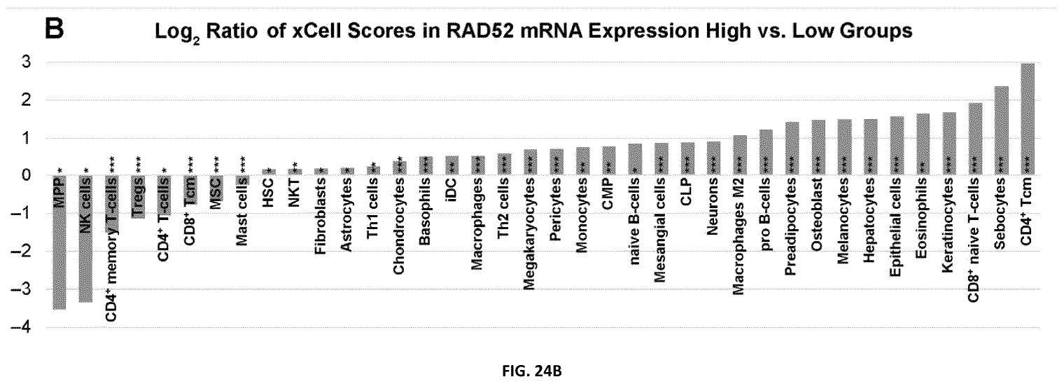

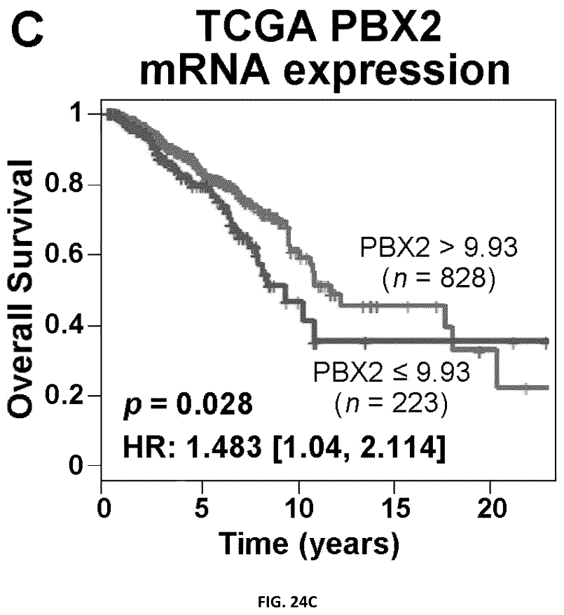

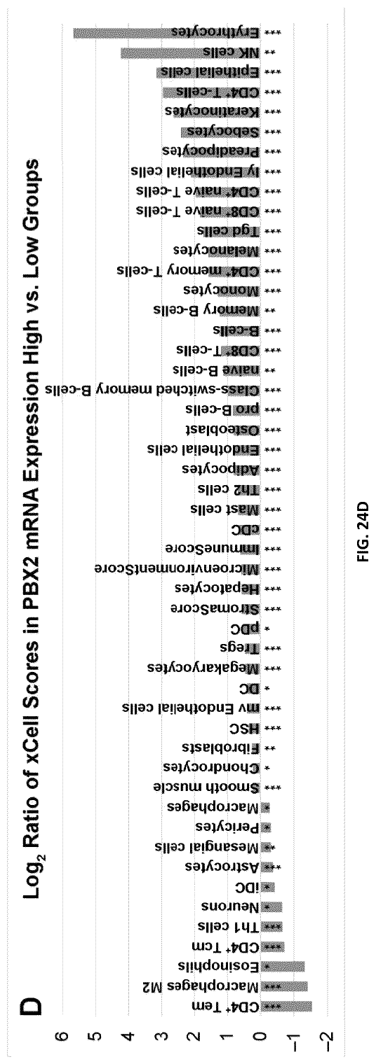

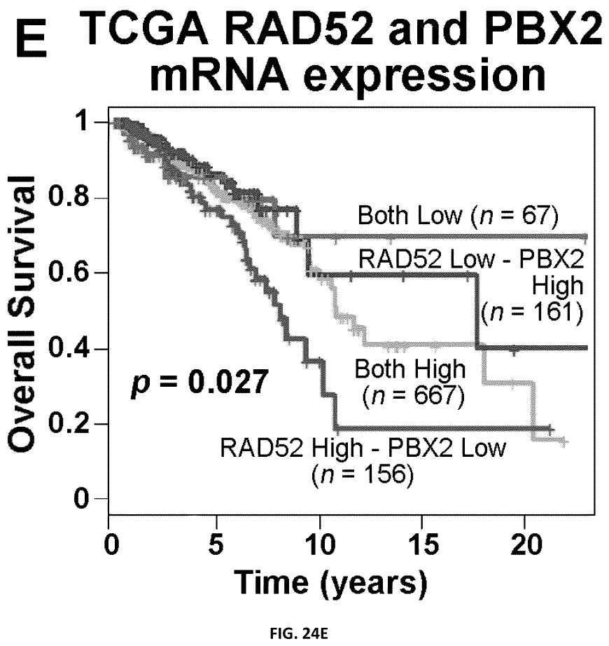

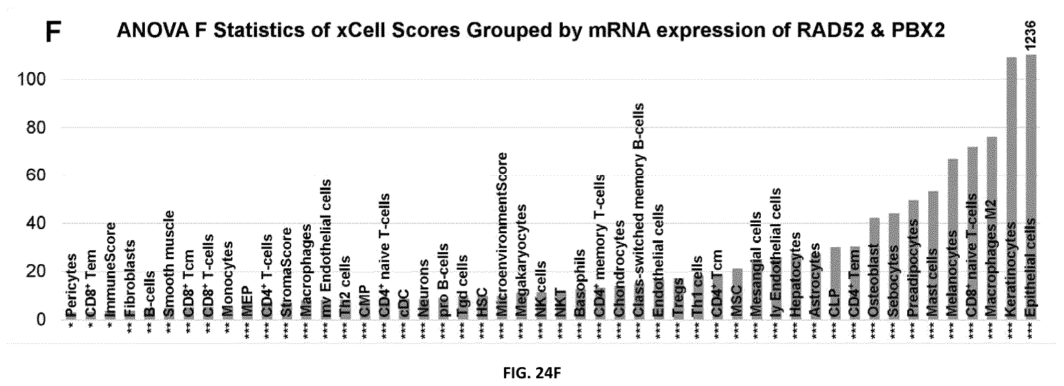

In the TCGA-BRCA patient cohort, the cutoffs of RAD52 and PBX2 mRNA expression were determined to have the most significant stratification (with the lowest p-value) in survival analysis. According to the Kaplan-Meier analysis of the TCGA-BRCA data, patients with a RAD52 mRNA expression level lower than 6.485 had a better prognosis for survival than those with a RAD52 mRNA expression level higher than 6.485 (FIG. 24A). Although the survival analysis result was not significant, the two groups had a good separation after 5 years. These results are consistent with the RT-PCR results in our patient cohort with the SEER breast cancer patients that higher RAD52 mRNA expression was associated with poor patient outcomes in those who received surgery only, radiation, or surgery and radiation. The mRNA and protein expression patterns of RAD52 were consistent. Unlike PBX2 protein expression, the patients with a PBX2 mRNA expression level higher than 9.93 had a better survival outcome than the patients with a PBX2 mRNA expression level lower than 9.93 in the TCGA-BRCA data (FIG. 24C). Based on the mRNA expression level of RAD52 and PBX2 together, the TCGA-BRCA patients were divided into four groups: both low (RAD52<6.485 and PBX2<9.93), both high (RAD52>6.485 and PBX2>9.93), RAD52 low—PBX2 high (RAD52<6.485 and PBX2>9.93), RAD52 high—PBX2 low (RAD52>6.485 and PBX2<9.93). The patients in the “both low” group had the best survival outcome among all four groups according to the Kaplan-Meier analysis results, which was consistent with their protein expression in the survival analyses (FIGS. 23E, 23F). The patients in the “RAD52 high—PBX2 low” group survived for the shortest length of time among the four groups (FIG. 24E).

RAD52 is functionally important in DNA repair, cancer susceptibility, and immunodeficiency [Ghosh, S.; Hönscheid, A.; Dückers, G.; Ginzel, S.; Gohlke, H.; Gombert, M.; Kempkes, B.; Klapper, W.; Kuhlen, M.; Laws, H. J.; et al. Human RAD52—A novel player in DNA repair in cancer and immunodeficiency. Haematologica 2017, 102, e69-e72. https://doi.org/10.3324/haematol.2016.155838]. PBX2, a homeobox gene, is 92% identical to human proto-oncogene PBX1 and is widely expressed in different states of differentiation and development [Monica, K.; Galili, N.; Nourse, J.; Saltman, D.; Cleary, M. L. PBX2 and PBX3, new homeobox genes with extensive homology to the human proto-oncogene PBX1. Mol. Cell. Biol. 1991, 11, 6149-6157. https://doi.org/10.1128/mcb.11.12.6149-6157.1991]. The Prep1/PBX2 complex regulates CCL2 expression, which is associated with numerous inflammatory diseases including HIV [Wright, E. K., Jr.; Page, S. H.; Barber, S. A.; Clements, J. E. Prep1/Pbx2 complexes regulate CCL2 expression through the −2578 guanine polymorphism. Genes Immun. 2008, 9, 419-430. https://doi.org/10.1038/gene.2008.33]. We sought to identify immune cell types that significantly (two-sample t-tests; p<0.05) differed in activity across breast cancer patient groups with high and low expression of RAD52 and PBX2 linked to their survival outcomes (FIGS. 24A, 24C). The log 2 ratios of xCell scores for tumors with high vs. low expression of RAD52 and PBX2 are shown in FIGS. 24B, 24D in the prognostic stratifications, respectively. For the four patient groups, a positive log 2 xCell score ratio of high- vs. low-expression groups showed that cell type activities varied more in high-expression tumors; a negative log 2 xCell score ratio of high- vs. low-expression groups showed that cell type activities varied more in low-expression tumors. The xCell scores were also computed for the four-group patient stratifications using combined RAD52 and PBX2 expression levels (FIG. 24E). Immune cell types with a significant difference in activities (ANOVA tests; F-statistics, p<0.05) among the four patient groups were identified (FIG. 24F).

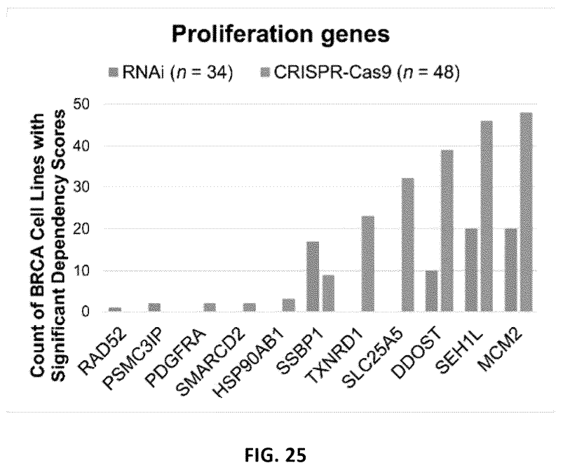

Proliferation potential of the Signature Genes. The functional role of the 28 signature genes in breast cancer cell proliferation was evaluated in publicly available high-throughput CRISPR-Cas9 (n 48) and RNAi (n 34) screening data for human BRCA cell lines. Available genes with a significant dependency score (<−0.5) in the BRCA cell lines are shown in FIG. 25, indicating that a knockdown/knockout of these genes significantly affects BRCA tumor cell growth in vitro. SSBP1, TXNRD1, SLC25A5, DDOST, SEH1L, and MCM2 had a significant effect in at least 50% of breast cancer cell lines in the CRISPR-Cas9 and/or RNAi screening data.

Association with Drug Response We found 18 NCCN-recommended regimens for preoperative/adjuvant/systemic/targeted treatment for breast cancer in the CCLE drug screening data. By utilizing the CCLE mRNA and proteomics profiles in human BRCA cell lines, we identified genes sensitive and resistant to these 18 drugs among 26 available signature genes (Table 9). Specifically, we classified genes as sensitive or resistant depending on whether they were significantly (p<0.05; two-sample t-tests) overexpressed in sensitive BRCA cell lines or resistant BRCA cell lines for a given drug. Pan-sensitive genes were defined as those that were sensitive to any of the 18 medicines under study and were not resistant to any of them. Likewise, the genes that were determined to be resistant to any of the 18 medicines and not sensitive to any of them were labeled as pan-resistant genes. HSP90AB1, INPPL1, IRF5, and RAD50 were pan-resistant genes. PLSCR1 and PBX2 were pan-sensitive genes at the mRNA expression level. FGF2 was resistant to alpelisib, and S100P was sensitive to olaparib in both mRNA and protein data.

Data from the Profiling Relative Inhibition Simultaneously in Mixtures (PRISM) and Genomics of Drug Sensitivity in Cancer (GDSC1/2) drug screening programs were included in our research.

TABLE 9
Resistant and sensitive genes to 18 drugs recommended in the NCCN Breast Cancer
guidelines, as determined using mRNA and proteomics data from CCLE human BRCA
cell lines. Genes with a significant association with mRNA expression are indicated
in italic font, and genes with significant results for protein expression are
indicated in normal font. Pan-resistant or pan-sensitive genes in terms of mRNA
expression are indicated in bold font. Genes with concordant mRNA and protein
expression association with drug response are in parenthesis. Genes with at least
one non-silent mutation in at least one BRCA cell line are underlined; genes
with fusions in BRCA cell lines are marked with an asterisk; genes with both
fusions and non-silent mutations in BRCA cell lines are in brackets.

| Drug | Resistant | Sensitive |
|---|---|---|
| 5-fluorouracil | HSP90AB1*, INPPL1, | DDOST |
| | TXNRD1, | |
| | FGF2 | |
| alpelisib | (FGF2), PSMC3IP, RAD50, | PLSCR1, SEH1L, |
| | S100P | [SMARCD2], |
| | | PBX2, S100P |
| capecitabine | HSP90AB1*, MCM2 | |
| carboplatin | | MAP2K2 |
| cisplatin | PDGFRA, PSMC3IP | DDOST, FGF2, PLSCR1, |
| | | SLC25A5, [SMARCD2], |
| | | MCM2 |
| cyclophosphamide | FGF2, MCM2 | MCM2 |
| docetaxel | INPPL1, IRF5, RAD52, | RPUSD2, S100P, SEH1L, |
| | [SMARCD2] | SLC25A5 |
| doxorubicin | | PDGFRA, PLSCR1, |
| | | PSMC3IP, RPUSD2, SEH1L, |
| | | SLC25A5, |
| | | MCM2 |
| fulvestrant | TXNRD1, | ACOT4, FGF2, INSM1, |
| | FGF2, S100P | MCM2, RPUSD2, S100P, |
| | | [SMARCD2] |
| gemcitabine | ACOT4, FGF2, INSM1, | MAP2K2, PSMC3IP, S100P, |
| | [SMARCD2] | SEH1L, SLC25A5, TXNRD1 |
| ixabepilone | | DDOST, PBX2, PDGFRA, |
| | | PLSCR1, RPUSD2 |
| lapatinib | INPPL1, INSM1, PSMC3IP, | [SMARCD2], |
| | S100P, SSBP1 | PBX2, S100P |
| methotrexate | HSP90AB1*, MAP2K2, | |
| | PBX2, RAD52 | |
| neratinib | PSMC3IP, RAD50, S100P, | PBX2 |
| | SLC25A5, TXNRD1 | |
| olaparib | FGF2 | FGF2, MAP2K2, PDGFRA, |
| | | RAD52, (S100P), SEH1L, |
| | | SLC25A5, SSBP1, TXNRD1, |
| paclitaxel | [SMARCD2], SSBP1, | SEH1L, SLC25A5 |
| | FGF2, MCM2 | |
| tucatinib | DDOST, PSMC3IP | FGF2, PBX2, RAD52 |
| vinorelbine | INPPL1 | DDOST, PBX2, PLSCR1, |
| | | PSMC3IP, RPUSD2, S100P, |
| | | SEH1L, SLC25A5, SSBP1, |
| | | TXNRD1 |

Discovery of New Drugs with CMap. After substantiating the associations with patient survival, drug response, and proliferation of our 28-gene signature, we sought to identify new drugs for treating breast cancer based on this gene expression signature. Among the 26 known signature genes, we defined downregulated genes as CMap input with the following criteria: (1) proliferation genes that had significant dependency scores in at least 50% of the tested BRCA cell lines in CRISPR-Cas9 (n=48) or RNAi (n=34); (2) survival hazard gene in TCGA-BRCA mRNA prognostic analysis (univariate Cox model p<0.05 and hazard ratio>1); (3) pan-resistant genes in mRNA expression data for the studied 18 drugs. The final downregulated genes were MCM2, SEH1L, SSBP1, DDOST, SLC25A5, TXNRD1, RAD50, TOMM70A, HSP90AB1, INPPL1, and IRF5.

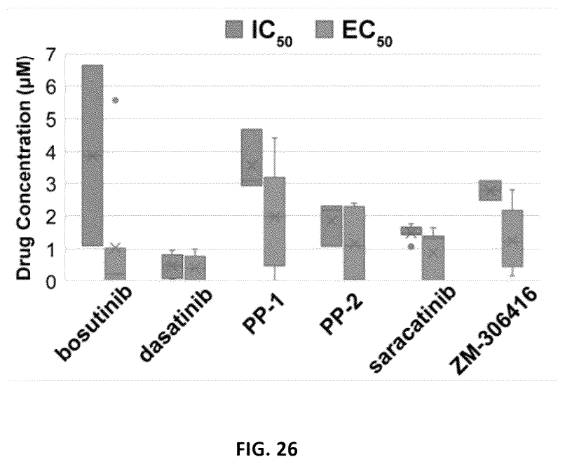

A total of 17 candidate new or repositioning medicines were found using CMap, along with the significantly enriched chemical sets (p<0.05, connectivity score>0.9). To find out if these candidate compounds can effectively inhibit the development of breast cancer cells, the half-maximal inhibitory concentration (IC50) and the half-maximal effective concentration (EC50) values of the drugs in the PRISM data were investigated in the CCLE human BRCA cell lines (n=22). Six drugs with small average measurement values may potentially inhibit the growth of BRCA cells with a dose believed to be safe. The average IC50 and EC50 values of these six drugs in the PRISM screening data are shown in FIG. 26.

For each discovered drug for treating breast cancer, we further identified resistant and sensitive genes associated with its drug response on a genome-wide scale. Dasatinib and PP2 had concordant sensitive/resistant results in mRNA and proteomics data, as shown in Table 10. We did not find sensitive/resistant genes with concordant mRNA and protein expression for the other four drugs. Non-silent mutations and fusions in human BRCA cell lines for the identified drug response genes were also revealed.

TABLE 10
Resistant and sensitive genes to dasatinib and PP-2 concordant
in mRNA and proteomics data in CCLE human BRCA cell lines.
Genes with at least one non-silent mutation in at least one
BRCA cell line are in bold; genes with fusions in BRCA cell
lines are marked by underline; genes with both fusions and
non-silent mutations in BRCA cell lines are in italic.

| Drug | Resistant Genes | Sensitive Genes |
|---|---|---|
| dasatinib | ACHE, AP3B2, CLRN3, | CNTN1, DHRS3, ERN2, |
| | GDAP1, LOXL1, MTMR6, | GATA6, LRP1B, TDRP |
| | PLCB1, RAB6B, TIGAR | |
| PP-2 | | PALM |

Discussion Breast cancer remains the most common cancer in women worldwide. Alongside inheritable genetic risk factors causing about 5-10% of breast cancer cases, the common modifiable risk factors include obesity, drinking alcoholic beverages [McDonald, J. A.; Goyal, A.; Terry, M. B. Alcohol Intake and Breast Cancer Risk: Weighing the Overall Evidence. Curr. Breast Cancer Rep. 2013, 5, 208-221. https://doi.org/10.1007/si2609-013-0114-z], and smoking [Johnson, K. C.; Miller, A. B.; Collishaw, N. E.; Palmer, J. R.; Hammond, S. K.; Salmon, A. G.; Cantor, K. P.; Miller, M. D.; Boyd, N. F.; Millar, J.; et al. Active smoking and secondhand smoke increase breast cancer risk: The report of the Canadian Expert Panel on Tobacco Smoke and Breast Cancer Risk (2009). Tob. Control 2011, 20, e2. https://doi.org/10.1136/tc.2010.035931]. The Gail model is currently used to estimate breast cancer risk based on demographic information. One study cautions healthcare professionals in counseling individual patients with atypia using the Gail model because the results show that the Gail model significantly underestimates the risk of breast cancer in women with atypia, and its ability to classify women with atypia into those who do and do not develop breast cancer is limited [Pankratz, V. S.; Hartmann, L. C.; Degnim, A. C.; Vierkant, R. A.; Ghosh, K.; Vachon, C. M.; Frost, M. H.; Maloney, S. D.; Reynolds, C.; Boughey, J. C. Assessment of the accuracy of the Gail model in women with atypical hyperplasia. J. Clin. Oncol. 2008, 26, 5374-5379. https://doi.org/10.1200/jco.2007.14.8833]. The Gail model also underestimates genetic risks in breast cancer [Sa-Nguanraksa, D.; Sasanakietkul, T.; Chayanuch, O.; Kulprom, A.; Pornchai, O. Gail Model Underestimates Breast Cancer Risk in Thai Population. Asian Pac. J. Cancer Prev. 2019, 20, 2385-2389. https://doi.org/10.31557/apjcp.2019.20.8.2385] and offers little improvement in breast cancer risk prediction in ER+ patients [Li, K.; Anderson, G.; Viallon, V.; Arveux, P.; Kvaskoff, M.; Fournier, A.; Krogh, V.; Tumino, R.; Sanchez, M. J.; Ardanaz, E.; et al. Risk prediction for estrogen receptor-specific breast cancers in two large prospective cohorts. Breast Cancer Res. 2018, 20, 147. https://doi.org/10.1186/s13058-018-1073-0]. Biomarker-based models revealing intrinsic molecular mechanisms underlying breast cancer are needed to improve its diagnosis, early detection, and intervention.

The past two decades have witnessed paradigm shifts in medicine, with ground-breaking discoveries from genomic studies and successful translation into clinical practices and health insurance policies. In the field of breast cancer, two multi-gene assays, Oncotype DX and MammaPrint, are commercially available for the prognosis of early-stage ER+ patients with invasive breast cancer in the US and Europe. Such advancement demonstrates the promise of utilizing genomic biomarkers for precision medicine. Recent research has utilized “multi-omics” technology including genome-scale DNA copy number variation (CNV), DNA methylation, mRNA, microRNA (miRNA), exome sequencing, and reverse-phase protein arrays to reveal the landscape of breast cancer and other cancer types, such as in The Cancer Genome Atlas (TCGA). Integrated CNV and mRNA sequencing analysis has been used to identify novel subtypes of breast cancer [Curtis, C.; Shah, S. P.; Chin, S. F.; Turashvili, G.; Rueda, O. M.; Dunning, M. J.; Speed, D.; Lynch, A. G.; Samarajiwa, S.; Yuan, Y.; et al. The genomic and transcriptomic architecture of 2,000 breast tumours reveals novel subgroups. Nature 2012, 486, 346-352. https://doi.org/10.1038/nature10983]. Differential expression of miR10b, miR34a, miR155, and miR195 was found in sera of breast cancer patients and healthy control individuals using qPCR [Hagrass, H. A.; Sharaf, S.; Pasha, H. F.; Tantawy, E. A.; Mohamed, R. H.; Kassem, R. Circulating microRNAs—A new horizon in molecular diagnosis of breast cancer. Genes Cancer 2015, 6, 281-287. https://doi.org/10.18632/genesandcancer.66]. Nevertheless, there are no clinically applied gene tests for breast cancer diagnosis in the general population.

Initially motivated to discover a prognostic genomic signature for the general breast cancer patient population, including patients with all cancer stages and ER/nodal status, we identified a 28-gene prognostic signature from a population-based breast cancer cohort and validated it in more than 2000 patients with all cancer stages and ER statuses. The prognostic capacity of the 28-gene signature is beyond early-stage, ER+/lymph node-negative breast cancer. In this study, we showed that this gene signature can further refine prognosis within the TNBC and luminal A BRCA subtypes and revealed immune cell types with distinct activity profiles linked to different prognostic groups within these subtypes. The majority of the signature genes had a significant association with immune infiltration in TCGA-BRCA patient tumors. This 28-gene signature is also prognostic of clinical outcomes in multiple epithelial cancers, including ovarian cancer.

In this study, we sought to investigate if 26 known genes of this 28-gene signature are potentially also diagnostic of breast cancer and able to identify those with a high potential for developing invasive breast cancer in patients with atypical ductal hyperplasia (ADH). The overall accuracy of discriminating normal breast tissue from breast cancer tumors was 94% and the accuracy was 100% in classifying patients with ADH who did not develop future cancer and those who did (ADHC). This gene signature was also validated in RT-PCR in an independent breast cancer cohort for separating normal breast tissues from breast cancer tumors including DCIS, invasive carcinoma, and sarcoma. The protein expression of multiple signature genes was validated in breast cancer and normal breast cell lines using Western blots and tumor tissues with immunohistochemistry. Using seven differentially expressed signature genes in blood samples of breast cancer patients versus healthy women in RT-PCR, the normal individuals were again separated from breast cancer patients, including DCIS and invasive carcinoma. These results show that it is feasible to use the 28-gene signature for early diagnostic detection using patient tissues and blood samples. If this gene assay can be validated in prospective evaluation in a larger patient cohort, it will have the potential utility in clinics to (1) advise the need for biopsy to confirm breast cancer diagnosis after the initial screening through using this minimally invasive blood-based gene assay; (2) estimate the potential for developing malignancy in patients diagnosed with benign lesions of ADH; (3) predict the likelihood for tumor recurrence/metastasis and aid the selection of specific therapy in patients diagnosed with invasive breast carcinoma. The presented protein expression patterns in this panel of genes reveal important information on their involvement in breast cancer initiation and progression. These results warrant large-scale validation in independent patient cohorts for clinical applications.

Among the genes with concordant mRNA and protein expression in the validation cohorts, MAP2K2 (MEK2), S100P, and PBX2 were all upregulated in terms of mRNA expression levels and had strong protein expression in invasive breast cancer tumors. MEK2 controls the activation of the MKK3/MKK6-p38 axis and has an essential impact on the MDA-MB-231 breast cancer cell survival and cyclin D1 expression [Huth, H. W.; Albarnaz, J. D.; Torres, A. A.; Bonjardim, C. A.; Ropert, C. MEK2 controls the activation of MKK3/MKK6-p38 axis involved in the MDA-MB-231 breast cancer cell survival: Correlation with cyclin Di expression. Cell. Signal. 2016, 28, 1283-1291. https://doi.org/10.1016/j.cellsig.2016.05.009]. Combined kinase inhibitors of MEK1/2 and either PI3K or PDGFR are efficacious in treating triple-negative breast cancer [Van Swearingen, A. E. D.; Sambade, M. J.; Siegel, M. B.; Sud, S.; McNeill, R. S.; Bevill, S. M.; Chen, X.; Bash, R. E.; Mounsey, L.; Golitz, B. T.; et al. Combined kinase inhibitors of MEK1/2 and either PI3K or PDGFR are efficacious in intracranial triple-negative breast cancer. Neuro Oncol. 2017, 19, 1481-1493. https://doi.org/10.1093/neuonc/nox052]. S100P by itself or together with Ezrin promotes the trans-endothelial migration of TNBC cells [Kikuchi, K.; McNamara, K. M.; Miki, Y.; Iwabuchi, E.; Kanai, A.; Miyashita, M.; Ishida, T.; Sasano, H. S100P and Ezrin promote trans-endothelial migration of triple negative breast cancer cells. Cell. Oncol. 2019, 42, 67-80. https://doi.org/10.1007/si3402-018-0408-2]. Consistent with our findings, other groups also reported the association of S100P mRNA [Zhang, S.; Wang, Z.; Liu, W.; Lei, R.; Shan, J.; Li, L.; Wang, X. Distinct prognostic values of S100 mRNA expression in breast cancer. Sci. Rep. 2017, 7, 39786. https://doi.org/10.1038/srep39786] and protein expression with worse breast cancer outcomes. In this study, both MEK2 and S100P were upregulated in blood and tumor samples of invasive breast cancer. S100P was also upregulated in DCIS patient blood samples, indicating its potential clinical utility as a non-invasive biomarker for breast cancer diagnosis by itself. Together with the previous findings reported in the literature, this study shows that our identified signature genes are diagnostic and prognostic in breast cancer and are functionally involved in tumorigenesis and progression.

PBX2 is also part of a 10-gene model to predict breast lesions identified in a whole-blood transcriptional profiling study [Hou, H.; Lyu, Y.; Jiang, J.; Wang, M.; Zhang, R.; Liew, C. C.; Wang, B.; Cheng, C. Peripheral blood transcriptome identifies high-risk benign and malignant breast lesions. PLoS ONE 2020, 15, e0233713. https://doi.org/10.1371/journal.pone.0233713]. PBX1 and PBX2 bind in a cooperative mechanism to DNA with both homeobox-containing genes HOXB7 and HOXB8 [van Dijk, M. A.; Peltenburg, L. T.; Murre, C. Hox gene products modulate the DNA binding activity of Pbx1 and Pbx2. Mech. Dev. 1995, 52, 99-108. https://doi.org/10.1016/0925-4773(95)00394-g, Neuteboom, S. T.; Peltenburg, L. T.; van Dijk, M. A.; Murre, C. The hexapeptide LFPWMR in Hoxb-8 is required for cooperative DNA binding with Pbx1 and Pbx2 proteins. Proc. Natl. Acad. Sci. USA 1995, 92, 9166-9170. https://doi.org/10.1073/pnas.92.20.9166]. The introduction of HOXB7 strongly increased the tumorigenic properties of breast cancer cells SkBr3 (SkBr3/B7) [67]. The functional requirement of the oncogenic activity of HOXB7 was proven with a dominant-negative PBX1 mutant, PBX1NT [67]. HOXB7 was shown to regulate the expression of Hox cofactors by increasing PBX2 and decreasing PBX1 in SkBr3 cells [Fernandez, L. C.; Errico, M. C.; Bottero, L.; Penkov, D.; Resnati, M.; Blasi, F.; Care, A. Oncogenic HoxB7 requires TALE cofactors and is inactivated by a dominant-negative Pbx1 mutant in a cell-specific manner. Cancer Lett. 2008, 266, 144-155. https://doi.org/10.1016/j.canlet.2008.02.042]. Interestingly, HOXB7 is functionally involved in tumor cell growth promotion through the direct transactivation of FGF2, which is also part of our 28-gene signature. FGF2 induces breast cancer growth by activating and recruiting ERa and PRBΔ4 isoforms to MYC regulatory sequences [Giulianelli, S.; Riggio, M.; Guillardoy, T.; Perez Piñero, C.; Gorostiaga, M. A.; Sequeira, G.; Pataccini, G.; Abascal, M. F.; Toledo, M. F.; Jacobsen, B. M.; et al. FGF2 induces breast cancer growth through ligand-independent activation and recruitment of ERα and PRBΔ4 isoform to MYC regulatory sequences. Int. J. Cancer 2019, 145, 1874-1888. https://doi.org/10.1002/ijc.32252]. The FGF2/FGFR1 paracrine loop is functionally involved in the crosstalk between breast cancer cells and tumor stroma. The activation of FGF2/FGFR1 paracrine signaling triggers the expression of the connective tissue growth factor (CTGF), causing the migration and invasion of MDA-MB-231 cells [Santolla, M. F.; Vivacqua, A.; Lappano, R.; Rigiracciolo, D. C.; Cirillo, F.; Galli, G. R.; Talia, M.; Brunetti, G.; Miglietta, A. M.; Belfiore, A.; et al. GPER Mediates a Feedforward FGF2/FGFR1 Paracrine Activation Coupling CAFs to Cancer Cells toward Breast Tumor Progression. Cells 2019, 8, 223. https://doi.org/10.3390/cells8030223]. The entire mechanism involving FGF2, PBX1/PBX2, and HOXB7/HOXB8 in breast cancer tumorigenesis is not clear. In this study, FGF2 mRNA was downregulated in DCIS and invasive breast cancer tumors compared with normal breast tissues but upregulated in blood samples from DCIS and invasive breast cancer patients versus healthy individuals. Underexpression of FGF2 mRNA and protein was also reported in malignant human breast tissues compared with non-malignant tissues [Yiangou, C.; Gomm, J. J.; Coope, R. C.; Law, M.; Lugmani, Y. A.; Shousha, S.; Coombes, R. C.; Johnston, C. L. Fibroblast growth factor 2 in breast cancer: Occurrence and prognostic significance. Br. J. Cancer 1997, 75, 28-33. https://doi.org/10.1038/bjc.1997.5]. The observed discrepancy of FGF2 mRNA expression between the patients' blood and tissues might be caused by its different functional involvements in breast cancer tissues and blood.

In this study, we examined the protein expression of 28 marker genes with available antibodies, including MCF2, RAD52, RAD50, PBX2, SMARCD2, IRF5, MCM2, and IGAH2, in Western blots. The protein expression of RAD50, RAD52, and PBX2 was confirmed in breast cancer cell lines MCF7, AU565, BT474, and MDA453 and immortalized breast epithelial cell line MCF10A [Chavez, K. J.; Garimella, S. V.; Lipkowitz, S. Triple negative breast cancer cell lines: One tool in the search for better treatment of triple negative breast cancer. Breast Dis. 2010, 32, 35-48] (FIG. 22A). The results for the rest of the markers without any detectable protein expression in Western blots are not shown. In the future, we will further examine the protein expression of the marker genes in different breast cancer tumors to investigate their functions.

Based on the association of the 28-gene expression signature with BRCA patient survival, response to 18 NCCN-recommended drugs, and proliferation, we designed mechanisms of action to inhibit BRCA cell growth, survival hazard genes, and pan-resistant genes to discover potential new drugs or new indications of existing drugs for treating breast cancer. The candidate compounds screened from CMap were further selected based on their efficacy in inhibiting BRCA cell growth measured with IC50 and EC50. Among the six identified drugs, bosutinib is a small molecule functioning as a dual inhibitor of BCR-ABL and Src tyrosine kinase for treating chronic myeloid leukemia (CML) and advanced solid tumors [Keller, G.; Schafhausen, P.; Brummendorf, T. H. Bosutinib. Recent Results Cancer Res. 2010, 184, 119-127. https://doi.org/10.1007/978-3-642-01222-8_9], including estrogen-positive breast cancer [Singh, P.; Singh, N.; Mishra, N.; Nisha, R.; Alka; Maurya, P.; Pal, R. R.; Singh, S.; Saraf, S. A. Functionalized bosutinib liposomes for target specific delivery in management of estrogen-positive cancer. Colloids Surf. B Biointerfaces 2022, 218, 112763. https://doi.org/10.1016/j.colsurfb.2022.112763]. Dasatinib is an oral inhibitor of multiple tyrosine kinases, including BCR-ABL, Src, c-KIT, PDGFR-α, PDGFR-β, and ephrin receptor kinases [Lindauer, M.; Hochhaus, A. Dasatinib. Recent Results Cancer Res. 2018, 212, 29-68. https://doi.org/10.1007/978-3-319-91439-8_2]. Dasatinib is effective for treating CML and Philadelphia chromosome-positive acute lymphoblastic leukemia (Ph+ ALL). A combination of paclitaxel and dasatinib showed some clinical activity in HER2-negative metastatic breast cancer in a phase II study [Morris, P. G.; Rota, S.; Cadoo, K.; Zamora, S.; Patil, S.; D'Andrea, G.; Gilewski, T.; Bromberg, J.; Dang, C.; Dickler, M.; et al. Phase II Study of Paclitaxel and Dasatinib in Metastatic Breast Cancer. Clin. Breast Cancer 2018, 18, 387-394. https://doi.org/10.1016/j.clbc.2018.03.010]. Dasatinib induced sensitivity to c-Met inhibition in TNBR cells in a pre-clinical study [Gaule, P.; Mukherjee, N.; Corkery, B.; Eustace, A. J.; Gately, K.; Roche, S.; O'Connor, R.; O'Byrne, K. J.; Walsh, N.; Duffy, M. J.; et al. Dasatinib Treatment Increases Sensitivity to c-Met Inhibition in Triple-Negative Breast Cancer Cells. Cancers 2019, 11, 548. https://doi.org/10.3390/cancers11040548]. Src inhibitor PP-1 suppresses the invasiveness of breast cancer cells [Xu, H.; Washington, S.; Verderame, M. F.; Manni, A. Role of non-receptor and receptor tyrosine kinases (TKs) in the antitumor action of alpha-difluoromethylornithine (DFMO) in breast cancer cells. Breast Cancer Res. Treat. 2008, 112, 255-261. https://doi.org/10.1007/s10549-007-9866-3]. PP-1 and PP-2 inhibit breast cancer proliferation [Shim, H. J.; Kim, H. I.; Lee, S. T. The associated pyrazolopyrimidines PP1 and PP2 inhibit protein tyrosine kinase 6 activity and suppress breast cancer cell proliferation. Oncol. Lett. 2017, 13, 1463-1469. https://doi.org/10.3892/ol.2017.5564]. Another Src inhibitor, saracatinib, is in clinical trials for treating multiple solid tumors, including breast cancer and lung cancer [Roskoski, R., Jr. Src protein-tyrosine kinase structure, mechanism, and small molecule inhibitors. Pharmacol. Res. 2015, 94, 9-25. https://doi.org/10.1016/j.phrs.2015.01.003]. The VEGFR inhibitor ZM-306416 was discovered as an EGFR inhibitor [Antczak, C.; Mahida, J. P.; Bhinder, B.; Calder, P. A.; Djaballah, H. A high-content biosensor-based screen identifies cell-permeable activators and inhibitors of EGFR function: Implications in drug discovery. J. Biomol. Screen. 2012, 17, 885-899. https://doi.org/10.1177/1087057112446174]. We previously discovered it as a new drug for treating lung cancer. See Ye, Q.; Hickey, J.; Summers, K.; Falatovich, B.; Gencheva, M.; Eubank, T. D.; Ivanov, A. V.; Guo, N. L. Multi-Omics Immune Interaction Networks in Lung Cancer Tumorigenesis, Proliferation, and Survival. Int. J. Mol. Sci. 2022, 23, 14978, which is incorporated herein by reference. In this study, it was discovered as a potential new drug for treating breast cancer that was not known before.

Patient Samples. The first cohort contained 185 breast tissue samples. Gene expression was quantified using the Affymetrix U133 Plus 2 Array (GEO accession number GSE10780) from Chen et al. These 185 samples include 143 histologically normal breast tissue samples and 42 invasive ductal carcinomas (IDC) tissue samples collected from 90 breast cancer patients who underwent a mastectomy. Each mastectomy specimen, where feasible, was prosected to produce an IDC and up to five successively derived, adjacent normal tissue samples from the ipsilateral breast or the four quadrants of the contralateral breast. All 143 histologically normal breast tissue samples were confirmed to be free of atypical ductal hyperplasia (ADH) and in situ or invasive breast carcinoma by one breast pathologist.

The second cohort contained 8 patient samples with gene expression quantified using the Affymetrix U133A microarray (GEO accession number GSE2429) from Poola et al. Among the 8 samples, 4 tissue samples were ADHC (from patients with ADH who had cancer concurrently, who had cancer before diagnosis of ADH, or who developed cancer subsequently) and 4 samples were from patients with ADH who had no prior history of breast cancer and had not developed breast cancer in five years after diagnosis.

The third cohort contained 77 snap-frozen breast tissue samples, including 3 ductal carcinomas in situ (DCIS), 60 invasive breast carcinomas, 2 sarcomas of the breast, and 12 normal breast tissues. These samples were obtained from the West Virginia University (WVU) Tissue Bank or Cooperative Human Tissue Network (CHTN) operated by the US National Cancer Institute. Tumor tissues were collected in surgical resections and were snap-frozen at −80° C. until RNA extraction. This cohort was used for quantitative RT-PCR validation of the biomarkers.

The fourth cohort contained 33 paraffin-embedded breast cancer tumor specimens used for immunohistochemistry assays. These samples were obtained from the WVU Tissue Bank. Histological preparations of tumor sections were examined by pathologists. This study was approved with an IRB exemption from WVU to use de-identified patient samples.

The fifth cohort contained 48 blood samples collected from patients seen at the Betty Puskar Breast Center of the WVU Cancer Institute/Mary Babb Randolph Cancer Center. The blood samples had been collected by the WVU Biospecimen Core and Tissue Bank. The blood samples were drawn from normal individuals (n=7), or patients diagnosed with breast cancer (n=41), including DCIS (n=3) and invasive breast cancer (n=38), with the patient's consent. No patients received any chemotherapy or radiation within the 6 months prior to the blood draw (but they were eligible if on either Herceptin and/or anti-hormone therapy).

The sixth cohort contained data of log 2-transformed Illumina HiSeq sequencing samples (n=1093) and reverse-phase protein array (RPPA) expression normalized samples (n=886) of The Cancer Genome Atlas on breast cancer (TCGA-BRCA). The datasets were retrieved from the online platform LinkedOmics [82] (http://www.linkedomics.org/, accessed on 10 Mar. 2023).

Blood Collection. Blood for research was drawn under sterile conditions using the BD Vacutainer Safety-Lok Blood Collection Set and the One-Use Holder through venipuncture into PAXgene® Blood RNA Tubes (Qiagen, Germantown, MD, USA) by a certified phlebotomist after the patient's consent was obtained. PAXgene tubes were collected following the protocol to allow at least 10 s for a complete blood draw to take place or till the blood had stopped flowing into the tube (˜2.5 mL of blood). After gently mixing by inverting the PAXgene Blood RNA Tubes 8 to 10 times, the blood samples were stored at 4° C. overnight.

RNA Extraction, Quality, and Concentration Assessment. Total RNA was extracted from 77 frozen breast tissue samples using the RNeasy mini kit according to the manufacturer's protocol (Qiagen, Germantown, MD, USA). RNA was eluted in 30 μL of RNase-free water and stored at −80° C. Total RNA was extracted from 48 blood samples. Blood samples were stored in PAXgene blood tubes (Qiagen, Germantown, MD, USA), which were equilibrated to room temperature for 2 h prior to isolation. Whole-blood RNA isolation was carried out using the PAXgene Blood miRNA Kit (Qiagen, Germantown, MD, USA) following the standard protocol. RNA was eluted with 40 μL Buffer BR5 directly onto the spin column membrane at 20,000 g twice. RNA was denatured by incubating for 5 min at 65° C. and then stored at −80° C. The quality and integrity of the total RNA, 28S/18S ratio, and a visual image of the 28S and 18S bands were evaluated on the 2100 Bioanalyzer RNA 6000 Nano LabChip (Agilent Technologies, Santa Clara, CA, USA). The concentration of the RNA was assessed using the Nanodrop 1000 Spectrophotometer (NanoDrop Technologies, Wilmington, DE, USA).

Generation of Complementary DNA (cDNA). Only high-quality RNA extracted from breast cancer samples was selected for conversion to cDNA. Reverse transcriptase polymerase chain reaction was used to convert single-stranded RNA to double-stranded cDNA using a Techne® TC-312 PCR instrument (MIDSCI, St. Louis, MO, USA). For standardization across all samples, one microgram of RNA was used to generate cDNA.

Real-Time RT-PCR Low-Density Arrays. Two TaqMan Low-Density Array endogenous control gene cards (Applied Biosystems/Thermo Fisher Scientific Corporation, Waltham, MA, USA) were run on an Applied Biosystems PRISM 7900HT Sequence Detection System for 16 breast cancer tumor samples to identify genes that had the most relatively constant expression in different tissue samples. Four control genes, namely 18S, GAPDH, HMBS, and IP08, had constant expression in breast cancer tissue samples. Constant mRNA expression of 18S, HMBS, and IP08 genes was also confirmed for all breast tissue samples using the individual TaqMan® Gene Expression Assays. We used 384-well microfluidic low-density array plates designed to contain these 4 endogenous control genes and 26 breast cancer marker genes with 2 unknown genes removed from the original 28-gene signature. The average expression of the four control genes was more stable than some single genes, but it was not the most stable compared to the best control gene in the analysis of breast cancer subtypes, stages, grades, or primary vs. recurrent samples. In the final analysis, 18S was used as the control gene in the analysis of breast cancer samples vs. normal samples, and HMBS was used as the control genes of the analysis across breast cancer stages, grades, and primary vs. recurrent breast cancer samples.

The mRNA expression of the 26 signature genes was measured in each of the breast cancer tumors and normal breast tissues through RT-PCR using TaqMan® Gene Expression Assays on a 7900 HT Fast RT-PCR instrument (Applied Biosystems/Thermo Fisher Scientific Corporation, Waltham, MA, USA). On each plate, 4 patient samples were loaded, and each primer was measured in triplicates. The report generated using Applied Biosystems SDS2.3 software included the number of cycles required to reach threshold fluorescence (Ct) and relative quantification (RQ), which numerically defined the expression pattern for the genes.

Statistical Analysis of Real-Time RT-PCR Data. ΔCt represents the normalized gene expression relative to the control gene HMB S in that sample. The average ΔCt was calculated for each tumor type, including ductal carcinoma in situ, sarcoma, and invasive breast carcinoma. The fold change of each gene marker was computed for each tumor type vs. normal breast tissue samples. All statistical analysis was based on ΔCt values, using two-tailed unpaired t-tests. p<0.05 was considered statistically significant.

Stromal and Immune Infiltration. xCell [41] is a computational tool that predicts the relative abundance of immune and stroma cell types in complex tissue samples using gene expression data. The tool is based on a novel gene signature-based method that enables accurate quantification of 64 immune and stroma cell types, including T cells, B cells, natural killer cells, macrophages, fibroblasts, and endothelial cells. The xCell R package version 1.1.0 was used for the analysis in this study.

To analyze the immune microenvironment of patient samples, xCell scores were calculated using the single-sample gene set enrichment analysis (ssGSEA) method. High xCell scores indicate that the corresponding cell type is present in varying levels across the samples, whereas low xCell scores indicate that the cell type is present in similar levels across all the samples.

The association between gene expression and immune infiltration was determined using TIMER 2.0, which is an updated version of the Tumor Immune Estimation Resource (TIMER). Accessible via its website at http://timer.cistrome.org/(accessed on 19 Mar. 2023), TIMER 2.0 is a valuable web server and database that offers researchers a comprehensive resource for investigating immune cell infiltrates in various types of cancer, including B cells, macrophages, myeloid dendritic cells, neutrophils, CD4+ T cells, and CD8+ T cells. The TCGA-BRCA cohort [Vasaikar, S. V.; Straub, P.; Wang, J.; Zhang, B. LinkedOmics: Analyzing multi-omics data within and across 32 cancer types. Nucleic Acids Res. 2018, 46, D956-D963. https://doi.org/10.1093/nar/gkx1090] was analyzed with xCell and TIMER2.0.

Protein Expression Validation Using Western Blot Analysis. Anti-PBX2 (sc-101853), -RAD50 (sc-20155), and -RAD52 (sc-8350) antibodies were obtained from Santa Cruz Biotechnology (Santa Cruz, CA, USA). Anti-MCF2 was obtained from Cell Signaling Technology (Danvers, MA, USA). Anti-IGHA2 was ordered from Abnova (Walnut, CA, USA). Anti-SMARCD2, -IRF5, and -MCM2 were ordered from AbCam (Cambridge, MA, USA). Anti-NEDD9 (HEF1) is a custom-made antibody. β-Actin (13E5) Rabbit mAb #4970 was ordered from Cell Signaling Technology (Danvers, MA, USA). A protein extraction kit was ordered from EMD (Gibbstown, NJ, USA). Western blot analysis was performed on normal breast cells (MCF10A) and breast cancer cells (MDA-MB-231) according to the methods described previously [83]. Specifically, MCF10A and MDA-MB-231 cells were cultured in MEGM and DMEM, respectively, with 10% fetal bovine serum and 5% CO2 at 37° C. The cells were lysed, and the lysates were subjected to SDS-PAGE, followed by immunoblotting with anti-PBX2, -RAD50, and -RAD52 antibodies, respectively. The remaining antibodies were not detected in the tested breast cancer cells in the Western blot analysis.

Protein Expression Validation Using Immunohistochemistry Analysis Histologic slides containing tumor tissue from 33 patients were stained using the Ventana BenchMark Auto Stainer along with normal breast tissue from cancer patients, which was used as the control. The following is a brief outline of the protocol used on the auto-stainer: (1) histologic sections were deparaffinized and antigen retrieval was performed through incubation in Cell Conditioning (CC1) Solution for 30 min; (2) the primary antibody (rabbit polyclonal anti-MEK2; AbCam Inc Cambridge, MA, USA; Catalogue #ab28834) was applied at a dilution of 1:50 for 60 min; (3) the slides were incubated for 18 min with a second antibody; and (4) counterstained with hematoxylin. Likewise, anti-S100P (Proteintech Group Inc, Rosemont, IL, USA; Catalog #11803-1-AP) and anti-FGF2 (Santa Cruz Biotechnology, Santa Cruz, CA, USA; Catalog #sc79) were used. Human colon cancer tissue was used as a positive control. The following protocol was used: (1) slides from the same 33 patients were deparaffinized and antigen retrieval was performed using CC1 for 30 min; (2) the slides were incubated with the primary antibody for 32 min at a dilution of 1:200 and the second antibody for 20 min; and (3) the slides were counterstained with hematoxylin. Anti-PBX2, -RAD50, and -RAD52 antibodies used in Western blots were also applied in immunohistochemistry assays of the same 33 patient samples. The protein expression scores in immunohistochemistry were quantified by a certified pathologist in the range of 0 to 4 as follows: 0=no staining; 1=equivocal staining; 2=weak staining; 3=moderate staining; 4=strong staining.

Microarray Data Processing. Due to the differences in microarray platforms, 26 signature genes were found in GSE10780, and 25 signature genes were found in GSE2429. For the genes with multiple matching probes, the median expression of the duplicates was used to represent the gene expression in building the classifiers.

Construction of Molecular Classifiers. A regression algorithm with a model tree (M5P) as the base learner was used to build classifiers based on the 28-gene signature using the software WEKA 3.4 [Witten, I. H.; Frank, E. Data Mining: Practical Machine Learning Tools and Techniques, 2nd ed.; Morgan Kaufmann: Burlington, MA, USA, 2005]. The model tree algorithm is based on the decision tree, in which each leaf stores a linear regression model that predicts the class value of instances that reach the leaf. Thus, predictions from the model tree are continuous numerical values. In this study, the classification of disease states was performed with a regression algorithm that constructed a model tree for each class, and instances were categorized into the class with the larger predicted value from the model tree.

Time-Dependent Receiver Operating Characteristics (ROC) Curves and Area under the ROC Curve (AUC). To evaluate the predictive performance of the proposed survival gene signatures, we employed time-dependent ROC analysis for censored data and AUC (area under the ROC curve) as our criteria to assess the 5-year survival predictions [Heagerty, P. J.; Lumley, T.; Pepe, M. S. Time-dependent ROC curves for censored survival data and a diagnostic marker. Biometrics 2000, 56, 337-344]. The time-dependent sensitivity and specificity functions are defined as follows:

$$sensitivity ( c , t ) = P \{ X > c \vert D ( t ) = 1 \}$$

$$specificity ( c , t ) = P \{ X \leq c \vert D ( t ) = 0 \}$$

The corresponding ROC(t) curve for any time t is defined as the plot of sensitivity (c, t) versus 1-specificity (c, t), with cutoff point c varying. X is the covariate and D(t) is the event indicator (here, death) at time t. The area under the curve, AUC(t), is defined as the area under the ROC(t) curve. The nearest neighbor estimator for the bivariate distribution function is used for estimating these conditional probabilities accounting for possible censoring [Akritas, M. G. Nearest neighbor estimation of a bivariate distribution under random censoring. Annu. Stat. 1994, 22, 1299-1327]. AUC can be used as an accuracy measure of the diagnostic marker; the larger the AUC the better the prediction model. An AUC equal to 0.5 indicates no predictive power, while an AUC equal to 1 represents perfect predictive performance. The analysis was performed using the software package R version 4.3.1.

Clustering Analysis. The cluster analysis was performed by using the Heatplus package in R version 4.3.1. The Euclidean method was used to compute the distance and the agglomeration method was “average”. Dendrograms were plotted using within-gene-scaled ΔCt values.

Proliferation Assays. In this study, we aimed to identify the proliferation determinants of breast tumor cells by analyzing the genome-scale CRISPR knockout and RNAi knockdown screening data of breast cancer cell lines. The CRISPR knockout screening results consisted of Achilles (Avana Cas9 library) [Dempster, J. M. R. J.; Kazachkova, M.; Pan, J.; Kugener, G.; Root, D. E.; Tsherniak, A. Extracting Biological Insights from the Project Achilles Genome-Scale CRISPR Screens in Cancer Cell Lines. bioRxiv 2019, 720243. https://doi.org/10.1101/720243, Meyers, R. M.; Bryan, J. G.; McFarland, J. M.; Weir, B. A.; Sizemore, A. E.; Xu, H.; Dharia, N. V.; Montgomery, P. G.; Cowley, G. S.; Pantel, S.; et al. Computational correction of copy number effect improves specificity of CRISPR-Cas9 essentiality screens in cancer cells. Nat. Genet. 2017, 49, 1779-1784. https://doi.org/10.1038/ng.3984] and Achilles combined with Sanger's Project SCORE (KY Cas9 library) [Behan, F. M.; Iorio, F.; Picco, G.; Goncalves, E.; Beaver, C. M.; Migliardi, G.; Santos, R.; Rao, Y.; Sassi, F.; Pinnelli, M.; et al. Prioritization of cancer therapeutic targets using CRISPR-Cas9 screens. Nature 2019, 568, 511-516. https://doi.org/i0.1038/s41586-019-1103-9] screens. The 22Q4 data release of the DepMap portal (accessed on 2 Mar. 2023 at https://depmap.org/portal/download/all/) provided the gene effect estimates for 48 breast cancer cell lines. Additionally, project Achilles (https://depmap.org/R2-D2/, accessed on 13 Apr. 2023) provided whole-genome RNAi screening data [McFarland, J. M.; Ho, Z. V.; Kugener, G.; Dempster, J. M.; Montgomery, P. G.; Bryan, J. G.; Krill-Burger, J. M.; Green, T. M.; Vazquez, F.; Boehm, J. S.; et al. Improved estimation of cancer dependencies from large-scale RNAi screens using model-based normalization and data integration. Nat. Commun. 2018, 9, 4610. https://doi.org/10.1038/s41467-018-06916-5] for CCLE cell lines. For our research, we used the genome-wide dependency scores of 34 breast cancer cell lines from RNAi screening.

Based on their significance to cell proliferation in each cell line, genes were categorized as essential and non-essential genes. In each cell line, the median of normalized dependence scores for common essential genes was −1, as compared to 0 for non-essential genes. We defined a significant effect of CRISPR-Cas9 knockout or RNAi knockdown as a normalized dependence score of less than −0.5 in this study.

Cancer Cell Line Encyclopedia (CCLE). We extracted comprehensive genetic information for accessible human breast cancer epithelial cell lines from the Cancer Cell Line Encyclopedia (CCLE) [Ghandi, M.; Huang, F. W.; Jane-Valbuena, J.; Kryukov, G. V.; Lo, C. C.; McDonald, E. R., 3rd; Barretina, J.; Gelfand, E. T.; Bielski, C. M.; Li, H.; et al. Next-generation characterization of the Cancer Cell Line Encyclopedia. Nature 2019, 569, 503-508. https://doi.org/10.1038/s41586-019-1186-3] release DepMap Public 22Q4 (https://depmap.org/portal/download/all/, accessed on 2 Mar. 2023). Specifically, we obtained RNA sequencing data for 63 breast cancer cell lines, 41,707 annotated and filtered mutations created with Mutect2, and 2979 fusions produced from RNAseq data.

Additionally, from a project by the Gygi lab [Nusinow, D. P.; Szpyt, J.; Ghandi, M.; Rose, C. M.; McDonald, E. R., 3rd; Kalocsay, M.; Jané-Valbuena, J.; Gelfand, E.; Schweppe, D. K.; Jedrychowski, M.; et al. Quantitative Proteomics of the Cancer Cell Line Encyclopedia. Cell 2020, 180, 387-402.e316. https://doi.org/10.1016/j.cell.2019.12.02](https://gygi.hms.harvard.edu/publications/ccle.html, accessed on 6 Mar. 2023), we acquired proteome information for 31 breast cancer cell lines. Both the mRNA and proteomic data were log 2-transformed, and the mean of protein expression was centered at 0.

Drug Sensitivity in CCLE. This study used drug sensitivity data of human breast cancer cell lines from various sources. The DepMap portal provided the secondary Profiling Relative Inhibition Simultaneously in Mixtures (PRISM) [Corsello, S. M.; Nagari, R. T.; Spangler, R. D.; Rossen, J.; Kocak, M.; Bryan, J. G.; Humeidi, R.; Peck, D.; Wu, X.; Tang, A. A.; et al. Discovering the anti-cancer potential of non-oncology drugs by systematic viability profiling. Nat. Cancer 2020, 1, 235-248. https://doi.org/10.1038/s43018-019-0018-6] repurposing dataset (PRISM repurposing 19Q4, accessed on 3 Apr. 2023), which includes screening results of 1447 compounds in 22 human breast cancer cell lines. Additionally, we downloaded the Genomics of Drug Sensitivity in Cancer (GDSC1 and GDSC2) datasets [Yang, W.; Soares, J.; Greninger, P.; Edelman, E. J.; Lightfoot, H.; Forbes, S.; Bindal, N.; Beare, D.; Smith, J. A.; Thompson, I. R.; et al. Genomics of Drug Sensitivity in Cancer (GDSC): A resource for therapeutic biomarker discovery in cancer cells. Nucleic Acids Res. 2013, 41, D955-D961. https://doi.org/10.1093/nar/gks1111, Iorio, F.; Knijnenburg, T. A.; Vis, D. J.; Bignell, G. R.; Menden, M. P.; Schubert, M.; Aben, N.; Goncalves, E.; Barthorpe, S.; Lightfoot, H.; et al. A Landscape of Pharmacogenomic Interactions in Cancer. Cell 2016, 166, 740-754. https://doi.org/10.1016/j.cell.2016.06.017, Garnett, M. J.; Edelman, E. J.; Heidorn, S. J.; Greenman, C. D.; Dastur, A.; Lau, K. W.; Greninger, P.; Thompson, I. R.; Luo, X.; Soares, J.; et al. Systematic identification of genomic markers of drug sensitivity in cancer cells. Nature 2012, 483, 570-575. https://doi.org/10.1038/nature11005] from the CancerRxGene website (https://www.cancerrxgene.org/downloads/bulk_download, accessed on 3 Apr. 2023). GDSC1 contains screening results of 51 breast cancer cell lines and 345 compounds, while GDSC2 includes 50 breast cancer cell lines and 190 compounds screened.

The drug activity measurements in these datasets, including IC50, ln(IC50), EC50, and ln(EC50), were used to investigate drug sensitivity in this study. Based on measures of their drug activity, the breast cancer cell lines from the CCLE dataset were divided into three groups: sensitive, resistant, and intermediate, which have been described in our prior works [Ye, Q.; Mohamed, R.; Dukhlallah, D.; Gencheva, M.; Hu, G.; Pearce, M. C.; Kolluri, S. K.; Marsh, C. B.; Eubank, T. D.; Ivanov, A. V.; et al. Molecular Analysis of ZNF71 KRAB in Non-Small-Cell Lung Cancer. Int. J. Mol. Sci. 2021, 22, 3752. https://doi.org/10.3390/ijms22073752, Ye, Q.; Singh, S.; Qian, P. R.; Guo, N. L. Immune-Omics Networks of CD27, PD1, and PDL1 in Non-Small Cell Lung Cancer. Cancers 2021, 13, 4296. https://doi.org/10.3390/cancers13174296].

CMap. Connectivity Map (CMap) is a bioinformatics software package that uses gene expression data to identify potential drugs or small molecules that can modulate specific biological pathways or disease states. By comparing the gene expression signature of a particular disease or cellular state with the gene expression profiles in CMap, drugs or small molecules that could potentially reverse or mimic the disease or cellular state can be identified. In this study, the CMap online tool (https://clue.io/, accessed on 7 Mar. 2023) was utilized to explore potential drug repositioning based on selected gene expression signatures. The results with a raw connectivity score>0.9 and a p-value<0.05 were considered significant and can be further investigated to provide insights into drug mechanism of action and identify potential therapeutic options.

Statistics Methods. This study utilized RStudio (version 2023.03.0 Build 386) with R version 4.2.1 as the primary tool for statistical analysis. The significance of differential expression between the two groups was evaluated using a two-tailed, unpaired Student's t-test. We employed the Kaplan-Meier method to generate survival curves and perform survival analysis and conducted log-rank tests to assess the difference in survival probability between different groups. The prognostic evaluation and risk score model building were performed with univariate and multivariate Cox regression analyses. The R packages “survival (version 3.5.3)” and “survminer (version 0.4.9)” were used. To determine the degree of a linear relationship between the two sample groups, we used Pearson's correlation test. A result was considered significant if the p-value was less than 0.05.

Conclusions. This study showed that the 28-gene signature previously reported to be prognostic of breast cancer and ovarian cancer clinical outcome is also diagnostic of breast cancer and can classify invasive cancer tumors from normal and ADH tissues. This gene assay was evaluated in multiple independent patient cohorts in solid tissues. This gene signature can further stratify basal-like and luminal A BRCA patients into different prognostic groups with distinct immune cell activities. Using seven genes within this assay, breast cancer patients could be separated from normal and DCIS patients using peripheral whole-blood samples. These results show its feasibility to be used in diagnostic tests on biopsy or as a minimally invasive test on patient blood samples, upon further validation with larger patient cohorts and prospective evaluation in the future. The functional involvement of multiple signature genes in breast cancer tumorigenesis was also confirmed in protein assays. PBX2 and RAD52 protein expression levels in IHC are prognostic in invasive breast cancer patients. The mRNA and protein expression of multiple signature genes is associated with response to 18 NCCN-recommended drugs for treating breast cancer. Eleven signature genes had a significant effect on human BRCA cell lines in CRISPR-Cas9/RNAi screening. Based on this 28-gene expression signature, the VEGFR inhibitor ZM-306416 was discovered with new indications for treating breast cancer. The results presented in this study indicated that the 28-gene signature can be used for improving diagnosis, treatment selection, and drug discovery for breast cancer.

The invention claimed is:

1. A method of diagnosing non-small cell lung cancer comprising
isolating RNA from tissue samples after surgical resection;
processing said RNA; and
identifying expression of a set of 73 microRNAs (miRNAs)
wherein the step of identification of said set of 73 miRNAs indicates existence or non-existence of non-small cell lung cancer with at least 91.7% accuracy.

2. The method of claim 1, wherein the set of 73 microRNAs comprise: hsa-miR-494, hsa-miR-933, hsa-miR-21*, hsa-miR-130b, hsa-miR-708, hsa-miR-193b, hsa-miR-1260, hsa-miR-31, hsa-miR-96, hsa-miR-183, hsa-miR-182, hsa-miR-7, hsa-miR-1274a, hsa-miR-411, hsa-miR-34b*, hsa-miR-34c-5p, hsa-miR-486-5p, hsa-miR-144, hsa-miR-363, hsa-miR-3184, hsa-miR-30e*, hsa-miR-338-3p, hsa-miR-138, hsa-miR-145*, hsa-miR-218, hsa-miR-139-5p, hsa-miR-30a*, hsa-miR-101, hsa-miR-133a, hsa-miR-133b, hsa-miR-126*, hsa-miR-4328, hsa-miR-338-5p, hsa-miR-30c-2*, hsa-miR-1, hsa-miR-3065-5p, hsa-miR-23a*, hsa-miR-27a*, hsa-miR-60b, hsa-miR-34b, hsa-miR-34c-3p, hsa-miR-223*, hsa-miR-218-1*, hsa-miR-144*, hsa-miR-3065-3p, hsa-miR-105, hsa-miR-183*, hsa-miR-31*, hsa-miR-301b, hsa-miR-196a, hsa-miR-9*, hsa-miR-9, hsa-miR-30c, hsa-miR-497, hsa-miR-99a, hsa-miR-335, hsa-miR-195, hsa-miR-30a, hsa-miR-223, hsa-miR-1280, hsa-miR-720, hsa-miR-141, hsa-miR-210, hsa-miR-205, hsa-miR-145, hsa-miR-143, hsa-miR-126, hsa-miR-30d, hsa-miR-30b, hsa-miR-451, hsa-miR-1260b, hsa-miR-4284, and hsa-miR-21.

3. The method of claim 1, further comprising treatment of non-small cell lung cancer in a patient in need thereof comprising administering to said patient a therapeutically effective amount of a compound selected from the group consisting of BX-912, daunorubicin, and midostaurin.

4. A method of distinguishing blood samples originating from lung cancer or healthy patients comprising,
processing RNA from said sample; and,
identifying expression of seven miRNAs that distinguishes blood samples from lung cancer patients and those from healthy individuals with an accuracy of 100%.

5. The method of claim 4, wherein the seven miRNAs comprise hsa-miR-449a, hsa-miR-200b, hsa-miR-20a, hsa-miR-191, hsa-miR-543, hsa-miR-26a and hsa-miR-1973.

6. A compound selected from the group consisting of BX-912, daunorubicin, and midostaurin for use as a repositioning drug for treating non-small cell lung cancer.

7. A method of treating non-small cell lung cancer in a patient in need thereof, comprising administering to said patient a therapeutically effective amount of a compound selected from the group consisting of BX-912, daunorubicin, and midostaurin.

8. A method of diagnosing breast cancer comprising
isolating RNA from tissue samples after surgical resection;
analyzing said RNA; and
identifying expression of a set of 86 microRNAs (miRNAs)
wherein the step of identification of said set of 86 miRNAs indicates existence or non-existence of breast cancer with at least 90.4% accuracy

9. The method of claim 8, wherein the set of 86 miRNAs comprise hsa-miR-1226, hsa-miR-4252, hsa-miR-449b*, hsa-miR-301b, hsa-miR-183*, hsa-miR-3119, hsa-miR-449a, hsa-miR-4328, hsa-miR-126*, hsa-miR-422a, hsa-miR-139-3p, hsa-miR-617, hsa-miR-489, hsa-miR-1229, hsa-miR-4279, hsa-miR-634, hsa-miR-615-3p, hsa-miR-33a, hsa-miR-7, hsa-miR-1274a, hsa-miR-1228, hsa-miR-1972, hsa-miR-218, hsa-miR-101, hsa-miR-204, hsa-miR-10b*, hsa-miR-486-5p, hsa-miR-670, hsa-miR-4316, hsa-miR-376a, hsa-miR-377, hsa-miR-154, hsa-miR-379, hsa-miR-139-5p, hsa-miR-145*, hsa-miR-30a*, hsa-miR-425, hsa-miR-1260, hsa-miR-193a-3p, hsa-miR-193a-5p, hsa-miR-155, hsa-miR-335, hsa-miR-4324, hsa-miR-608, hsa-miR-378, hsa-miR-378c, hsa-miR-376c, hsa-miR-96, hsa-miR-183, hsa-miR-182, hsa-miR-3124, hsa-miR-4258, hsa-miR-766, hsa-miR-4323, hsa-miR-1976, hsa-miR-1181, hsa-miR-1290, hsa-miR-130b, hsa-miR-21*, hsa-miR-933, hsa-miR-301a, hsa-miR-99a, hsa-miR-195, hsa-miR-30a, hsa-miR-451, hsa-miR-199b-5p, hsa-miR-100, hsa-miR-10b, hsa-miR-497, hsa-miR-1280, hsa-miR-4286, hsa-miR-1469, hsa-miR-720, hsa-miR-1246, hsa-miR-145, hsa-miR-143, hsa-miR-126, hsa-miR-205, hsa-miR-1260b, hsa-miR-21, and hsa-miR-3172.

10. A method of distinguishing blood samples originating from breast cancer comprising,
processing RNA from said sample; and,
identifying expression of six miRNAs that distinguishes blood samples from breast cancer patients and those from healthy individuals with an accuracy of up to 100%.

11. The method of claim 10, wherein the six miRNAs comprise hsa-miR-30a*, hsa-miR-224*, hsa-miR-154, hsa-miR-155, hsa-miR-1972, and hsa-miR-3172.

12. A compound selected from MEK inhibitors PD19830, BRD-K12242279, pilocarpine, and tremorine for use as a repositioning drug for treating breast cancer.

13. A method of treating breast cancer in a patient in need thereof comprising administering to said patient a therapeutically effective amount of a compound selected from the group consisting of MEK inhibitors PD19830, BRD-K12244279, pilocarpine and tremorine.

14. A method of identifying invasive ductal carcinomas in blood samples, comprising
conducting real time PCR in said samples;
quantifying mRNA expression of 28 genes of ACOT4, DDOST, FAT, FGF2, HSP90AB1, IGHA1, INPPL1, INSMI, IRF5, MAP2K2, MCF2, MCM2, PBX2, PDGFRA, PSMC3IP, PLSCR1, RAD50, RAD52, RPUSD2, S100P, SEH1L, SLC25A5, SMARCD2, SSBP1, TOMM70A, and TXNRD1;
normalizing the quantification of said 28 genes with the quantification of control genes; and,
utilizing said quantification to determine whether said blood sample is positive for invasive ductal carcinoma.

15. A compound VEGFR inhibitor ZM-306416 for use as a new drug for treating breast cancer.

16. A method of treating breast cancer in a patient in need thereof comprising administering to said patient a therapeutically effective amount of VEGFR inhibitor ZM-306416.
